(19) United States
(12) Patent Application Publication
(10) Pub. No.: US 2024/0350145 A1
(43) Pub. Date: Oct. 24, 2024

(54) Intrasaccular Aneurysm Occlusion Device with a Convex Distal Portion Nested within a Concave Proximal Portion

(71) Applicant: Robert A. Connor, Wyoming, MN (US)

(72) Inventor: Robert A. Connor, Wyoming, MN (US)

(73) Assignee: Aneuclose LLC, Ham Lake, MN (US)

(21) Appl. No.: 18/760,322

(22) Filed: Jul. 1, 2024

Related U.S. Application Data
(63) Continuation-in-part of application No. 18/674,996, filed on May 27, 2024.
(63) Continuation-in-part of application No. 18/613,053, filed on Mar. 21, 2024.
(63) Continuation-in-part of application No. 17/970,510, filed on Oct. 20, 2022.
Application No. 18/674,996 is a continuation-in-part of application No. 18/613,053, filed on Mar. 21, 2024.
Application No. 18/613,053 is a continuation-in-part of application No. 18/519,055, filed on Nov. 26, 2023, now Pat. No. 12,004,750.
Application No. 18/613,053 is a continuation-in-part of application No. 18/519,055, filed on Nov. 26, 2023, now Pat. No. 12,004,750.
Application No. 18/519,055 is a continuation-in-part of application No. 18/135,153, filed on Apr. 15, 2023, now abandoned.
Application No. 18/519,055 is a continuation-in-part of application No. 18/374,602, filed on Sep. 28, 2023, now Pat. No. 11,944,316.
Application No. 18/374,602 is a continuation-in-part of application No. 18/135,153, filed on Apr. 15, 2023, now abandoned.
Application No. 18/135,153 is a continuation-in-part of application No. 17/970,510, filed on Oct. 20, 2022.
Application No. 17/970,510 is a continuation-in-part of application No. 17/965,502, filed on Oct. 13, 2022, now abandoned.
Application No. 17/965,502 is a continuation-in-part of application No. 17/829,313, filed on May 31, 2022, now abandoned.
Application No. 18/374,602 is a continuation-in-part of application No. 18/135,153, filed on Apr. 15, 2023, now abandoned.
Application No. 18/135,153 is a continuation-in-part of application No. 17/970,510, filed on Oct. 20, 2022.
Application No. 17/970,510 is a continuation-in-part of application No. 17/965,502, filed on Oct. 13, 2022, now abandoned.
Application No. 17/965,502 is a continuation-in-part of application No. 17/829,313, filed on May 31, 2022, now abandoned.
Application No. 18/135,153 is a continuation-in-part of application No. 17/970,510, filed on Oct. 20, 2022.
Application No. 17/970,510 is a continuation-in-part of application No. 17/965,502, filed on Oct. 13, 2022, now abandoned.
Application No. 17/965,502 is a continuation-in-part of application No. 17/829,313, filed on May 31, 2022, now abandoned.
Application No. 17/970,510 is a continuation-in-part of application No. 17/965,502, filed on Oct. 13, 2022, now abandoned.
Application No. 17/965,502 is a continuation-in-part of application No. 17/829,313, filed on May 31, 2022, now abandoned.
Application No. 17/829,313 is a continuation-in-part of application No. 17/476,845, filed on Sep. 16, 2021, now Pat. No. 11,583,289.
Application No. 17/965,502 is a continuation-in-part of application No. 17/829,313, filed on May 31, 2022, now abandoned.
Application No. 17/829,313 is a continuation-in-part of application No. 17/476,845, filed on Sep. 16, 2021, now Pat. No. 11,583,289.
Application No. 17/829,313 is a continuation-in-part of application No. 17/485,390, filed on Sep. 25, 2021, now Pat. No. 11,471,164.
Application No. 17/485,390 is a continuation-in-part of application No. 17/476,845, filed on Sep. 16, 2021, now Pat. No. 11,583,289.
Application No. 17/476,845 is a continuation-in-part of application No. 17/472,674, filed on Sep. 12, 2021, now abandoned.
Application No. 17/472,674 is a continuation-in-part of application No. 17/467,680, filed on Sep. 7, 2021, now abandoned.
Application No. 17/467,680 is a continuation-in-part of application No. 17/466,497, filed on Sep. 3, 2021, now Pat. No. 11,357,511.
Application No. 17/466,497 is a continuation-in-part of application No. 17/353,652, filed on Jun. 21, 2021, now abandoned.
Application No. 17/353,652 is a continuation-in-part of application No. 17/220,002, filed on Apr. 1, 2021, now Pat. No. 11,464,518.
Application No. 17/220,002 is a continuation-in-part of application No. 17/214,827, filed on Mar. 27, 2021, now Pat. No. 11,484,322.
Application No. 17/214,827 is a continuation-in-part of application No. 17/211,446, filed on Mar. 24, 2021, now abandoned.
Application No. 17/220,002 is a continuation-in-part of application No. 17/214,827, filed on Mar. 27, 2021, now Pat. No. 11,484,322.
Application No. 17/214,827 is a continuation-in-part of application No. 17/211,446, filed on Mar. 24, 2021, now abandoned.
Application No. 17/211,446 is a continuation-in-part of application No. 16/693,267, filed on Nov. 23, 2019, now abandoned.
Application No. 16/693,267 is a continuation-in-part of application No. 16/660,929, filed on Oct. 23, 2019, now Pat. No. 11,471,163.
Application No. 16/693,267 is a continuation-in-part of application No. 16/660,929, filed on Oct. 23, 2019, now Pat. No. 11,471,163.
Application No. 16/660,929 is a continuation-in-part of application No. 16/541,241, filed on Aug. 15, 2019, now abandoned.
Application No. 16/541,241 is a continuation-in-part of application No. 15/865,822, filed on Jan. 9, 2018, now Pat. No. 10,716,573.
Application No. 15/865,822 is a continuation-in-part of application No. 15/861,482, filed on Jan. 3, 2018, now abandoned.
Application No. 15/861,482 is a continuation-in-part of application No. 16/541,241, filed on Aug. 15, 2019, now abandoned.
Application No. 16/541,241 is a continuation-in-part of application No. 15/865,822, filed on Jan. 9, 2018, now Pat. No. 10,716,573.
Application No. 15/865,822 is a continuation-in-part of application No. 15/861,482, filed on Jan. 3, 2018, now abandoned.
Application No. 15/861,482 is a continuation-in-part of application No. 15/865,822, filed on Jan. 9, 2018, now Pat. No. 10,716,573.
Application No. 15/865,822 is a continuation-in-part of application No. 15/081,909, filed on Mar. 27, 2016, now abandoned.
Application No. 15/081,909 is a continuation-in-part of application No. 14/526,600, filed on Oct. 29, 2014, now abandoned.
Application No. 14/526,600 is a continuation-in-part of application No. 15/080,915, filed on Mar. 25, 2016, now Pat. No. 10,028,747.
Application No. 15/080,915 is a continuation-in-part of application No. 14/526,600, filed on Oct. 29, 2014, now abandoned.
Application No. 15/081,909 is a continuation-in-part of application No. 14/526,600, filed on Oct. 29, 2014, now abandoned.
Application No. 15/080,915 is a continuation-in-part of application No. 14/526,600, filed on Oct. 29, 2014, now abandoned.
Application No. 14/526,600 is a continuation-in-part of application No. 12/989,048, filed on Oct. 21, 2010, now Pat. No. 8,974,487.
(60) Provisional application No. 63/119,774, filed on Dec. 1, 2020.
(60) Provisional application No. 62/794,609, filed on Jan. 19, 2019.
(60) Provisional application No. 62/794,607, filed on Jan. 19, 2019.
(60) Provisional application No. 62/794,609, filed on Jan. 19, 2019.
(60) Provisional application No. 62/794,607, filed on Jan. 19, 2019.
(60) Provisional application No. 62/794,609, filed on Jan. 19, 2019.
(60) Provisional application No. 62/794,607, filed on Jan. 19, 2019.
(60) Provisional application No. 62/720,173, filed on Aug. 21, 2018.
(60) Provisional application No. 62/589,754, filed on Nov. 22, 2017.
(60) Provisional application No. 62/472,519, filed on Mar. 16, 2017.
(60) Provisional application No. 62/589,754, filed on Nov. 22, 2017.
(60) Provisional application No. 62/472,519, filed on Mar. 16, 2017.
(60) Provisional application No. 62/444,860, filed on Jan. 11, 2017.
(60) Provisional application No. 61/897,245, filed on Oct. 30, 2013.
(60) Provisional application No. 61/126,047, filed on May 1, 2008.
(60) Provisional application No. 61/126,027, filed on May 1, 2008.

(51) Int. Cl. A61B 17/12

(52) U.S. Cl. CPC A61B 17/12172; A61B 17/12113; A61B 17/12177

(57) ABSTRACT

Disclosed herein in an intrasaccular aneurysm occlusion device with a convex (e.g. globular or heart-shaped) distal portion which is at least partially nested in a concave (e.g. bowl-shaped) proximal portion. In an example, the convex distal portion and the concave proximal portion can be made by radially-constraining, inverting, and/or everting a tubular mesh. In another example, a convex distal portion and a concave proximal portion can be made separately from different materials and/or different processes. The convex distal portion can be filled with embolic coils, “string-of-pearls” embolic strands, or congealing liquid.

### CROSS-REFERENCE TO RELATED APPLICATIONS

This application is a continuation-in-part of U.S. patent application Ser. No. 18/674,996 filed on 2024 May 27, a continuation-in-part of U.S. patent application Ser. No. 18/613,053 filed on 2024 Mar. 21, and a continuation-in-part of U.S. patent application Ser. No. 17/970,510 filed on 2022 Oct. 20.

U.S. patent application Ser. No. 18/674,996 was a continuation-in-part of Ser. No. 18/613,053 filed on 2024 Mar. 21 and a continuation-in-part of U.S. patent application Ser. No. 18/519,055 filed on 2023 Nov. 26.

U.S. patent application Ser. No. 18/613,053 was a continuation-in-part of U.S. patent application Ser. No. 18/519,055 filed on 2023 Nov. 26 and a continuation-in-part of U.S. patent application Ser. No. 18/135,153 filed on 2023 Apr. 15.

U.S. patent application Ser. No. 18/519,055 was a continuation-in-part of U.S. patent application Ser. No. 18/374,602 filed on 2023 Sep. 28, a continuation-in-part of U.S. patent application Ser. No. 18/135,153 filed on 2023 Apr. 15, a continuation-in-part of U.S. patent application Ser. No. 17/970,510 filed on 2022 Oct. 20, a continuation-in-part of U.S. patent application Ser. No. 17/965,502 filed on 2022 Oct. 13, and a continuation-in-part of U.S. patent application Ser. No. 17/829,313 filed on 2022 May 31.

U.S. patent application Ser. No. 18/374,602 was a continuation-in-part of U.S. patent application Ser. No. 18/135,153 filed on 2023 Apr. 15, a continuation-in-part of U.S. patent application Ser. No. 17/970,510 filed on 2022 Oct. 20, a continuation-in-part of U.S. patent application Ser. No. 17/965,502 filed on 2022 Oct. 13, and a continuation-in-part of U.S. patent application Ser. No. 17/829,313 filed on 2022 May 31.

U.S. patent application Ser. No. 18/135,153 was a continuation-in-part of U.S. patent application Ser. No. 17/970,510 filed on 2022 Oct. 20, a continuation-in-part of U.S. patent application Ser. No. 17/965,502 filed on 2022 Oct. 13, and a continuation-in-part of U.S. patent application Ser. No. 17/829,313 filed on 2022 May 31. U.S. patent application Ser. No. 17/970,510 was a continuation-in-part of U.S. patent application Ser. No. 17/965,502 filed on 2022 Oct. 13, a continuation-in-part of U.S. patent application Ser. No. 17/829,313 filed on 2022 May 31, and a continuation-in-part of U.S. patent application Ser. No. 17/476,845 filed on 2021 Sep. 16.

U.S. patent application Ser. No. 17/829,313 was a continuation-in-part of U.S. patent application Ser. No. 17/485,390 filed on 2021 Sep. 25, was a continuation-in-part of U.S. patent application Ser. No. 17/476,845 filed on 2021 Sep. 16, was a continuation-in-part of U.S. patent application Ser. No. 17/472,674 filed on 2021 Sep. 12, was a continuation-in-part of U.S. patent application Ser. No. 17/467,680 filed on 2021 Sep. 7, was a continuation-in-part of U.S. patent application Ser. No. 17/466,497 filed on 2021 Sep. 3, was a continuation-in-part of U.S. patent application Ser. No. 17/353,652 filed on 2021 Jun. 21, was a continuation-in-part of U.S. patent application Ser. No. 17/220,002 filed on 2021 Apr. 1, was a continuation-in-part of U.S. patent application Ser. No. 17/214,827 filed on 2021 Mar. 27, was a continuation-in-part of U.S. patent application Ser. No. 17/211,446 filed on 2021 Mar. 24, was a continuation-in-part of U.S. patent application Ser. No. 16/693,267 filed on 2019 Nov. 23, and was a continuation-in-part of U.S. patent application Ser. No. 16/660,929 filed on 2019 Oct. 23.

U.S. patent application Ser. No. 17/220,002 was a continuation-in-part of U.S. patent application Ser. No. 17/214,827 filed on 2021 Mar. 27. U.S. patent application Ser. No. 17/220,002 was a continuation-in-part of U.S. patent application Ser. No. 17/211,446 filed on 2021 Mar. 24. U.S. patent application Ser. No. 17/220,002 claimed the priority benefit of U.S. provisional patent application 63/119,774 filed on 2020 Dec. 1. U.S. patent application Ser. No. 17/220,002 was a continuation-in-part of U.S. patent application Ser. No. 16/693,267 filed on 2019 Nov. 23. U.S. patent application Ser. No. 17/220,002 was a continuation-in-part of U.S. patent application Ser. No. 16/660,929 filed on 2019 Oct. 23.

U.S. patent application Ser. No. 16/693,267 was a continuation-in-part of U.S. patent application Ser. No. 16/660,929 filed on 2019 Oct. 23. U.S. patent application Ser. No. 16/693,267 claimed the priority benefit of U.S. provisional patent application 62/794,609 filed on 2019 Jan. 19. U.S. patent application Ser. No. 16/693,267 claimed the priority benefit of U.S. provisional patent application 62/794,607 filed on 2019 Jan. 19. U.S. patent application Ser. No. 16/693,267 was a continuation-in-part of U.S. patent application Ser. No. 16/541,241 filed on 2019 Aug. 15. U.S. patent application Ser. No. 16/693,267 was a continuation-in-part of U.S. patent application Ser. No. 15/865,822 filed on 2018 Jan. 9 which issued as U.S. Pat. No. 10,716,573 on 2020 Jul. 21. U.S. patent application Ser. No. 16/693,267 was a continuation-in-part of U.S. patent application Ser. No. 15/861,482 filed on 2018 Jan. 3.

U.S. patent application Ser. No. 16/660,929 claimed the priority benefit of U.S. provisional patent application 62/794,609 filed on 2019 Jan. 19. U.S. patent application Ser. No. 16/660,929 claimed the priority benefit of U.S. provisional patent application 62/794,607 filed on 2019 Jan. 19. U.S. patent application Ser. No. 16/660,929 was a continuation-in-part of U.S. patent application Ser. No. 16/541,241 filed on 2019 Aug. 15. U.S. patent application Ser. No. 16/660,929 was a continuation-in-part of U.S. patent application Ser. No. 15/865,822 filed on 2018 Jan. 9 which issued as U.S. Pat. No. 10,716,573 on 2020 Jul. 21. U.S. patent application Ser. No. 16/660,929 was a continuation-in-part of U.S. patent application Ser. No. 15/861,482 filed on 2018 Jan. 3.

U.S. patent application Ser. No. 16/541,241 claimed the priority benefit of U.S. provisional patent application 62/794,609 filed on 2019 Jan. 19. U.S. patent application Ser. No. 16/541,241 claimed the priority benefit of U.S. provisional patent application 62/794,607 filed on 2019 Jan. 19. U.S. patent application Ser. No. 16/541,241 claimed the priority benefit of U.S. provisional patent application 62/720,173 filed on 2018 Aug. 21. U.S. patent application Ser. No. 16/541,241 was a continuation-in-part of U.S. patent application Ser. No. 15/865,822 filed on 2018 Jan. 9 which issued as U.S. Pat. No. 10,716,573 on 2020 Jul. 21

U.S. patent application Ser. No. 15/865,822 claimed the priority benefit of U.S. provisional patent application 62/589,754 filed on 2017 Nov. 22. U.S. patent application Ser. No. 15/865,822 claimed the priority benefit of U.S. provisional patent application 62/472,519 filed on 2017 Mar. 16. U.S. patent application Ser. No. 15/865,822 was a continuation-in-part of U.S. patent application Ser. No. 15/081,909 filed on 2016 Mar. 27. U.S. patent application Ser. No. 15/865,822 was a continuation-in-part of U.S. patent application Ser. No. 14/526,600 filed on 2014 Oct. 29.

U.S. patent application Ser. No. 15/861,482 claimed the priority benefit of U.S. provisional patent application 62/589,754 filed on 2017 Nov. 22. U.S. patent application Ser. No. 15/861,482 claimed the priority benefit of U.S. provisional patent application 62/472,519 filed on 2017 Mar. 16. U.S. patent application Ser. No. 15/861,482 claimed the priority benefit of U.S. provisional patent application 62/444,860 filed on 2017 Jan. 11. U.S. patent application Ser. No. 15/861,482 was a continuation-in-part of U.S. patent application Ser. No. 15/080,915 filed on 2016 Mar. 25 which issued as U.S. Pat. No. 10,028,747 on 2018 Jul. 24. U.S. patent application Ser. No. 15/861,482 was a continuation-in-part of U.S. patent application Ser. No. 14/526,600 filed on 2014 Oct. 29.

U.S. patent application Ser. No. 15/081,909 was a continuation-in-part of U.S. patent application Ser. No. 14/526,600 filed on 2014 Oct. 29. U.S. patent application Ser. No. 15/080,915 was a continuation-in-part of U.S. patent application Ser. No. 14/526,600 filed on 2014 Oct. 29. U.S. patent application Ser. No. 14/526,600 claimed the priority benefit of U.S. provisional patent application 61/897,245 filed on 2013 Oct. 30. U.S. patent application Ser. No. 14/526,600 was a continuation-in-part of U.S. patent application Ser. No. 12/989,048 filed on 2010 Oct. 21 which issued as U.S. Pat. No. 8,974,487 on 2015 Mar. 10. U.S. patent application Ser. No. 12/989,048 claimed the priority benefit of U.S. provisional patent application 61/126,047 filed on 2008 May 1. U.S. patent application Ser. No. 12/989,048 claimed the priority benefit of U.S. provisional patent application 61/126,027 filed on 2008 May 1.

The entire contents of these related applications are incorporated herein by reference.

### FEDERALLY SPONSORED RESEARCH

Not Applicable

### SEQUENCE LISTING OR PROGRAM

Not Applicable

### BACKGROUND

### Field of Invention

This invention relates to aneurysm occlusion devices and methods.

### Introduction

An aneurysm is an abnormal bulging of a blood vessel wall. The vessel from which the aneurysm protrudes is the parent vessel. Saccular aneurysms look like a sac protruding out from the parent vessel. Saccular aneurysms have a neck and can be prone to rupture. Fusiform aneurysms are a form of aneurysm in which a blood vessel is expanded circumferentially in all directions. Fusiform aneurysms generally do not have a neck and are less prone to rupturing than saccular aneurysms. As an aneurysm grows larger, its walls generally become thinner and weaker. This decrease in wall integrity, particularly for saccular aneurysms, increases the risk of the aneurysm rupturing and hemorrhaging blood into the surrounding tissue, with serious and potentially fatal health outcomes.

Cerebral aneurysms, also called brain aneurysms or intracranial aneurysms, are aneurysms that occur in the intercerebral arteries that supply blood to the brain. The majority of cerebral aneurysms form at the junction of arteries at the base of the brain that is known as the Circle of Willis where arteries come together and from which these arteries send branches to different areas of the brain. Although identification of intact aneurysms is increasing due to increased use of outpatient imaging such as outpatient MRI scanning, many cerebral aneurysms still remain undetected unless they rupture. If they do rupture, they often cause stroke, disability, and/or death. The prevalence of cerebral aneurysms is generally estimated to be in the range of 1%-5% of the general population or approximately 3-15 million people in the U.S. alone. Approximately 30,000 people per year suffer a ruptured cerebral aneurysm in the U.S. alone. Approximately one-third to one-half of people who suffer a ruptured cerebral aneurysm die within one month of the rupture. Even among those who survive, approximately one-half suffer significant and permanent deterioration of brain function. Better alternatives for cerebral aneurysm treatment are needed.

### REVIEW OF THE RELEVANT ART

U.S. patent application 20020169473 (Sepetka et al., Nov. 14, 2002, “Devices and Methods for Treating Vascular Malformations”) discloses a device with a primary coil to provide structural integrity and secondary windings to fill interstitial spaces. U.S. patent applications 20060155323 (Porter et al., Jul. 13, 2006, “Intra-Aneurysm Devices”) and 20190298379 (Porter et al., Oct. 3, 2019, “Intra-Aneurysm Devices”) and also U.S. Pat. No. 10,265,075 (Porter et al., Apr. 23, 2019, “Intra-Aneurysm Devices”) disclose an occlusive device having a neck and a dome. U.S. patent application 20080281350 (Sepetka et al., Nov. 13, 2008, “Aneurysm Occlusion Devices”) discloses an implantable occlusion device with a concave or cup-shaped shape after implantation.

U.S. patent application 20110022149 (Cox et al., Jan. 27, 2011, “Methods and Devices for Treatment of Vascular Defects”) discloses a device with first ends secured to a first ring and second ends secured to a second ring with the first and second rings being disposed substantially concentric to the longitudinal axis. U.S. patent application 20110208227 (Becking, Aug. 25, 2011, “Filamentary Devices for Treatment of Vascular Defects”) discloses braid-balls for aneurysm occlusion. U.S. patent application 20120165919 (Cox et al., Jun. 28, 2012, “Methods and Devices for Treatment of Vascular Defects”) and U.S. patent application 20140052233 (Cox et al., Feb. 20, 2014, “Methods and Devices for Treatment of Vascular Defects”) disclose an expandable wire body support structure having a low profile radially constrained state, an expanded relaxed state with a substantially spherical or globular configuration having a smooth outer surface, and a porous permeable layer comprising a braided wire occlusive mesh.

U.S. patent application 20120283768 (Cox et al., Nov. 8, 2012, “Method and Apparatus for the Treatment of Large and Giant Vascular Defects”) discloses the deployment of multiple permeable shell devices within a single vascular defect. U.S. patent application 20120316598 (Becking et al., Dec. 13, 2012, “Multiple Layer Filamentary Devices for Treatment of Vascular Defects”) and U.S. Pat. No. 9,585,669 (Becking et al., Mar. 17, 2017, “Multiple Layer Filamentary Devices for Treatment of Vascular Defects”) disclose braid balls for aneurysm occlusion. U.S. patent application 20130245667 (Marchand et al., Sep. 19, 2013, “Filamentary Devices and Treatment of Vascular Defects”) discloses a self-expanding resilient permeable shell wherein filaments are bundled and secured to each other at a proximal end.

U.S. Pat. No. 8,597,320 (Sepetka et al., Dec. 3, 2013, “Devices and Methods for Treating Vascular Malformations”) discloses a method of filling an aneurysm by advancing a device with a proximal collar and a distal collar through a vascular system and then positioning the device within an aneurysm. U.S. patent applications 20140135811 (Divino et al., May 15, 2014, “Occlusive Devices”), 20140135812 (Divino et al., May 15, 2014, “Occlusive Devices”), 20190282242 (Divino et al., Sep. 19, 2019, “Occlusive Devices”), and 20190290286 (Divino et al., Sep. 26, 2019, “Occlusive Devices”) and also U.S. Pat. No. 10,327,781 (Divino et al., Jun. 25, 2019, “Occlusive Devices”) disclose multiple expandable structures, wherein each of the expandable structures has a unique shape or porosity profile.

U.S. patent application 20140200607 (Sepetka et al., Jul. 17, 2014, “Occlusive Device”), U.S. patent application 20190274691 (Sepetka et al., Sep. 12, 2019, “Occlusive Device”), and U.S. Pat. No. 11,045,203 (Sepetka et al., Jun. 29, 2021, “Occlusive Device”) disclose multiple sequentially deployed occlusive devices that are connected together to create an extended length. U.S. patent application 20140330299 (Rosenbluth et al., Nov. 6, 2014, “Embolic Occlusion Device and Method”), U.S. patent application 20180303486 (Rosenbluth et al., Oct. 25, 2018, “Embolic Occlusion Device and Method”), and U.S. patent application 20210259699 (Rosenbluth et al., Aug. 26, 2021, “Embolic Occlusion Device and Method”) disclose an occlusion device with a tubular braided member having a first end and a second end and extending along a longitudinal axis, the tubular braided member having a repeating pattern of larger diameter portions and smaller diameter portions arrayed along the longitudinal axis.

U.S. patent application 20140358178 (Hewitt et al., Dec. 4, 2014, “Filamentary Devices for Treatment of Vascular Defects”), U.S. Pat. No. 9,078,658 (Hewitt et al., Jul. 14, 2015, “Filamentary Devices for Treatment of Vascular Defects”), U.S. patent application 20160249934 (Hewitt et al., Sep. 1, 2016, “Filamentary Devices for Treatment of Vascular Defects”), U.S. Pat. No. 9,955,976 (Hewitt et al., May 1, 2018, “Filamentary Devices for Treatment of Vascular Defects”), U.S. patent application 20180206849 (Hewitt et al., Jul. 26, 2018, “Filamentary Devices for the Treatment of Vascular Defects”). U.S. patent application 20210007754 (Milhous et al., Jan. 14, 2021, “Filamentary Devices for Treatment of Vascular Defects”), U.S. Pat. No. 10,939,914 (Hewitt et al., Mar. 9, 2021, “Filamentary Devices for the Treatment of Vascular Defects”), and U.S. patent application 20210275184 (Hewitt et al., Sep. 9, 2021, “Filamentary Devices for Treatment of Vascular Defects”) disclose occlusion devices with permeable shells made of woven braided mesh having a variable mesh density and/or porosity.

U.S. Pat. No. 8,998,947 (Aboytes et al., Apr. 7, 2015, “Devices and Methods for the Treatment of Vascular Defects”) discloses an expandable implant that with a first configuration in which the first portion and the second portion are substantially linearly aligned and a second configuration in which the second portion at least partially overlaps the first portion. U.S. Pat. No. 9,039,726 (Becking, May 26, 2015, “Filamentary Devices for Treatment of Vascular Defects”) discloses braid-balls for aneurysm occlusion. U.S. patent applications 20150272589 (Lorenzo, Oct. 1, 2015, “Aneurysm Occlusion Device”) and 20190008522 (Lorenzo, Jan. 10, 2019, “Aneurysm Occlusion Device”) and also U.S. Pat. No. 11,076,860 (Lorenzo, Aug. 3, 2021, “Aneurysm Occlusion Device”) disclose a tubular structure which is constrained by a control ring.

U.S. patent applications 20150313605 (Griffin, Nov. 5, 2015, “Occlusion Device”) and 20190059909 (Griffin, Feb. 28, 2019, “Occlusion Device”) and also U.S. Pat. No. 10,130,372 (Griffin, Nov. 20, 2018, “Occlusion Device”) and U.S. Pat. No. 11,389,174 (Griffin, Jul. 19, 2022, “Occlusion Device”) disclose an occlusion device with a substantially solid marker having a proximal end, and a distal end; and a low profile resilient mesh body attached to the distal end of the marker. U.S. patent applications 20160022445 (Ruvalcaba et al., Jan. 28, 2016, “Occlusive Device”) and 20190343664 (Ruvalcaba et al., Nov. 14, 2019, “Occlusive Device”) U.S. Pat. No. 11,389,309 (Ruvalcaba et al., Jul. 19, 2022, “Occlusive Device”) disclose an aneurysm embolization device having a single, continuous piece of material that is shape set into a plurality of distinct structural components.

U.S. patent application 20160249935 (Hewitt et al., Sep. 1, 2016, “Devices for Therapeutic Vascular Procedures”), U.S. patent application 20160367260 (Hewitt et al., Dec. 22, 2016, “Devices for Therapeutic Vascular Procedures”), U.S. Pat. No. 9,629,635 (Hewitt et al., Apr. 25, 2017, “Devices for Therapeutic Vascular Procedures”), and U.S. patent application 20170128077 (Hewitt et al., May 11, 2017, “Devices for Therapeutic Vascular Procedures”) disclose a self-expanding resilient permeable shell and a metallic coil secured to the distal end of the permeable shell. U.S. patent application 20160249937 (Marchand et al., Sep. 1, 2016, “Multiple Layer Filamentary Devices for Treatment of Vascular Defects”), U.S. Pat. No. 9,918,720 (Marchand et al., Mar. 20, 2018, “Multiple Layer Filamentary Devices for Treatment of Vascular Defects”), and U.S. Pat. No. 10,238,393 (Marchand et al., Mar. 26, 2019, “Multiple Layer Filamentary Devices for Treatment of Vascular Defects”) disclose a permeable shell and an inner structure configured to occlude blood flow.

U.S. Pat. No. 9,492,174 (Hewitt et al., Nov. 15, 2016, “Filamentary Devices for Treatment of Vascular Defects”), U.S. patent application 20170095254 (Hewitt et al., Apr. 6, 2017, “Filamentary Devices for Treatment of Vascular Defects”), U.S. Pat. No. 10,136,896 (Hewitt et al., Nov. 27, 2018, “Filamentary Devices for Treatment of Vascular Defects”), U.S. patent application 20190192166 (Hewitt et al., Jun. 27, 2019, “Filamentary Devices for Treatment of Vascular Defects”), U.S. patent application 20200289124 (Rangwala et al., Sep. 17, 2020, “Filamentary Devices for Treatment of Vascular Defects”), U.S. Pat. No. 10,813,645 (Hewitt et al., Oct. 27, 2020, “Filamentary Devices for Treatment of Vascular Defects”), and U.S. patent application 20210106337 (Hewitt et al., Apr. 15, 2021, “Filamentary Devices for Treatment of Vascular Defects”) disclose a self-expanding permeable shell having a radially constrained elongated state configured for delivery within a catheter lumen, an expanded state with a globular and longitudinally shortened configuration relative to the radially constrained state, and a plurality of elongate filaments that are woven together.

U.S. patent application 20170079661 (Bardsley et al., Mar. 23, 2017, “Occlusive Devices”), U.S. Pat. No. 10,314,593 (Bardsley et al., Jun. 11, 2019, “Occlusive Devices”), and U.S. patent application 20190269411 (Bardsley et al., Sep. 5, 2019, “Occlusive Devices”) disclose an implant with a single- or dual-layer braided body with variable porosity. U.S. patent application 20170079662 (Rhee et al., Mar. 23, 2017. “Occlusive Devices”) discloses an implant with a frame and a mesh component, wherein the mesh component has a first porosity and the frame has a second porosity. U.S. patent application 20170156734 (Griffin, Jun. 8, 2017, “Occlusion Device”), U.S. Pat. No. 10,285,711 (Griffin, May 14, 2019, “Occlusion Device”), U.S. patent application 20190269414 (Griffin, Sep. 5, 2019, “Occlusion Device”), U.S. patent application 20210153871 (Griffin, May 27, 2021, “Occlusion Device”), and U.S. patent application 20220313274 (Griffin, Oct. 6, 2022, “Occlusion Device”) disclose a continuous compressible mesh structure comprising axial mesh carriages configured end to end, wherein each end of each carriage is a pinch point in the continuous mesh structure.

U.S. patent application 20170224350 (Shimizu et al., Aug. 10, 2017, “Devices for Vascular Occlusion”). U.S. Pat. No. 10,729,447 (Shimizu et al., Aug. 4, 2020, “Devices for Vascular Occlusion”), U.S. patent application 20200323534 (Shimizu et al., Oct. 15, 2020, “Devices for Vascular Occlusion”). U.S. Pat. No. 10,980,545 (Bowman et al., Apr. 20, 2021, “Devices for Vascular Occlusion”), U.S. patent application 20210228214 (Bowman et al., Jul. 29, 2021, “Devices for Vascular Occlusion”), and U.S. patent application 20210228214 (Bowman et al., Jul. 29, 2021, “Devices for Vascular Occlusion”) disclose an occlusive device, an occlusive device delivery system, method of using, method of delivering an occlusive device, and method of making an occlusive device to treat various intravascular conditions. U.S. patent application 20170258473 (Plaza et al., Sep. 14, 2017, “Systems and Methods for Delivery of Stents and Stent-Like Devices”) discloses an elongate tubular member having a lumen, an expandable stent, and a delivery device which is placed in a cerebral vessel adjacent to an aneurysm.

U.S. patent application 20170281194 (Divino et al., Oct. 5, 2017, “Embolic Medical Devices”) discloses an occlusive device with an elongate member having opposing first and second side edges which extend longitudinally along the member and a member width, wherein this member has a collapsed configuration in which the first and second side edges are curled toward each other about a longitudinal axis of the member. U.S. patent application 20180000489 (Marchand et al., Jan. 4, 2018, “Filamentary Devices for Treatment of Vascular Defects”) discloses a self-expanding resilient permeable shell having a plurality of elongate resilient filaments with a woven structure. U.S. patent application 20180070955 (Greene et al., Mar. 15, 2018, “Embolic Containment”) discloses a method of treating a neurovascular arteriovenous malformation with liquid embolic and dimethyl sulfoxide.

U.S. patent application 20180242979 (Lorenzo, Aug. 30, 2018, “Aneurysm Device and Delivery System”) and U.S. Pat. No. 10,751,066 (Lorenzo, Aug. 25, 2020, “Aneurysm Device and Delivery System”) disclose a self-expanding braided tubular member. U.S. patent application 20190053811 (Garza et al., Feb. 21, 2019, “Flow Attenuation Device”) and U.S. Pat. No. 11,071,551 (Garza et al., Jul. 27, 2021, “Flow Attenuation Device”) disclose an embolic device for treating aneurysms with a desired porosity only at discrete sections. U.S. patent application 20190192168 (Lorenzo et al., Jun. 27, 2019, “Aneurysm Device and Delivery Method”) and U.S. Pat. No. 10,716,574 (Lorenzo et al., Jul. 21, 2020, “Aneurysm Device and Delivery Method”) discloses a self-expanding braid for treating an aneurysm, including a method for inverting and buckling a proximal segment.

U.S. patent application 20190223878 (Lorenzo et al., Jul. 25, 2019, “Aneurysm Device and Delivery System”) and U.S. patent application 20200397447 (Lorenzo et al., Dec. 24, 2020, “Aneurysm Device and Delivery System”) discloses an expandable segment which radially expands inside an outer occlusive sack. U.S. patent application 20190223881 (Hewitt et al., Jul. 25, 2019, “Devices for Therapeutic Vascular Procedures”) discloses a self-expanding resilient permeable shell made from elongate resilient filaments with a distal region that extends beyond the distal end of the permeable shell. U.S. Pat. No. 10,398,441 (Warner et al., Sep. 3, 2019, “Vascular Occlusion”) discloses a vascular treatment system with a containment device, a pusher, and a stopper ring. U.S. patent application 20190343532 (Divino et al., Nov. 14, 2019, “Occlusive Devices”) discloses a device with at least one expandable structure which is adapted to transition from a compressed configuration to an expanded configuration when released into an aneurysm.

U.S. Pat. No. 10,478,194 (Rhee et al., Nov. 19, 2019, “Occlusive Devices”) and U.S. patent application 20200038032 (Rhee et al., Feb. 6, 2020, “Occlusive Devices”) disclose an implant with a frame and a mesh component, wherein the mesh component has a first porosity and the frame has a second porosity. U.S. patent application 20190365385 (Gorochow et al., Dec. 5, 2019, “Aneurysm Device and Delivery System”) and U.S. Pat. No. 10,939,915 (Gorochow et al., Mar. 9, 2021, “Aneurysm Device and Delivery System”) discloses a braid, wherein translating the braid causes a delivery portion to expand and form a distal sack as well as invert into itself. U.S. patent application 20200029973 (Walzman, Jan. 30, 2020, “Mash Cap for Ameliorating Outpouchings”) discloses an embolic device comprising a control element, a catheter element, a delivery microcatheter hypotube, a detachment element, a mesh disc, a distal opening, and at least one attached extension arm.

U.S. patent application 20200038034 (Maguire et al., Feb. 6, 2020, “Vessel Occluder”) discloses a vessel occluder with an expandable mesh portion having a flexible membrane that expands within a cavity of the expandable mesh portion. U.S. Pat. No. 10,610,231 (Marchand et al., Apr. 7, 2020, “Filamentary Devices for Treatment of Vascular Defects”) discloses a self-expanding resilient permeable shell with a plurality of elongate resilient filaments with a woven structure, wherein the plurality of filaments includes small filaments and large filaments, and wherein the small filaments have a transverse dimension which is smaller than the transverse dimension of the large filaments.

U.S. patent application 20200113576 (Gorochow et al., Apr. 16, 2020, “Folded Aneurysm Treatment Device and Delivery Method”) and U.S. patent application 20210196284 (Gorochow et al., Jul. 1, 2021, “Folded Aneurysm Treatment Device and Delivery Method”) disclose an implant having a braided section that folds to form an outer occlusive sack extending across a neck of an aneurysm to engage a wall of the aneurysm from within a sac of the aneurysm and an inner occlusive sack forming a trough nested within the outer occlusive sack.

U.S. Pat. No. 10,653,425 (Gorochow et al., May 19, 2020, “Layered Braided Aneurysm Treatment Device”), U.S. patent application 20200367893 (Xu et al., Nov. 26, 2020, “Layered Braided Aneurysm Treatment Device”), U.S. patent application 20200367898 (Gorochow et al., Nov. 26, 2020, “Layered Braided Aneurysm Treatment Device”), U.S. Pat. No. 11,413,046 (Xu et al., Aug. 16, 2022, “Layered Braided Aneurysm Treatment Device”), and U.S. patent application 20200367900 (Pedroso et al., Nov. 26, 2020, “Layered Braided Aneurysm Treatment Device With Corrugations”) disclose a tubular braid comprising an open end, a pinched end, and a predetermined shape; wherein, in the predetermined shape, the tubular braid comprises: a first segment extending from the open end to a first inversion, a second segment encircled by the open end such that the second segment is only partially surrounded by the first segment and extending from the first inversion to a second inversion, and a third segment surrounded by the second segment and extending from the second inversion to the pinched end.

U.S. patent application 20200155333 (Franano et al., May 21, 2020, “Ballstent Device and Methods of Use”) discloses a rounded, thin-walled, expandable metal structure and a flexible, elongated delivery device. U.S. patent application 20200187952 (Walsh et al., Jun. 18, 2020, “Intrasaccular Flow Diverter for Treating Cerebral Aneurysms”) and U.S. patent application 20220151632 (Walsh et al., May 19, 2022, “Intrasaccular Flow Diverter for Treating Cerebral Aneurysms”) disclose a stabilizing frame with two parts, the first part sized to anchor within the sac of the aneurysm and the exterior part sized to anchor against a region of the blood vessel wall adjacent the aneurysm neck.

U.S. patent application 20200187953 (Hamel et al., Jun. 18, 2020, “Devices, Systems, and Methods for the Treatment of Vascular Defects”) discloses a mesh comprising a first end portion, a second end portion, and a length extending between the first and second end portions, and a first lateral edge, a second lateral edge, and a width extending between the first and second lateral edges. U.S. patent application 20200205841 (Aboytes et al., Jul. 2, 2020, “Devices, Systems, and Methods for the Treatment of Vascular Defects”) and U.S. patent application 20210378681 (Aboytes et al., Dec. 9, 2021, “Devices, Systems, and Methods for the Treatment of Vascular Defects”) disclose aneurysm occlusion devices with a first configuration in which a first portion and a second portion are substantially linearly aligned and a second configuration in which the second portion at least partially overlaps the first portion.

U.S. patent application 20200281603 (Marchand et al., Sep. 10, 2020, “Filamentary Devices for Treatment of Vascular Defects”) discloses a permeable shell including a really swell polymer. U.S. patent application 20200289125 (Dholakia et al., Sep. 17, 2020, “Filamentary Devices Having a Flexible Joint for Treatment of Vascular Defects”) discloses an implant with a first permeable shell having a proximal end with a concave or recessed section and a second permeable shell having a convex section that mates with the concave or recessed section. U.S. patent application 20200289126 (Hewitt et al., Sep. 17, 2020, “Filamentary Devices for Treatment of Vascular Defects”), U.S. Pat. No. 11,317,921 (Hewitt et al., May 3, 2022, “Filamentary Devices for Treatment of Vascular Defects”), and U.S. patent application 20220257258 (Hewitt et al., Aug. 18, 2022, “Filamentary Devices for Treatment of Vascular Defects”) disclose a permeable shell or mesh with a stiffer proximal portion at the neck of an aneurysm.

U.S. patent application 20200305885 (Soto Del Valle et al, Oct. 1, 2020, “Aneurysm Treatment Device”) discloses an occlusion device that expands to form a cup shape within an aneurysm sac. U.S. patent application 20200305886 (Soto Del Valle et al, Oct. 1, 2020, “Aneurysm Treatment Device”) and U.S. patent application 20220225997 (Soto Del Valle et al., Jul. 21, 2022, “Aneurysm Treatment Device”) disclose a device with an expandable sack with a free open end and an elongated looping portion. U.S. patent application 20200367896 (Zaidat et al., Nov. 26, 2020, “Systems and Methods for Treating Aneurysms”) discloses an apparatus for treating an aneurysm in a blood vessel with a first tubular mesh having a first end and a second end coupled together at a proximal end of the occlusion element.

U.S. patent application 20200367904 (Becking et al., Nov. 26, 2020, “Multiple Layer Filamentary Devices for Treatment of Vascular Defects”) and U.S. patent application 20220022886 (Becking et al., Jan. 27, 2022, “Multiple Layer Filamentary Devices for Treatment of Vascular Defects”) disclose braid-balls suitable for aneurysm occlusion. U.S. patent application 20200367906 (Xu et al., Nov. 26, 2020, “Aneurysm Treatment With Pushable Ball Segment”) and U.S. patent application 20230016312 (Xu et al., Jan. 19, 2023, “Aneurysm Treatment with Pushable Implanted Braid”) disclose a braided implant with a retractable dual proximal layer. U.S. patent application 20200375606 (Lorenzo, Dec. 3, 2020, “Aneurysm Method and System”) discloses a braided implant which is invertible about the distal implant end.

U.S. patent application 20200375607 (Soto Del Valle et al., Dec. 3, 2020, “Aneurysm Device and Delivery System”) discloses a method of expanding mesh segments to form an outer occlusive sack and an inner occlusive sack. U.S. patent application 20200405347 (Walzman, Dec. 31, 2020, “Mesh Cap for Ameliorating Outpouchings”) discloses a self-expandable occluding device which covers the neck of an outpouching and serves as a permanent embolic plug. U.S. patent application 20210007755 (Lorenzo et al., Jan. 14, 2021, “Intrasaccular Aneurysm Treatment Device with Varying Coatings”) discloses an aneurysm intrasaccular implant with coated regions. U.S. Pat. No. 10,905,430 (Lorenzo et al., Feb. 2, 2021, “Aneurysm Device and Delivery System”) discloses an expandable segment which radially expands inside an outer occlusive sack.

U.S. patent application 20210052279 (Porter et al., Feb. 25, 2021, “Intra-Aneurysm Devices”) discloses a device including an upper member that sits against the dome of an aneurysm, a lower member that sits in the neck of the aneurysm, and a means of adjusting the overall dimensions of the device. U.S. patent application 20210085333 (Gorochow et al., Mar. 25, 2021, “Inverting Braided Aneurysm Treatment System and Method”), U.S. Pat. No. 11,278,292 (Gorochow et al., Mar. 22, 2022, “Inverting Braided Aneurysm Treatment System and Method”), and U.S. patent application 20220104829 (Gorochow et al., Apr. 7, 2022, “Inverting Braided Aneurysm Treatment System and Method”) disclose a tubular braid with an intrasaccular section, an intravascular section, a pinched section, and a predetermined shape. U.S. patent application 20210106338 (Gorochow, Apr. 15, 2021, “Spiral Delivery System for Embolic Braid”) discloses a braided implant having a spiral segment.

U.S. patent application 20210128160 (Li et al., May 6, 2021, “Systems and Methods for Treating Aneurysms”) discloses delivering an occlusive member to an aneurysm sac in conjunction with an embolic element. U.S. patent application 20210128160 (Li et al., May 6, 2021, “Systems and Methods for Treating Aneurysms”), U.S. patent application 20210128167 (Patel et al., May 6, 2021, “Systems and Methods for Treating Aneurysms”), U.S. patent application 20210128168 (Nguyen et al., May 6, 2021, “Systems and Methods for Treating Aneurysms”) and disclose delivering an occlusive member (e.g., an expandable braid) to an aneurysm sac in conjunction with an embolic element (e.g., coils, embolic material).

U.S. patent application 20210128161 (Nageswaran et al., May 6, 2021, “Aneurysm Treatment Device”) discloses an aneurysm treatment system including a conduit with a distal portion, a coupler slidably coupled to the distal portion, an occlusive member coupled to the coupler, and a securing member coupled to the conduit proximal to the coupler. U.S. patent application 20210128162 (Rhee et al., May 6, 2021, “Devices, Systems, and Methods for Treatment of Intracranial Aneurysms”) discloses introduction of an embolic element to a space between an occlusive member and an inner surface of the aneurysm wall. U.S. patent application 20210128162 (Rhee et al., May 6, 2021, “Devices, Systems, and Methods for Treatment of Intracranial Aneurysms”) discloses delivering an occlusive member to an aneurysm cavity and transforming a shape of the occlusive member within the cavity, including delivering an embolic element between the occlusive member and the aneurysm wall.

U.S. patent application 20210128165 (Pulugurtha et al., May 6, 2021, “Systems and Methods for Treating Aneurysms”) discloses an occlusive member configured to be positioned within an aneurysm sac and a distal conduit coupled to the occlusive member and having a first lumen extending therethrough. U.S. patent application 20210128165 (Pulugurtha et al., May 6, 2021, “Systems and Methods for Treating Aneurysms”) and U.S. Pat. No. 11,305,387 (Pulugurtha et al., Apr. 19, 2022, “Systems and Methods for Treating Aneurysms”) disclose a distal conduit coupled to an occlusive member with a first lumen extending therethrough and a proximal conduit with a second lumen extending therethrough. U.S. patent application 20210128167 (Patel et al., May 6, 2021, “Systems and Methods for Treating Aneurysms”) discloses delivering an occlusive member to an aneurysm sac in conjunction with an embolic element.

U.S. patent applications 20210128168 (Nguyen et al., May 6, 2021, “Systems and Methods for Treating Aneurysms”) and 20230023511 (Nguyen et al., Jan. 26, 2023, “Systems and Methods for Treating Aneurysms”) disclose delivering an occlusive member to an aneurysm sac in conjunction with an embolic element. U.S. patent application 20210128169 (Li et al., May 6, 2021, “Devices, Systems, and Methods for Treatment of Intracranial Aneurysms”) and U.S. patent application 20210153872 (Nguyen et al., May 27, 2021, “Devices, Systems, and Methods for Treatment of Intracranial Aneurysms”) disclose delivering an occlusive member to an aneurysm cavity and deforming a shape of the occlusive member via introduction of an embolic element to a space between the occlusive member and an inner surface of the aneurysm wall.

U.S. patent applications 20210128169 (Li et al., May 6, 2021, “Devices, Systems, and Methods for Treatment of Intracranial Aneurysms”), 20210153872 (Nguyen et al., May 27, 2021, “Devices, Systems, and Methods for Treatment of Intracranial Aneurysms”), 20230294223 (Li et al., Sep. 21, 2023, “Devices, Systems, and Methods for Treatment of Intracranial Aneurysms”), and 20230373040 (Nguyen et al., Nov. 23, 2023, “Devices, Systems, and Methods for Treatment of Intracranial Aneurysms”) disclose delivering an occlusive member to an aneurysm cavity and deforming a shape of the occlusive member via introduction of an embolic element to a space between the occlusive member and the aneurysm wall. U.S. patent application 20210129275 (Nguyen et al., May 6, 2021, “Devices, Systems, and Methods for Treating Aneurysms”) discloses methods of manufacturing an occlusive device including conforming a mesh to a forming assembly and setting a shape of the mesh based on the forming assembly.

U.S. patent applications 20210129275 (Nguyen et al., May 6, 2021, “Devices, Systems, and Methods for Treating Aneurysms”) and 20230311254 (Nguyen et al., Oct. 5, 2023, “Devices, Systems, and Methods for Treating Aneurysms”) disclose methods of manufacturing an aneurysm occlusion device including shaping a mesh with a forming assembly comprising multiple forming members, a mandrel, and/or one or more coupling elements. U.S. patent application 20210137526 (Lee et al., May 13, 2021. “Embolic Devices for Occluding Body Lumens”) discloses an embolic device with a first segment forming a first three-dimensional structure, wherein the first three-dimensional structure defines a cavity; and a second segment forming a second three-dimensional structure; wherein the cavity of the first three-dimensional structure is configured to accommodate at least a majority of the second three-dimensional structure.

U.S. patent application 20210145449 (Gorochow, May 20, 2021, “Implant Delivery System with Braid Cup Formation”) discloses an implant system with an engagement wire, a pull wire, and a braided implant having a distal ring thereon. U.S. Pat. No. 11,013,516 (Franano et al., May 25, 2021, “Expandable Body Device and Method of Use”) discloses a single-lobed, thin-walled, expandable body comprising gold, platinum, or silver. U.S. patent application 20210169495 (Gorochow et al., Jun. 10, 2021, “Intrasaccular Inverting Braid with Highly Flexible Fill Material”) and U.S. Pat. No. 11,602,350 (Gorochow et al., Mar. 14, 2023, “Intrasaccular Inverting Braid with Highly Flexible Fill Material”) disclose a tubular braided implant which is delivered as a single layer braid, inverted into itself during deployment to form at least two nested sacks and includes additional braid material that can fill the innermost sack.

U.S. patent application 20210169498 (Gorochow, Jun. 10, 2021, “Delivery of Embolic Braid”) discloses a braided implant delivery system which attaches a braided implant having a band to a delivery tube, positions the braided implant within an aneurysm, and then releases the band from the delivery tube. U.S. Pat. No. 11,033,275 (Franano et al., Jun. 15, 2021, “Expandable Body Device and Method of Use”) discloses devices, designs, methods of manufacturing and using hollow gold structures that can be folded, wrapped, and compressed. U.S. patent application 20210177429 (Lorenzo, Jun. 17, 2021, “Aneurysm Method and System”) discloses a vaso-occlusive device with at least two nested sacks.

U.S. patent application 20210186518 (Gorochow et al., Jun. 24, 2021, “Implant Having an Intrasaccular Section and Intravascular Section”) and U.S. Pat. No. 11,457,926 (Gorochow et al., Oct. 4, 2022. “Implant Having an Intrasaccular Section and Intravascular Section”) disclose a tubular braid with an intrasaccular section, an intravascular section, a pinched section, and a predetermined shape. U.S. Pat. No. 11,051,825 (Gorochow, Jul. 6, 2021, “Delivery System for Embolic Braid”) discloses a braided implant which is attached to a releasing component that can be detachably engaged with a delivery tube and a pull wire. U.S. Pat. No. 11,058,430 (Gorochow et al., Jul. 13, 2021, “Aneurysm Device and Delivery System”) discloses a braid with a proximal expandable portion for positioning inside an aneurysm and sealing across the neck of the aneurysm.

U.S. Pat. No. 11,058,431 (Pereira et al., Jul. 13, 2021, “Systems and Methods for Treating Aneurysms”) discloses an inverted mesh tube having an outer layer and an inner layer, wherein the outer layer transitions to the inner layer at an inversion fold located at or adjacent to the distal end of the occlusion element. U.S. patent application 20210228214 (Bowman et al., Jul. 29, 2021, “Devices for Vascular Occlusion”) discloses a method of using and delivering an occlusive device. U.S. Pat. No. 11,076,861 (Gorochow et al., Aug. 3, 2021, “Folded Aneurysm Treatment Device and Delivery Method”) discloses an implant with a fold which defines an annular ridge and a radiopaque marker band.

U.S. patent application 20210244420 (Aboytes et al., Aug. 12, 2021, “Devices and Methods for the Treatment of Vascular Defects”) discloses aneurysm occlusion devices with a first configuration in which a first portion and a second portion are substantially linearly aligned and a second configuration in which the second portion at least partially overlaps the first portion. U.S. patent application 20210275187 (Franano et al., Sep. 9, 2021, “Expandable Body Device and Method of Use”) discloses medical devices comprising a single-lobed, thin-walled, expandable body. U.S. patent application 20210275779 (Northrop, Sep. 9, 2021, “Actuating Elements for Bending Medical Devices”) discloses an actuating element causes a tube to bend.

U.S. patent application 20210282784 (Sepetka et al., Sep. 16, 2021, “Occlusive Device”) discloses a device comprising a plurality of braided wires and an embolic coil. U.S. patent application 20210282785 (Dholakia et al., Sep. 16, 2021, “Devices Having Multiple Permeable Shells for Treatment of Vascular Defects”) a device with a plurality of permeable shells connected by a plurality of coils. U.S. patent application 20210282789 (Vu et al., Sep. 16, 2021, “Multiple Layer Devices for Treatment of Vascular Defects”) discloses a first permeable shell and a second permeable shell, where the second permeable shell sits within an interior cavity of the first permeable shell. U.S. Pat. No. 11,123,077 (Lorenzo et al., Sep. 21, 2021, “Intrasaccular Device Positioning and Deployment System”) discloses implant deployment systems including a braided implant that can be detachably attached to a delivery tube by an expansion ring.

U.S. patent application 20210330331 (Lorenzo, Oct. 28, 2021, “Aneurysm Occlusion Device”) and U.S. Pat. No. 11,154,302 (Lorenzo et al., Oct. 26, 2021, “Aneurysm Occlusion Device”) disclose an occlusion device with a substantially annular body disposed on the proximal end region of the device. U.S. patent application 20210338247 (Gorochow, Nov. 4, 2021, “Double Layer Braid”) discloses a double layered braid for treating an aneurysm. U.S. patent application 20210338250 (Gorochow et al., Nov. 4, 2021, “Intrasaccular Flow Diverter”) and U.S. Pat. No. 11,523,831 (Gorochow et al., Dec. 13, 2022, “Intrasaccular Flow Diverter”) disclose an interior fill braid physically which is inverted over itself to form a proximal inverted end and an opposite free end and a dome braid disposed distally of and secured to the interior fill braid.

U.S. Pat. No. 11,166,731 (Wolfe et al., Nov. 9, 2021, “Systems and Methods for Treating Aneurysms”) discloses an inverted mesh tube having an outer layer and an inner layer, the outer layer transitioning to the inner layer at an inversion fold. U.S. patent application 20210346032 (Patterson et al., Nov. 11, 2021, “Devices for Treatment of Vascular Defects”) discloses an expandable stent for placement in a parent vessel proximal, near, or adjacent an aneurysm. U.S. patent application 20210353299 (Hamel et al., Nov. 18, 2021, “Devices, Systems, and Methods for the Treatment of Vascular Defects”) discloses a mesh that is curved along its length with an undulating contour across at least a portion of one or both of its length or its width.

U.S. patent application 20210353300 (Kottenmeier et al., Nov. 18, 2021, “Systems and Methods for Treatment of Defects in the Vasculature”) discloses aneurysm occlusion methods and systems including an expandable stent. U.S. Pat. No. 11,179,159 (Cox et al., Nov. 23, 2021, “Methods and Devices for Treatment of Vascular Defects”) discloses a device comprising a first hub, a second hub, a support structure having a longitudinal axis, the support structure disposed between the first hub and the second hub, the support structure including a plurality of struts, and a layer of material disposed over the plurality of struts, wherein the first hub is cylindrical and connected to an end of each of the struts of the plurality of struts. U.S. Pat. No. 11,185,335 (Badruddin et al., Nov. 30, 2021, “System for and Method of Treating Aneurysms”) discloses an apparatus for treating an aneurysm with an occlusion element disposed on a wire, wherein the occlusion element includes a cover for covering a neck of an aneurysm and an inner anchoring member.

U.S. Pat. No. 11,202,636 (Zaidat et al., Dec. 21, 2021, “Systems and Methods for Treating Aneurysms”), U.S. patent application 20220022884 (Wolfe et al., Jan. 27, 2022, “Systems and Methods for Treating Aneurysms”), and U.S. patent application 20220211383 (Pereira et al., Jul. 7, 2022, “Systems and Methods for Treating Aneurysms”) disclose an apparatus for treating an aneurysm including an occlusion element configured to be releasably coupled to an elongate delivery shaft and a distal end, a proximal end, and a longitudinal axis extending between the distal end and the proximal end. U.S. patent application 20220031334 (Aguilar, Feb. 3, 2022, “Expandable Devices for Treating Body Lumens”) discloses an occlusive device comprising an expandable mesh including an outer mesh and an inner mesh disposed within the outer mesh, a connection portion positioned at or within an inner cavity of the inner mesh and configured to be detachably coupled to a delivery member.

U.S. patent application 20220031334 (Aguilar, Feb. 3, 2022, “Expandable Devices for Treating Body Lumens”) discloses an occlusive device comprising an expandable mesh including an outer mesh and an inner mesh disposed within the outer mesh. U.S. patent application 20220039804 (Rangwala et al., Feb. 10, 2022, “Flow-Diverting Implant and Delivery Method”) discloses a saddle-shaped braided mesh diverter that covers the neck of an aneurysm. U.S. patent application 20220054141 (Zaidat et al., Feb. 24, 2022, “Systems and Methods for Treating Aneurysms”) discloses an apparatus for treating an aneurysm in a blood vessel with a first tubular mesh having a first end and a second end coupled together at a proximal end of the occlusion element.

U.S. patent application 20220087681 (Xu et al., Mar. 24, 2022, “Inverting Braided Aneurysm Implant with Dome Feature”) discloses an implant with a dome feature configured to press into aneurysm walls near the aneurysm's dome and facilitate securement of the braid across the aneurysm's neck. U.S. Pat. No. 11,337,706 (Soto Del Valle et al., May 24, 2022, “Aneurysm Treatment Device”) discloses an implant having an elongated portion and an expandable braided sack portion. U.S. patent application 20220175389 (Wallace et al., Jun. 9, 2022, “Vaso-Occlusive Devices Including a Friction Element”) discloses a vaso-occlusive implant with a friction element between a soft braided member and a coil.

U.S. patent application 20220192678 (Hewitt et al., Jun. 23, 2022, “Filamentary Devices for Treatment of Vascular Defects”) discloses an implant having a first permeable shell having a proximal hub and an open distal end and a second permeable shell having a distal hub and an open proximal end. U.S. patent application 20220202425 (Gorochow et al., Jun. 30, 2022, “Semispherical Braided Aneurysm Treatment System and Method”) discloses a tubular braid with three segments and two inversions, one of the three segments extending between the two inversions and forming a sack. U.S. patent application 20220249098 (Milhous et al., Aug. 11, 2022, “Filamentary Devices for Treatment of Vascular Defects”) discloses a permeable implant with a plurality of scaffolding filaments. U.S. patent application 20220257260 (Hewitt et al., Aug. 18, 2022, “Filamentary Devices for Treatment of Vascular Defects”) discloses an implant having multiple mesh layers.

U.S. Pat. No. 11,426,175 (Morita et al., Aug. 30, 2022, “Expansile Member”) discloses an occlusive system comprising: a catheter; a shell deliverable through the catheter, a delivery pusher detachably connected to the shell and configured to navigate the shell through the catheter, wherein the shell has a globular shaped portion. U.S. Pat. No. 11,471,162 (Griffin, Oct. 18, 2022, “Occlusion Device”) discloses an occlusion device for implantation into a body lumen or aneurysm which has a continuous compressible mesh structure comprising axial mesh carriages configured end to end, wherein each end of each carriage is a pinch point in the continuous mesh structure. U.S. patent application 20220330947 (Henkes et al., Oct. 20, 2022, “Implant for the Treatment of Aneurysms”) discloses an implant which is rolled up relative to a radial axis in order to form a balled-up configuration.

U.S. Pat. No. 11,497,504 (Xu et al., Nov. 15, 2022, “Aneurysm Treatment with Pushable Implanted Braid”) discloses a braided implant with a retractable dual proximal layer. U.S. Pat. No. 11,498,165 (Patel et al., Nov. 15, 2022, “Systems and Methods for Treating Aneurysms”) discloses an occlusive implant with a conduit which receives a liquid embolic. U.S. patent application 20220370078 (Chen et al., Nov. 24, 2022, “Vaso-Occlusive Devices”) discloses a vaso-occlusive structure made with a gold-platinum-tungsten alloy. U.S. Pat. No. 11,517,321 (Mauger et al., Dec. 6, 2022, “System and Methods for Embolized Occlusion of Neurovascular Aneurysms”) discloses an occlusion device with a reinforcing portion with no porosity. U.S. patent application 20230017150 (Lee et al., Jan. 19, 2023, “Hydrogel Stent and Embolization Device for Cerebral Aneurysm”) discloses a hydrogel stent for occluding a cerebral aneurysm.

U.S. Pat. No. 11,559,309 (Rangwala et al., Jan. 24, 2023, “Filamentary Devices for Treatment of Vascular Defects”) discloses a permeable implant whose proximal portion is stiffer. U.S. patent application 20230031965 (Sivapatham, Feb. 2, 2023, “Intrasaccular Stent Device for Aneurysm Treatment”) discloses a system for treating an aneurysm in a blood vessel comprising a catheter, a guidewire, a delivery wire, an intrasaccular stent/retaining device removably attached to the delivery wire, and endovascular coiling. U.S. patent application 20230039246 (Hossan et al., Feb. 9, 2023, “Non-Braided Biodegradable Flow Diverting Device for Endovascular Treatment of Aneurysm and Associated Fabrication Method”) discloses a biodegradable flow-diverting device that regulates blood flow into an aneurysmal sac.

U.S. patent application 20230039773 (Monstadt et al., Feb. 9, 2023, “Implant for Treating Aneurysms”) discloses an implant which is preset to a specific structure. U.S. Pat. No. 11,583,282 (Gorochow et al., Feb. 21, 2023, “Layered Braided Aneurysm Treatment Device”) discloses a method for shaping a tubular braid into a predetermined shape. U.S. Pat. No. 11,589,872 (Mauger, Feb. 28, 2023, “Vascular Occlusion Devices Utilizing Thin Film Nitinol Foils”) discloses an implantable occlusion device wherein mesh components are wrapped around a support structure and slot that enables a disc to be wrapped around the support structure with overlapping portions. U.S. patent application 20230061363 (Yee et al., Mar. 2, 2023, “Embolic Device with Improved Neck Coverage”) discloses an embolic device with a flexible structure which has a series of alternating narrow portions and link portions.

U.S. Pat. No. 11,596,412 (Xu et al., Mar. 7, 2023, “Aneurysm Device and Delivery System”) discloses a braid with a proximal portion which goes across an aneurysm neck and an expandable distal portion. U.S. Pat. No. 11,607,226 (Pedroso et al., Mar. 21, 2023, “Layered Braided Aneurysm Treatment Device with Corrugations”) discloses an implant with a proximal inversion and two segments. U.S. patent application 20230107778 (Cox et al., Apr. 6, 2023, “Methods and Devices for Treatment of Vascular Defects”) discloses an expandable body comprising a plurality of elongate filamentary elements each having a first end and a second end, wherein the elements extend from a first end of the device to a second end of the device and back to the first end of the device.

U.S. patent application 20230114169 (Hewitt et al., Apr. 13, 2023, “Devices for Treatment of Vascular Defects”) discloses a permeable woven implant with a radially-constrained state for delivery within a catheter and an expanded state thereafter. U.S. patent application 20230157696 (Carrillo, May 25, 2023, “Aneurysm Treatment Device and Associated Systems and Methods of Use”) discloses an aneurysm treatment device with a tip portion, a body portion, and a base portion. U.S. patent application 20230165587 (Carrillo, Jun. 1, 2023, “Expandable Devices for Treating Body Lumens”) discloses an expandable cage with a plurality of mesh stents which receives embolic material therein. U.S. patent application 20230190292 (Choubey et al., Jun. 22, 2023, “Occlusive Devices with Petal-Shaped Regions for Treating Vascular Defects”) discloses an occlusive device for treating an aneurysm with a mesh formed from a tubular braid, including a petal-shaped region formed from a flattened section of the tubular braid.

U.S. Pat. No. 11,685,007 (Li et al., Jun. 27, 2023, “Devices, Systems, and Methods for Treatment of Intracranial Aneurysms”) discloses delivering an occlusive member to an aneurysm cavity and deforming a shape of the occlusive member via introduction of an embolic element to a space between the occlusive member and the aneurysm wall. U.S. patent application 20230225735 (Kulak et al., Jul. 20, 2023, “Expandable Devices for Treating Body Lumens”) discloses an occlusive device comprising a mesh having a low-profile state for intravascular delivery to the aneurysm and a deployed state, wherein the mesh comprises a tubular mesh configured to curve along its longitudinal dimension when implanted in an aneurysm cavity. U.S. patent application 20230225735 (Kulak et al., Jul. 20, 2023, “Expandable Devices for Treating Body Lumens”) discloses a tubular mesh which curves along its longitudinal dimension when implanted in an aneurysm cavity.

U.S. patent application 20230240686 (Ashby et al., Aug. 3, 2023, “Occlusive Devices with Spiral Struts for Treating Vascular Defects”) discloses an occlusive device with a plurality of spiral struts. U.S. patent application 20230252631 (Kashyap et al., Aug. 10, 2023, “Neural Network Apparatus for Identification, Segmentation, and Treatment Outcome Prediction for Aneurysms”) discloses using medical imaging and a neural network to predict outcomes from the potential use of one or more different intrasaccular implant devices. U.S. patent application 20230263528 (Jones, Aug. 24, 2023, “Intrasacular Flow Diverter and Related Methods”) discloses an intrasaccular flow diverter with a plurality of wires which are coiled to form a collapsible, substantially spherical frame.

U.S. patent application 20230277184 (Rashidi et al., Sep. 7, 2023, “Occlusive Devices with Thrombogenic Inserts”) discloses an expandable mesh which spans a neck of the aneurysm with an insert configured to promote thrombosis. U.S. patent application 20230277184 (Rashidi, Sep. 7, 2023, “Occlusive Devices With Thrombogenic Inserts”) discloses an occlusive device for treating an aneurysm includes an expandable mesh configured to span a neck of the aneurysm, wherein expandable mesh can include an upper wall, a lower wall, and an insert between the upper and lower walls. U.S. patent application 20230285031 (Mayer et al, Sep. 14, 2023, “Device for Restricting Blood Flow to Aneurysms”) discloses a non-occlusive device with a coilable section and a docking section.

U.S. patent application 20230301660 (Sonmez et al., Sep. 28, 2023, “Systems and Methods for Treating Aneurysms”) discloses delivering an occlusive member to an aneurysm sac in conjunction with an embolic element. U.S. patent application 20230397913 (Greenwood et al., Dec. 14, 2023, “Friction Fit Endovascular Implant Detachment Mechanism”) discloses an endovascular implant including an open end and a pinched end, a connector positioned approximate the pinched end, a lock wire, and an outer coil surrounding the lock wire. U.S. patent application 20230404590 (Xu et al., Dec. 21, 2023, “Inverting Braided Aneurysm Treatment System Having a Semi-Frustoconically-Shaped Portion”) discloses a tubular braid with a first segment extending from an open end to a first inversion, a second segment extending from the first inversion to a second inversion, and a third segment which extends from the second inversion to a pinched end.

U.S. patent application 20230404592 (Nageswaran, Dec. 21, 2023, “Implant with Intrasaccular and Intravascular Portions and Related Technology”) discloses technology configured for treating an aneurysm at a treatment location within a patient's vasculature at which first, second, and third blood vessels converge. U.S. patent application 20240016499 (Xu et al., Jan. 18, 2024, “Braided Implant with Atraumatic End”) discloses a tubular braid including an open end and a pinched end. U.S. patent application 20240032941 (Shimizu et al., Feb. 1, 2024, “Embolic Material Delivery Device and Related Technology”) discloses an elongate conduit body defining an axial lumen through which the conduit body is configured to convey liquid embolic material toward the aneurysm. U.S. patent application 20240050099 (Pecor et al., Feb. 15, 2024, “Occlusive Devices for Treating Vascular Defects and Associated Systems and Methods”) discloses a plurality of braided filaments configured to be implanted in an aneurysm cavity.

U.S. patent application 20240065699 (Sloss et al., Feb. 29, 2024, “Twister Implant Detachment Mechanism”) discloses an endovascular implant detachment mechanism with a connector, a push wire, and one or more lock wires. U.S. patent application 20240075565 (Li et al., Mar. 7, 2024, “Systems and Methods for Treating Aneurysms”) discloses delivering an occlusive member to an aneurysm sac in conjunction with an embolic element. U.S. patent application 20240099720 (Greenwood et al., Mar. 28, 2024, “Braided Implant with Detachment Mechanism”) discloses an occlusive device with a segment including an open end and a proximal end, as well as a push wire that is positioned proximal of the proximal end. U.S. patent application 20240108354 (Xu et al., Apr. 4, 2024, “Braided Implant with Integrated Embolic Coil”) discloses a tubular braid with a band positioned near a pinched end which is attached to an embolic coil.

### SUMMARY OF THE INVENTION

Disclosed herein is an intrasaccular aneurysm occlusion device with a convex distal portion and a concave proximal portion. When deployed, the convex distal portion is at least partially nested in the concave proximal portion. In an example, the convex distal portion can have a generally globular shape. In an example, the convex distal portion can have a heart (e.g. limacon and/or cardioid) shape. In an example, the convex distal portion can be a flexible net or mesh. In an example, the concave proximal portion can be bowl-shaped. In an example, the convex distal portion and the concave proximal portion can be made from a single tubular mesh. In an example, the convex distal portion and the concave proximal portion can be made by radially-constraining, inverting, and/or everting a tubular mesh.

In another example, a convex distal portion and a concave proximal portion can be made separately from different materials and/or different processes. In an example, a convex distal portion and a concave proximal portion can made separately and connected before insertion into an aneurysm sac. In another example, a convex distal portion and a concave proximal portion can be made connected within an aneurysm sac. In an example, a convex distal portion can be a flexible polymer net or mesh and a concave proximal portion can be made by braiding or weaving wires. In an example, a convex distal portion can be expanded by being filled with embolic members and/or material, such as embolic coils or “string-of-pearls” embolic strands.

### BRIEF INTRODUCTION TO THE FIGURES

FIGS. 1 through 6 show an aneurysm occlusion device with a globular distal portion and a bowl-shaped proximal portion.

FIG. 1 shows this device without a radial constraint.

FIG. 2 shows this device with a radial constraint.

FIG. 3 shows this device wherein the entire distal portion is nested in the proximal portion.

FIG. 4 shows this device with embolic coils in the distal portion.

FIG. 5 shows this device with “string-of-pearls” embolic strands in the distal portion.

FIG. 6 shows this device with congealing liquid in the distal portion.

FIGS. 7 through 12 show an aneurysm occlusion device with a bowl-shaped distal portion and a bowl-shaped proximal portion.

FIG. 7 shows this device without a radial constraint.

FIG. 8 shows this device with a radial constraint.

FIG. 9 shows this device wherein the entire distal portion is nested in the proximal portion.

FIG. 10 shows this device with embolic coils in the distal portion.

FIG. 11 shows this device with “string-of-pearls” embolic strands in the distal portion.

FIG. 12 shows this device with congealing liquid in the distal portion.

FIGS. 13 through 18 show an aneurysm occlusion device with a heart-shaped (e.g. cardioid) distal portion and a bowl-shaped proximal portion.

FIG. 13 shows this device without a radial constraint.

FIG. 14 shows this device with a radial constraint.

FIG. 15 shows this device wherein the entire distal portion is nested in the proximal portion.

FIG. 16 shows this device with embolic coils in the distal portion.

FIG. 17 shows this device with “string-of-pearls” embolic strands in the distal portion.

FIG. 18 shows this device with congealing liquid in the distal portion.

FIGS. 19 through 22 show an intrasaccular aneurysm occlusion device with a flexible distal net or mesh and a bowl-shaped proximal portion.

FIG. 19 shows this device without a radial constraint.

FIG. 20 shows this device with a radial constraint.

FIG. 21 shows this device with embolic coils in the flexible net or mesh.

FIG. 22 shows this device with “string-of-pearls” embolic strands in the flexible net or mesh.

### DETAILED DESCRIPTION OF THE FIGURES

FIGS. 1 through 6 show an example of an intrasaccular aneurysm occlusion device comprising: a convex distal portion which is inserted into an aneurysm sac; and a concave proximal portion which is inserted into the aneurysm sac and expands to cover the aneurysm neck; wherein the convex distal portion is between the concave proximal portion and the dome of the aneurysm sac, wherein at least part of the convex distal portion is nested within a concavity of the concave proximal portion, and wherein the convex distal portion is connected to the concave proximal portion at a proximal location on the convex distal portion.

In FIGS. 1 through 6, an intrasaccular aneurysm occlusion device has a convex distal portion with a globular shape (e.g. spherical, oblate spherical, ellipsoidal, or egg shape) and a concave proximal portion with a bowl shape (e.g. hemispherical, half-ellipsoidal, half-toroidal, inverted umbrella, or parabolic shape).

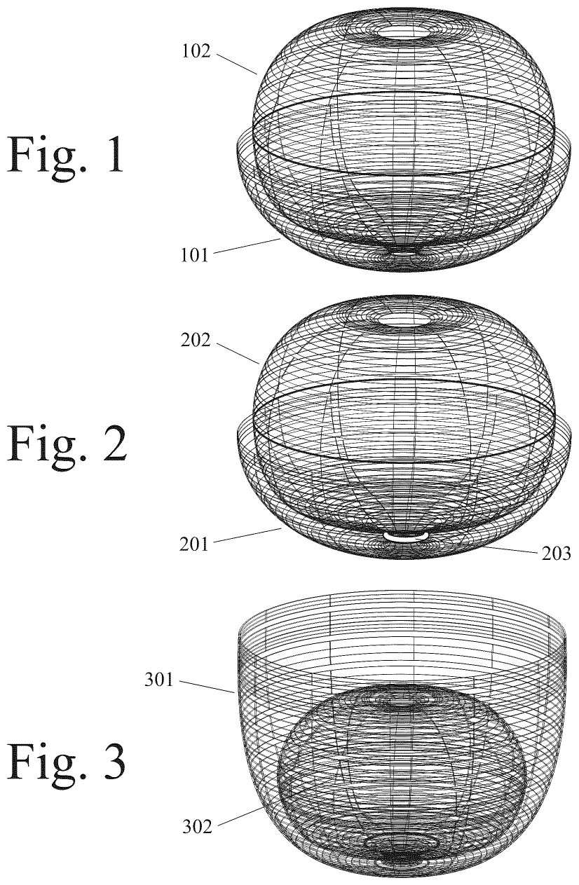

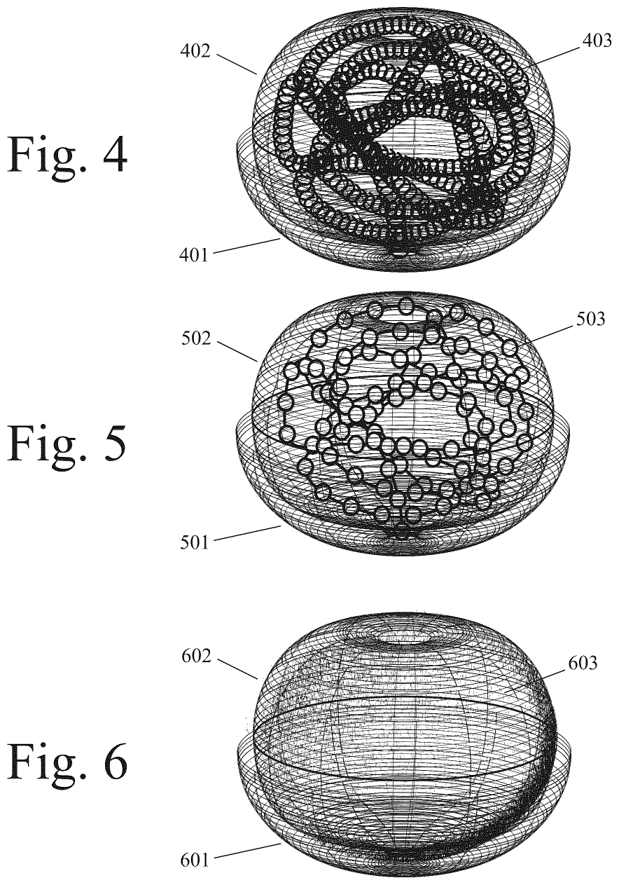

FIG. 1 shows this device without a radial constraint and without embolic members and/or material in the convex distal portion. FIG. 2 shows this device with a radial constraint, but without embolic members and/or material in the convex distal portion. FIG. 3 shows this device wherein the entire convex distal portion is nested within the concavity of the concave proximal portion. FIG. 4 shows this device with embolic coils in the convex distal portion. FIG. 5 shows this device with “string-of-pearls” embolic strands in the convex distal portion. FIG. 6 shows this device with congealing liquid or gelatinous material in the convex distal portion.

With respect to specific components, FIG. 1 shows an example of an intrasaccular aneurysm occlusion device comprising: a convex distal portion 102 which is inserted into an aneurysm sac; and a concave proximal portion 101 which is inserted into the aneurysm sac and expands to cover the aneurysm neck; wherein the convex distal portion is between the concave proximal portion and the dome of the aneurysm sac, wherein at least part of the convex distal portion is nested within a concavity of the concave proximal portion, and wherein the convex distal portion is connected to the concave proximal portion at a proximal location on the convex distal portion.

The example shown in FIG. 1 can also be described as an intrasaccular aneurysm occlusion device comprising: a globular distal portion which is inserted into an aneurysm sac; and a bowl-shaped proximal portion which is inserted into the aneurysm sac and expands to cover the aneurysm neck; wherein the globular distal portion is between the bowl-shaped proximal portion and the dome of the aneurysm sac, wherein between 25% and 75% of the globular distal portion is nested within a concavity of the bowl-shaped proximal portion, and wherein the globular distal portion is connected to the bowl-shaped proximal portion at a proximal location on the globular distal portion.

In FIG. 1, a convex distal portion has a first (proximal-to-distal) height and a concave proximal portion has a second (proximal-to-distal) height, wherein the first height is greater than the second height. In this example, the first height is at least 50% greater than the second height. In this example, between 25% and 75% of a convex distal portion is nested within the concavity of a concave proximal portion. In this example, between 25% and 75% of a convex distal portion overlaps a concave proximal portion. Portion width is defined herein in as a distance in a plane which is orthogonal to the proximal-to-distal axis of a portion. In this example, the concave proximal portion is wider than the convex distal portion.

In this example, the convex distal portion has a globular shape. In various examples, a convex distal portion can have a shape which is selected from the group consisting of: spherical, oblate spherical, ellipsoidal, ovaloid, apple shape, barrel shape, pumpkin shape, cardioid shape, limacon shape, hourglass shape, and egg shape. In an example, a convex distal portion can be more flexible than the concave proximal portion. In an example, the shape of a convex distal portion can change if embolic members and/or material are inserted into it. In an example, the shape of a convex distal portion before insertion of embolic members and/or material can be selected from the group consisting of: spherical, oblate spherical, ellipsoidal, ovaloid, apple shape, barrel shape, pumpkin shape, cardioid shape, limacon shape, hourglass shape, and egg shape. In an example, a convex distal portion can conform to the irregular shape of the walls of an irregularly-shaped aneurysm sac after insertion of embolic members and/or material into the convex distal portion. In an example, the convex distal portion can be a flexible net or mesh.

In this example, the concave proximal portion has a bowl shape. In various examples, a concave proximal portion can have a shape which is selected from the group consisting of: hemispherical, half-ellipsoidal, half-toroidal (e.g. lower half), inverted umbrella shape, inverted dome shape, parabolic, and conic section. In an example, a concave proximal portion can have a radially-compressed configuration as it is delivered through a catheter into an aneurysm sac. In an example, a concave proximal portion can self-expand radially after it has been inserted into the aneurysm sac. In an example, a concave proximal portion can be expanded radially after it has been inserted into the aneurysm sac by moving a wire to which it is attached or by the application of electrical energy.

In this example, the distal end of the convex distal portion has a circular opening. In this example, the distal end of the convex distal portion has a relatively-flat (e.g. planar) circular opening. In another example, a distal end of a convex distal portion can have a three-dimensional funnel or half-hyperboloidal shaped opening which extends into the interior of the convex distal portion. In another example, a distal end of a convex distal portion can have a funnel or half-hyperboloidal shaped opening which extends into the interior of the convex distal portion, spanning between 10% and 30% of the (proximal-to-distal) height of the convex distal portion. In another example, a distal end of a convex distal portion can have a funnel or half-hyperboloidal shaped opening which extends into the interior of the convex distal portion, spanning between 20% and 60% of the (proximal-to-distal) height of the convex distal portion. In another example, a distal end of a convex distal portion can have a funnel or half-hyperboloidal shaped opening which extends into the interior of the convex distal portion, spanning the entire (proximal-to-distal) height of the convex distal portion.

In this example, the distal end of the convex distal portion is not inverted. In another example, a distal end of a convex distal portion can be inverted. In another example, a distal end of a convex distal portion can radially-constrained and inverted. In another example, a distal end of a convex distal portion can radially-constrained by an annular ring, band, or wire and inverted. In another example, a distal end of a convex distal portion can be inverted and protrude into the interior of the convex distal portion. In another example, a distal end of a convex distal portion can be inverted and span between 10% and 30% of the (proximal-to-distal) height of the convex distal portion. In another example, a distal end of a convex distal portion can be inverted and span between 20% and 50% of the (proximal-to-distal) height of the convex distal portion. In another example, a distal end of a convex distal portion can be inverted and span the entire (proximal-to-distal) height of the convex distal portion.

In this example, there is no local concavity at the distal end of the convex distal portion. In another example, there can be a local concavity at a distal end of a convex distal portion. In another example, there can be a local concavity at a distal end of a convex distal portion, wherein the width of the local concavity is between 10% and 30% of the maximum width of the convex distal portion. In another example, there can be a local concavity at a distal end of a convex distal portion, wherein the width of the local concavity is between 20% and 40% of the maximum width of the convex distal portion.

In this example, the distal end of the convex distal portion has an opening. In another example, a distal end of a convex distal portion may not have an opening. In another example, a distal end of a convex distal portion can be radially-constrained (e.g. pinched). In another example, a distal end of a convex distal portion can be radially-constrained (e.g. pinched) closed by an annular ring, band, wire, string, or cylinder. In another example, the distal end of a convex distal portion may be closed (e.g. pinched and/or radially-constrained) by a radial constraint (e.g. an annular ring, band, or wire).

In this example, except for the connection between the concave proximal portion and the convex distal portion, there is a gap between the concave proximal portion and the convex distal portion. In an example, this can be a uniform gap (e.g. a gap with a constant distance). In an example, a gap between a concave proximal portion and a convex distal portion where they are nested together can be a uniform gap (e.g. constant distance). In another example, this gap can decrease with greater distance from the connection between the concave proximal portion and the convex distal portion. In another example, this gap can increase with greater distance from the connection between the concave proximal portion and the convex distal portion. In another example, this gap can first decrease and then increase with greater distance from the connection between the concave proximal portion and the convex distal portion. In another example, this gap can first increase and then decrease with greater distance from the connection between the concave proximal portion and the convex distal portion.

In an example, a distal circumference of a concave proximal portion can fit snuggly around the circumference of a convex distal portion, but there can be a gap between the concave proximal portion and the convex distal portion closer to wherein they are connected at their proximal ends (except for the connection itself). In an example, there can be a first average distance between a concave proximal portion and a convex distal portion in the most proximal quartile of the convex distal portion and a second average distance between the concave proximal portion and the convex distal portion in the proximal-to-middle quartile, wherein the second distance is less than the first distance. In an example, there can be a first average distance between a concave proximal portion and a convex distal portion in the most proximal quartile of the convex distal portion and a second average distance between the concave proximal portion and the convex distal portion in the proximal-to-middle quartile, wherein the second distance is less than half of the first distance.

In this example, the convex distal portion and the concave proximal portion are coaxial as well as being nested. In this example, at least a portion of their central proximal-to-distal axes overlap. In this example, a convex distal portion and a concave proximal portion are connected around their central proximal-to-distal axes. In this example, a convex distal portion and a concave proximal portion are radially-symmetric around their central proximal-to-distal axes. In another example, a concave proximal portion can be radially-symmetric, but a convex distal portion is not. In another example, a convex distal portion can have a first, unexpanded, configuration in which it is radially symmetric and a second, expanded, configuration in which it is not radially symmetric.

In this example, a cross-section (in a plane which is orthogonal to a proximal-to-distal axis) of a convex distal portion has circular perimeter. In an example, a cross-section (in a plane which is orthogonal to a proximal-to-distal axis) of a convex distal portion can have an elliptical or oval perimeter. In another example, a cross-section (in a plane which is orthogonal to a proximal-to-distal axis) of a convex distal portion can have an undulating (e.g. sinusoidal) and/or lobed perimeter.

In this example, the convex distal portion and the concave proximal portion are portions of the same continuous member. In this example, the convex distal portion and the concave proximal portion are made by radially-constraining and/or inverting (or everting) a single continuous member. In this example, the convex distal portion and the concave proximal portion are made by radially-constraining and/or inverting (or everting) a continuous tubular mesh. In an example, a convex distal portion and a concave proximal portion can be formed by radially-constraining and/or inverting (or everting) a tubular wire mesh or braid.

In an example, a convex distal portion and a concave proximal portion can be formed by radially-constraining a tubular mesh (or braid) at one or more points along its longitudinal axis and then inverting (or everting) the tubular mesh (or braid). In an example, a convex distal portion and a concave proximal portion can be formed by radially-constraining a tubular mesh (or braid) at one or both ends and then inverting (or everting) the tubular mesh (or braid). In an example, a convex distal portion and a concave proximal portion can be formed by radially-constraining a tubular mesh (or braid) at two locations along its longitudinal axis. In an example, a convex distal portion and a concave proximal portion can be formed by inverting a tubular mesh (or braid) at two locations along its longitudinal axis.

In an example, a convex distal portion and a concave proximal portion can be formed by radially-constraining a tubular mesh (or braid) at one or both ends, radially constraining the tubular mesh (or braid) at a middle point (between the ends), and then inverting (or everting) the tubular mesh (or braid). In an example, a convex distal portion and a concave proximal portion can be formed by radially-constraining a tubular mesh (or braid) at one or both ends, radially constraining the tubular mesh (or braid) at a middle point (between the ends), and then inverting (or everting) the tubular mesh (or braid) multiple times.

In an example, a convex distal portion and a concave proximal portion can be formed by radially-constraining a tubular mesh (or braid) at multiple points along its longitudinal axis. In an example, a convex distal portion and a concave proximal portion can be formed by inverting a tubular mesh (or braid) at multiple points along its longitudinal axis. In an example, a convex distal portion and a concave proximal portion can be formed by radially-constraining and inverting a tubular mesh (or braid) at multiple points along its longitudinal axis. In an example, a convex distal portion and a concave proximal portion can be formed by radially-constraining a tubular mesh (or braid) at three locations along its longitudinal axis. In an example, a convex distal portion and a concave proximal portion can be formed by inverting a tubular mesh (or braid) at three locations along its longitudinal axis.

In an example, a convex distal portion and a concave proximal portion can be formed by radially-constraining and inverting (or everting) a tubular member (e.g. tubular mesh or braid). In an example, a tubular member can be radially-constrained by a radial constraint which is selected from the group consisting of: annular ring, band, cylinder, or washer; circular wire, strap, filament, or string; helical wire or spring.

In an example, a radial constraint can be a compound component with an outer part and an inner part. In an example, the outer and inner parts can be nested, coaxial, and/or concentric. In an example, a radial-constraint can include an outer part (on the outside of the tubular member) and an inner part (on the inside of the tubular member), wherein the tubular member is held between (e.g. passes between) the outer part and the inner part. In an example, there can be an opening through the inner part through which embolic members and/or material is inserted into the convex distal portion.

In an example, a radial-constraint can include two nested (e.g. coaxial or concentric) bands, rings, or cylinders comprising: an outer band, ring, or cylinder (on the outside of the tubular member) and an inner band, ring, or cylinder (on the inside of the tubular member), wherein the tubular member is held between (e.g. passes through) the outer band, ring, or cylinder and the inner band, ring, or cylinder, and wherein embolic members (or material) are inserted in the convex distal portion through an opening (e.g. lumen) the inner band, ring, or cylinder.

In an example, a convex distal portion and a concave proximal portion can be formed from a tubular member (e.g. wire mesh or braid), wherein the tubular member has longitudinal variation in width, wall thickness, wire thickness, braid or weave density, type or thickness of coating, and material composition. In an example, this tubular mesh can be tapered. In an example, a proximal portion of the tubular mesh can be more dense, stiffer, thicker, and/or less porous than a distal portion of the tubular mesh. In an example, a proximal portion of the tubular mesh can be coated to make it more dense, stiffer, thicker, and/or less porous than a distal portion of the tubular mesh.

In this example, the convex distal portion and the concave proximal portion are made from the same continuous piece of material (e.g. a tubular mesh). In another example, a convex distal portion and a concave proximal portion can be made separately and then connected together. In another example, a convex distal portion and a concave proximal portion can be made from different materials (and/or with different processes) and then connected together. In another example, a convex distal portion and a concave proximal portion can be made from different materials (and/or with different processes) and then connected together before they are inserted into a catheter for deliver into an aneurysm sac. In another example, a convex distal portion and a concave proximal portion can be made from different materials (and/or with different processes) and then connected after they have been inserted into an aneurysm sac.

In an example, a concave proximal portion can be less elastic (e.g. made with less elastic material) than a convex distal portion. In an example, the concave proximal portion can be thicker (e.g. made with thicker wires) than the convex distal portion. In an example, the concave proximal portion can be less compressible (e.g. made with higher durometer material) than the convex distal portion. In an example, the concave proximal portion can be denser (e.g. made with a denser braid or weave) than the convex distal portion. In an example, the concave proximal portion can be less porous (e.g. made with a liquid-impermeable layer) than the convex distal portion. In an example, the concave proximal portion can have more layers (e.g. two or three layers) than the convex distal portion. In an example, the concave proximal portion can comprise a liquid-impermeable (e.g. polymer membrane) layer between two wire layers.

In an example, a convex distal portion can be more elastic (e.g. made with more elastic material) than a concave proximal portion. In an example, the convex distal portion can be thinner (e.g. made with thinner wires) than the concave proximal portion. In an example, the convex distal portion can be more compressible (e.g. made with lower durometer material) than the concave proximal portion. In an example, the convex distal portion can be less dense (e.g. made with a less dense braid or weave) than the concave proximal portion. In an example, the convex distal portion can be more porous (e.g. made with larger holes) than the concave proximal portion. In an example, the convex distal portion can have fewer layers than the concave proximal portion.

In this example, the concave proximal portion has one layer. In another example, a concave proximal portion can have two layers. In another example, the concave proximal portion can have two layers which are formed by radially-constraining a tubular mesh at its proximal end and a middle location, and then inverting (or everting) the area of the tubular mesh between the proximal end and the middle location from a single layer to a double layer. In another example, a concave proximal portion can have three layers, comprising two wire mesh layers and a membrane between the wire mesh layers. In an example, a concave proximal portion can have multiple layers, but a convex distal portion has only one layer. In an example, a concave proximal portion can have more layers than a convex distal portion.

In an example, a convex distal portion and a concave proximal portion can both be made from wire. In an example, a convex distal portion and a concave proximal portion can both be made by braiding or weaving wires. In an example, a convex distal portion and a concave proximal portion can be made by braiding or weaving nitinol wires. In another example, a convex distal portion and a concave proximal portion can be made by 3D printing. In an example, a convex distal portion can be made from a polymer and a concave proximal portion can be made from metal. In an example, a convex distal portion can be a polymer mesh or net and a concave proximal portion can be a braid or weave of metal wires. In an example, a convex distal portion can be a flexible polymer net or mesh and a concave proximal portion can be a wire mesh or stent.

In an example, a convex distal portion and a concave proximal portion can be attached to each other before they are delivered through a catheter into an aneurysm sac. In an example, a convex distal portion and a concave proximal portion can be inserted into an aneurysm sac at substantially the same time. In an example, a convex distal portion and a concave proximal portion can be nested (e.g. overlap) before and after they are inserted into an aneurysm. In another example, a convex distal portion and a concave proximal portion may not be nested (e.g. not overlap) while they are being delivered through a catheter into an aneurysm sac, but are connected to each other within the aneurysm sac so that they become nested (e.g. overlapping) in the aneurysm sac.

In an example, a convex distal portion and a concave proximal portion can be delivered through a catheter sequentially so that they do not overlap while they are being delivered through the catheter. In an example, a convex distal portion and a concave proximal portion can be separated by a longitudinal distance as they are being delivered through a catheter sequentially, but are moved closer to each other (and connected) after they have been inserted into an aneurysm sac.

In an example, a convex distal portion and a concave proximal portion are not nested as they are delivered sequentially through a catheter into an aneurysm sac, but are nested after they are connected to each other within the aneurysm sac. In an example, a convex distal portion and a concave proximal portion do not overlap as they are delivered sequentially through a catheter into an aneurysm sac, but do overlap after they are connected to each other within the aneurysm sac.

In an example, a method for deploying an intrasaccular aneurysm occlusion device can comprise: inserting a convex distal portion of a device into an aneurysm sac; inserting a concave proximal portion of the device into the aneurysm sac; drawing the convex distal portion and the concave proximal portion closer together in the aneurysm sac; and connecting the convex distal portion and the concave proximal portion together in the aneurysm sac.

In an example, a convex distal portion and a concave proximal portion can be separated by a longitudinal distance as they are being delivered through a catheter sequentially, but are moved closer to each other (and connected) by movement of a wire (connected to one or both of them) after they have been inserted into an aneurysm sac. In an example, this device can further comprise a wire which is attached to the convex distal portion. In an example, this device can further comprise a wire which is attached to the distal end of the convex distal portion, wherein the convex distal portion is drawn closer to the concave proximal portion when the wire is pulled.

In an example, this device can further comprise a wire which is attached to the concave proximal portion. In an example, this device can further comprise a wire which is attached to the concave proximal portion, wherein the concave proximal portion is drawn closer to the convex distal portion when the wire is pushed. In an example, this device can further comprise a two wires, one wire which is attached to the convex distal portion and one wire which is attached to the concave proximal portion, wherein the pulling or pushing one or both wires moves the two portions closer together.

In an example, a convex distal portion and a concave proximal portion can be separated by a longitudinal distance as they are being delivered through a catheter sequentially, but are moved closer to each other (and connected) by application of electricity (to one or both of them) after they have been inserted into an aneurysm sac.

With respect to terminology, in various examples a convex distal portion can also be referred to as: a distal ball stent, a distal flexible net, a distal flexible net or mesh, a distal stent, and an intrasaccular arcuate distal stent. With respect to terminology, in various examples a concave proximal portion can also be referred to as: a bowl-shaped mesh, an intrasaccular arcuate proximal stent, a proximal bowl-shaped mesh, a proximal hemispherical stent, a proximal stent, and a resilient wider-than-neck portion.

In an example, a resilient wider-than-neck portion (e.g. concave proximal portion) of a device can have a bowl shape. In an example, a resilient wider-than-neck portion (e.g. concave proximal portion) of a device can have a bowl, hemispherical, inverted dome, paraboloid, and/or inverted umbrella shape. In an example, a bowl-shaped mesh (e.g. concave proximal portion) can be hemispherical.

In an example, an intrasaccular aneurysm occlusion device can comprise: an intrasaccular arcuate distal stent (e.g. convex distal portion); and an intrasaccular arcuate proximal stent (e.g. concave proximal portion); wherein the proximal stent (e.g. concave proximal portion) has a concavity into which a portion of the distal stent (e.g. convex distal portion) fits when the device is deployed within an aneurysm sac. In an example, an intrasaccular aneurysm occlusion device can comprise: a distal ball stent (e.g. convex distal portion); and a proximal hemispherical stent (e.g. concave proximal portion); wherein they overlap when deployed within an aneurysm sac. In an example, a distal surface of a proximal hemispherical stent (e.g. concave proximal portion) can overlap a proximal surface of a distal ball stent (e.g. convex distal portion).

In an example, a valve in an opening in a concave proximal portion of a device can open when an embolic member is pushed through it and close after the embolic member passes through. In another example, a valve can be remotely opened and/or closed by the operator of the device. In an example, a valve can be remotely opened and/or closed by an operator by pulling a filament. In an example, a valve can be remotely opened and/or closed by an operator by pushing, pulling, or rotating a wire. In an example, a valve can be remotely opened and/or closed by an operator by the application of electromagnetic energy. In an example, distal and proximal stents (e.g. concave proximal and convex distal portions) can be connected by a wire and moved toward each other when a user pulls, rotates, or pushes the wire.

In an example, an intrasaccular aneurysm occlusion device can comprise: a proximal bowl-shaped mesh (e.g. concave proximal portion) which is radially-expanded to bridge an aneurysm; a distal flexible net or mesh (e.g. convex distal portion) which is nested within a concavity of the bowl-shaped mesh (e.g. concave proximal portion), wherein the distal flexible net or mesh (e.g. convex distal portion) expands to fill the dome of the aneurysm; and a valve in the proximal bowl-shaped mesh (e.g. concave proximal portion) through which embolic members (e.g. embolic coils, hydrogels, microsponges, beads, or string-of-pearls embolic strands) pass into the distal flexible net (e.g. convex distal portion).

In an example, a string-of-pearls embolic strand can be defined as a series or sequence of embolic members (e.g. beads, polyhedrons, hydrogel pieces, microsponges, or other embolic components) which are connected by flexible longitudinal members (e.g. thin wires, strands, strings, springs, sutures, ribbons, or filaments). In an example, the centers and/or centroids of embolic members (e.g. beads, polyhedrons, hydrogel pieces, microsponges, or other embolic components) in a string-of-pearls embolic strand can be connected by flexible longitudinal members (e.g. thin wires, strands, strings, springs, sutures, ribbons, or filaments). In an example, embolic members (e.g. beads, polyhedrons, hydrogel pieces, microsponges, or other embolic components) in a string-of-pearls embolic strand can be pairwise interconnected by flexible longitudinal members (e.g. thin wires, strands, strings, springs, sutures, ribbons, or filaments).

In an example, an intrasaccular aneurysm occlusion device can comprise: a distal flexible net (e.g. convex distal portion) which is inserted into an aneurysm; a proximal bowl-shaped mesh (e.g. concave proximal portion) which is radially expanded within the aneurysm to bridge the neck of the aneurysm; a central opening in the bowl-shaped mesh (e.g. concave proximal portion); a valve in the central opening; and a string-of-pearls embolic member (e.g. a longitudinal series of embolic components which are connected by a flexible filament or wire) which is delivered through a catheter and inserted through the valve into the distal flexible net (e.g. convex distal portion), thereby expanding the distal flexible net (e.g. convex distal portion) to fill the sac of even an irregularly-shaped aneurysm. Relevant example variations discussed elsewhere in this disclosure or priority-linked disclosures can also be applied to the example shown in FIG. 1.

The example shown in FIG. 2 is like the one in FIG. 1 except that it includes a radial constraint (e.g. an annular ring, band, cylinder, or wire) around the connection between the convex distal portion and the concave proximal portion. FIG. 2 shows an example of an intrasaccular aneurysm occlusion device comprising: a convex distal portion 202 which is inserted into an aneurysm sac; a concave proximal portion 201 which is inserted into the aneurysm sac and expands to cover the aneurysm neck; and an annular constraint 203 which radially constrains the connection between the convex distal portion and the concave proximal portion; wherein the convex distal portion is between the concave proximal portion and the dome of the aneurysm sac, wherein at least part of the convex distal portion is nested within a concavity of the concave proximal portion, and wherein the convex distal portion is connected to the concave proximal portion at a proximal location on the convex distal portion.

More specifically, the example shown in FIG. 2 can be described as an intrasaccular aneurysm occlusion device comprising: a globular distal portion which is inserted into an aneurysm sac; a bowl-shaped proximal portion which is inserted into the aneurysm sac and expands to cover the aneurysm neck; and a radial constraint around the connection between the convex distal portion and the concave proximal portion; wherein the globular distal portion is between the bowl-shaped proximal portion and the dome of the aneurysm sac, wherein between 25% and 75% of the globular distal portion is nested within a concavity of the bowl-shaped proximal portion, and wherein the globular distal portion is connected to the bowl-shaped proximal portion at a proximal location on the globular distal portion. Example variations discussed elsewhere in this disclosure or priority-linked disclosures can also be applied to this example where relevant.

The example shown in FIG. 3 is like the one in FIG. 1 except that the height of the concave distal portion is greater than the height of the concave proximal portion. In other words, the entire convex distal portion is nested within the concave proximal portion. FIG. 3 shows an example of an intrasaccular aneurysm occlusion device comprising: a convex distal portion 302 which is inserted into an aneurysm sac; and a concave proximal portion 301 which is inserted into the aneurysm sac and expands to cover the aneurysm neck; wherein the convex distal portion is between the proximal surface of the concave proximal portion and the dome of the aneurysm sac, wherein the entire convex distal portion is nested within a concavity of the concave proximal portion, and wherein the convex distal portion is connected to the concave proximal portion at a proximal location on the convex distal portion.

More specifically, the example shown in FIG. 3 can be described as an intrasaccular aneurysm occlusion device comprising: a globular distal portion which is inserted into an aneurysm sac; and a bowl-shaped proximal portion which is inserted into the aneurysm sac and expands to cover the aneurysm neck; wherein the globular distal portion is between the proximal surface of the bowl-shaped proximal portion and the dome of the aneurysm sac, wherein the entire globular distal portion is nested within a concavity of the bowl-shaped proximal portion, and wherein the globular distal portion is connected to the bowl-shaped proximal portion at a proximal location on the globular distal portion.

In this example, the convex distal portion has a first (proximal-to-distal) height and the concave proximal portion has a second (proximal-to-distal height), wherein the second height is greater than the first height. In this example, the convex distal portion has a first height and the concave proximal portion has a second height, wherein the second height is at least 50% greater than the first height. In this example, the entire convex distal portion is nested within the concavity of the concave proximal portion. Example variations discussed elsewhere in this disclosure or priority-linked disclosures can also be applied to this example where relevant.

The example shown in FIG. 4 is like the one in FIG. 1 except that embolic members and/or material have been inserted into the convex distal portion. In this example, the embolic members are coils. FIG. 4 shows an example of an intrasaccular aneurysm occlusion device comprising: a convex distal portion 402 which is inserted into an aneurysm sac; a concave proximal portion 401 which is inserted into the aneurysm sac and expands to cover the aneurysm neck; and embolic members and/or material 403 which is inserted into the convex distal portion; wherein the convex distal portion is between the concave proximal portion and the dome of the aneurysm sac, wherein at least part of the convex distal portion is nested within a concavity of the concave proximal portion, and wherein the convex distal portion is connected to the concave proximal portion at a proximal location on the convex distal portion.

More specifically, the example shown in FIG. 4 can be described as an intrasaccular aneurysm occlusion device comprising: a globular distal portion which is inserted into an aneurysm sac; a bowl-shaped proximal portion which is inserted into the aneurysm sac and expands to cover the aneurysm neck; and embolic coils which are inserted into the globular distal portion; wherein the globular distal portion is between the bowl-shaped proximal portion and the dome of the aneurysm sac, wherein between 25% and 75% of the globular distal portion is nested within a concavity of the bowl-shaped proximal portion, and wherein the globular distal portion is connected to the bowl-shaped proximal portion at a proximal location on the globular distal portion.

In an example, an intrasaccular aneurysm occlusion device can comprise: a convex distal portion which is inserted into an aneurysm sac; a concave proximal portion which is inserted into the aneurysm sac and expands to cover the aneurysm neck; and embolic members and/or material; wherein the convex distal portion is between the concave proximal portion and the dome of the aneurysm sac, wherein at least part of the convex distal portion is nested within a concavity of the concave proximal portion, wherein the convex distal portion is connected to the concave proximal portion at a proximal location on the convex distal portion, and wherein the embolic members and/or material are inserted through the concave proximal portion into the convex distal portion.

In an example, an intrasaccular aneurysm occlusion device can comprise: a convex distal portion which is inserted into an aneurysm sac; a concave proximal portion which is inserted into the aneurysm sac and expands to cover the aneurysm neck; and embolic members and/or material; wherein the convex distal portion is between the concave proximal portion and the dome of the aneurysm sac, wherein at least part of the convex distal portion is nested within a concavity of the concave proximal portion, wherein the convex distal portion is connected to the concave proximal portion at a proximal location on the convex distal portion, and wherein the embolic members and/or material are inserted through an opening in concave proximal portion into the convex distal portion.

In another example, an intrasaccular aneurysm occlusion device can comprise: a convex distal portion which is inserted into an aneurysm sac; a concave proximal portion which is inserted into the aneurysm sac and expands to cover the aneurysm neck; and embolic members and/or material; wherein the convex distal portion is between the concave proximal portion and the dome of the aneurysm sac, wherein at least part of the convex distal portion is nested within a concavity of the concave proximal portion, wherein the convex distal portion is connected to the concave proximal portion at a proximal location on the convex distal portion, and wherein the embolic members and/or material are inserted through the convex distal portion into the aneurysm sac.

In this example, embolic members (or material) are inserted into the convex distal portion. In this example, embolic members are coils. In another example, embolic members can be “string of pearl” strands. A “string of pearl” embolic strand can be a series (or sequence) of embolic pieces (e.g. embolic beads, microsponges, or pieces of hydrogel) which are connected by longitudinal strands (e.g. flexible wires, strings, sutures, or springs). One or more “string of pearl” embolic strands can be inserted into the convex distal portion. In another example, embolic members can be separate embolic pieces (e.g. embolic beads, microsponges, or pieces of hydrogel) can be inserted into the convex distal portion. In another example, embolic material can be a liquid or gel which congeals after insertion into an aneurysm sac.

In an example, embolic members (or material) can be inserted through a concave proximal portion into a convex distal portion. In an example, embolic members (or material) can be inserted through an opening in a concave proximal portion into a convex distal portion. In an example, embolic members (or material) can be inserted through an opening between the concave proximal portion and the convex distal portion into the convex distal portion. In an example, embolic members (or material) can be inserted through a concave proximal portion and a convex distal portion into the aneurysm sac (e.g. the aneurysm dome).

In an example, insertion of embolic members (or material) into a convex distal portion can expand a convex distal portion. In an example, insertion of embolic members (or material) into a convex distal portion can expand and change the shape of the convex distal portion. In an example, insertion of embolic members (or material) into a convex distal portion can expand the convex distal portion so that it conforms to the walls of even an irregularly-shaped aneurysm sac. In an example, insertion of embolic members (or material) into a convex distal portion can expand the convex distal portion in a radially-asymmetric manner, enabling it to fill a non-radially-symmetric (e.g. non-spherical) aneurysm sac. In an example, insertion of embolic members (or material) into a convex distal portion can expand the convex distal portion in an irregular manner, enabling it to fill an irregularly shaped (e.g. non-spherical) aneurysm sac. In an example, embolic members (or material) can be inserted into, and contained within, an convex distal portion, thereby expanding the convex distal portion to fill the interior of the aneurysm sac.

In an example, there can be one or more openings in a convex distal portion and a concave proximal portion through which embolic members (or material) are inserted into the convex distal portion. In an example, there can be one or more openings in a convex distal portion and a concave proximal portion along their central longitudinal axes, wherein embolic members (or material) are inserted through these one or more openings into the convex distal portion. In an example, there can be a first opening in the convex distal portion and a second opening in the concave proximal portion, wherein these first and second openings are aligned to enable insertion of embolic members (or material) through the openings into the convex distal portion. In an example, there can be an opening in a radial constraint which radially-constrains a tubular mesh from which the convex distal portion and the concave proximal portion are formed, wherein embolic members (or material) are inserted through this opening into the convex distal portion.

In an example, there can be a valve in an opening through a concave proximal portion. In an example, this valve can allow embolic members and/or material to enter a convex distal portion when the valve is open and can prevent embolic members and/or material from escaping out of the convex distal portion when the valve is closed. In an example, this valve can operate passively. In an example, a valve can be a passive one-way valve which allows embolic members and/or material to enter, but not exit, a convex distal portion or a robotic system. In another example, this valve can be actively opened or closed (remotely) by an operator who is deploying the device. In an example, a valve can be actively opened or closed by a mechanism selected from the group consisting of: application of electrical energy to the valve; pulling or pushing a wire connected to the valve; rotating a wire connected to the valve; pulling a filament or string connected to the valve; activating a small-scale electrical actuator; activating a hydraulic mechanism; activating a pneumatic mechanism; and rotating a catheter.

In an example, a method for deploying an intrasaccular aneurysm occlusion device can comprise: forming a convex distal portion of a device and a concave proximal portion of the device; inserting the convex distal portion and the concave proximal portion into an into the aneurysm sac; and inserting embolic members and/or material into the convex distal portion. In an example, a method for deploying an intrasaccular aneurysm occlusion device can comprise: forming a convex distal portion of a device and a concave proximal portion of the device by radially-constraining and inverting a tubular mesh; inserting the convex distal portion and the concave proximal portion into an into the aneurysm sac; and inserting embolic members and/or material into the convex distal portion.

In an example, a method for deploying an intrasaccular aneurysm occlusion device can comprise: inserting a convex distal portion of a device into an aneurysm sac; inserting a concave proximal portion of the device into the aneurysm sac; drawing the convex distal portion and the concave proximal portion closer together in the aneurysm sac; connecting the convex distal portion and the concave proximal portion together in the aneurysm sac; and inserting embolic members and/or material into the convex distal portion. Example variations discussed elsewhere in this disclosure or priority-linked disclosures can also be applied to this example where relevant.

The example shown in FIG. 5 is like the one in FIG. 4 except that the embolic members are “string-of-pearls” embolic strands instead of coils. FIG. 5 shows an example of an intrasaccular aneurysm occlusion device comprising: a convex distal portion 502 which is inserted into an aneurysm sac; a concave proximal portion 501 which is inserted into the aneurysm sac and expands to cover the aneurysm neck; and embolic members and/or material 503 which is inserted into the convex distal portion; wherein the convex distal portion is between the concave proximal portion and the dome of the aneurysm sac, wherein at least part of the convex distal portion is nested within a concavity of the concave proximal portion, and wherein the convex distal portion is connected to the concave proximal portion at a proximal location on the convex distal portion.

More specifically, the example shown in FIG. 5 can be described as an intrasaccular aneurysm occlusion device comprising: a globular distal portion which is inserted into an aneurysm sac; a bowl-shaped proximal portion which is inserted into the aneurysm sac and expands to cover the aneurysm neck; and “string-of-pearls” embolic strands which are inserted into the globular distal portion; wherein the globular distal portion is between the bowl-shaped proximal portion and the dome of the aneurysm sac, wherein between 25% and 75% of the globular distal portion is nested within a concavity of the bowl-shaped proximal portion, and wherein the globular distal portion is connected to the bowl-shaped proximal portion at a proximal location on the globular distal portion.

In an example, a string-of-pearls embolic strand can be defined as a series or sequence of embolic members (e.g. beads, polyhedrons, hydrogel pieces, microsponges, or other embolic components) which are connected by flexible longitudinal members (e.g. thin wires, strands, strings, springs, sutures, ribbons, or filaments). In an example, the centers and/or centroids of embolic members (e.g. beads, polyhedrons, hydrogel pieces, microsponges, or other embolic components) in a string-of-pearls embolic strand can be connected by flexible longitudinal members (e.g. thin wires, strands, strings, springs, sutures, ribbons, or filaments). In an example, embolic members (e.g. beads, polyhedrons, hydrogel pieces, microsponges, or other embolic components) in a string-of-pearls embolic strand can be pairwise interconnected by flexible longitudinal members (e.g. thin wires, strands, strings, springs, sutures, ribbons, or filaments).

In an example, there can be variation in the size of embolic pieces along the length of a “string-of-pearls” embolic strand. In an example, embolic pieces which are closer to the distal end of a “string-of-pearls” embolic strand can be larger than embolic pieces which are father from this distal end. In an example, there can be variation in the compressibility and/or durometer of embolic pieces along the length of a “string-of-pearls” embolic strand. In an example, embolic pieces which are closer to the distal end of a “string-of-pearls” embolic strand can be less compressible and/or higher durometer than embolic pieces which are father from this distal end. In an example, there can be variation in the distance between embolic pieces along the length of a “string-of-pearls” embolic strand. In an example, embolic pieces which are closer to the distal end of a “string-of-pearls” embolic strand can be closer together than embolic pieces which are father from this distal end. Example variations discussed elsewhere in this disclosure or priority-linked disclosures can also be applied to this example where relevant.

The example shown in FIG. 6 is like the one in FIG. 4 except that embolic material is liquid or gelatinous material (e.g. a “liquid embolic”) which congeals in the aneurysm sac. FIG. 6 shows an example of an intrasaccular aneurysm occlusion device comprising: a convex distal portion 602 which is inserted into an aneurysm sac; a concave proximal portion 601 which is inserted into the aneurysm sac and expands to cover the aneurysm neck; and embolic members and/or material 603 which is inserted into the convex distal portion; wherein the convex distal portion is between the concave proximal portion and the dome of the aneurysm sac, wherein at least part of the convex distal portion is nested within a concavity of the concave proximal portion, and wherein the convex distal portion is connected to the concave proximal portion at a proximal location on the convex distal portion.

More specifically, the example shown in FIG. 6 can be described as an intrasaccular aneurysm occlusion device comprising: a globular distal portion which is inserted into an aneurysm sac; a bowl-shaped proximal portion which is inserted into the aneurysm sac and expands to cover the aneurysm neck; and congealing liquid or gelatinous embolic material which is inserted into the globular distal portion; wherein the globular distal portion is between the bowl-shaped proximal portion and the dome of the aneurysm sac, wherein between 25% and 75% of the globular distal portion is nested within a concavity of the bowl-shaped proximal portion, and wherein the globular distal portion is connected to the bowl-shaped proximal portion at a proximal location on the globular distal portion. Example variations discussed elsewhere in this disclosure or priority-linked disclosures can also be applied to this example where relevant.

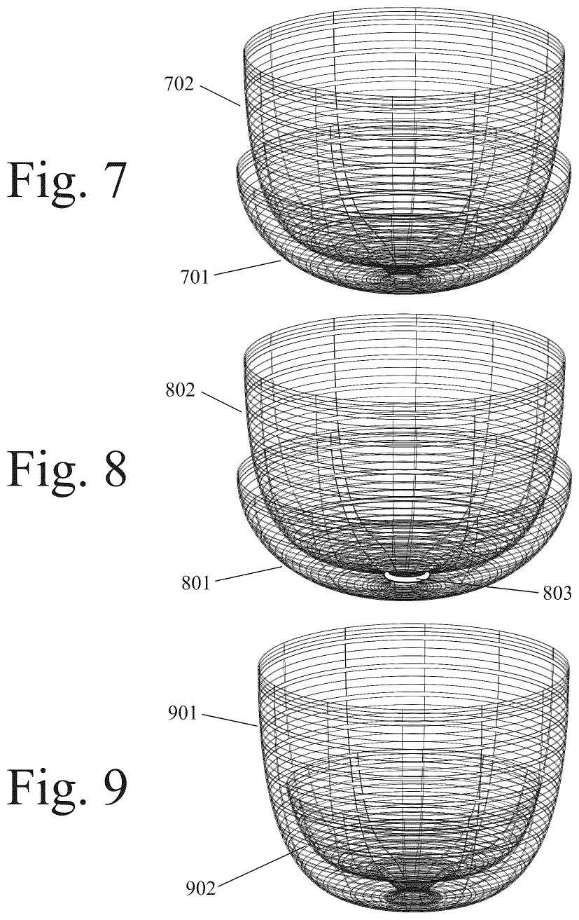

FIGS. 7 through 12 show an example of an intrasaccular aneurysm occlusion device which is similar to the one shown in FIGS. 1 through 6 except that the distal portion is concave (e.g. bowl-shaped) rather than convex (e.g. globular). In FIGS. 7 through 12, both a concave distal portion and a concave proximal portion have bowl shapes (e.g. hemispherical, half-ellipsoidal, half-toroidal, inverted umbrella, or parabolic shapes).

FIGS. 7 through 12 show an example of an intrasaccular aneurysm occlusion device comprising: a concave distal portion which is inserted into an aneurysm sac; and a concave proximal portion which is inserted into the aneurysm sac and expands to cover the aneurysm neck; wherein the concave distal portion is between the concave proximal portion and the dome of the aneurysm sac, wherein at least part of the concave distal portion is nested within a concavity of the concave proximal portion, and wherein the concave distal portion is connected to the concave proximal portion at a proximal location on the concave distal portion.

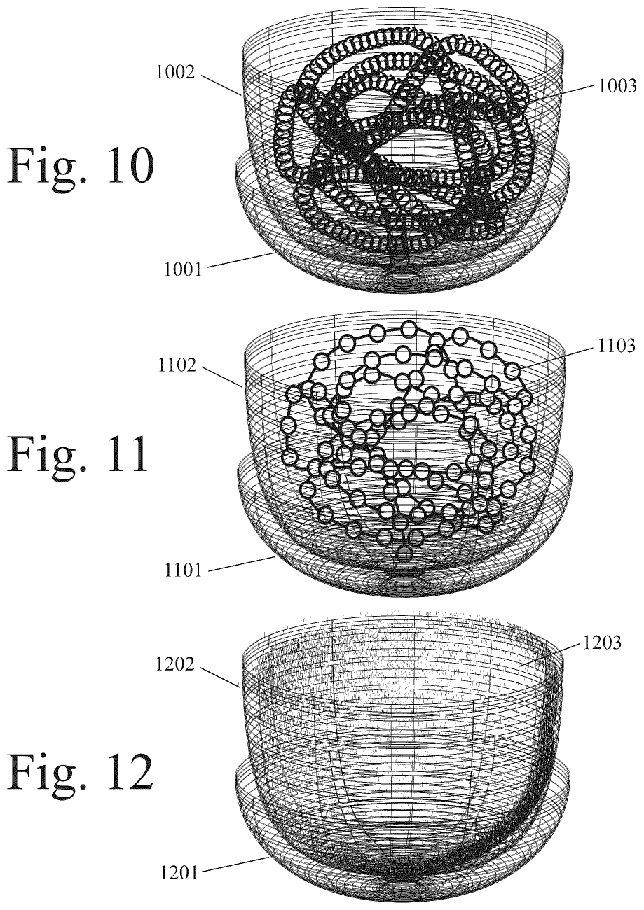

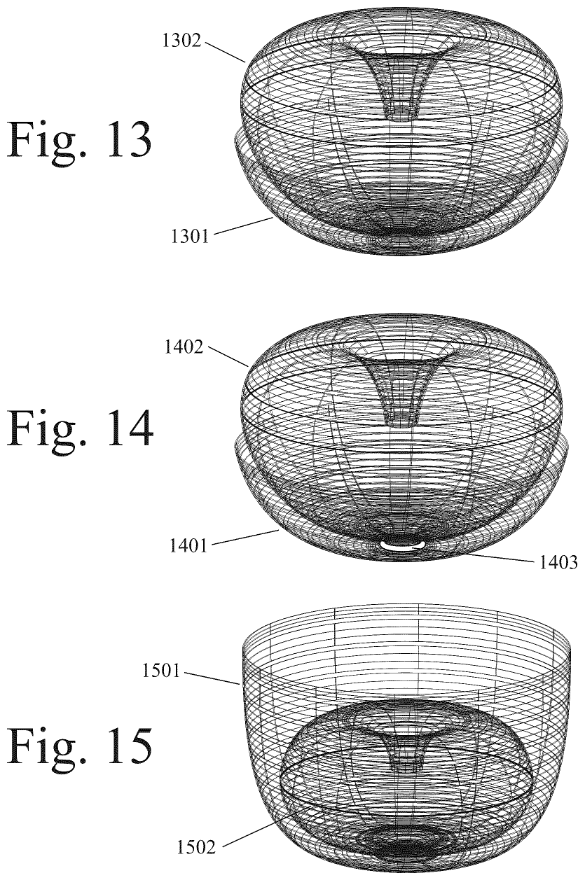

FIG. 7 shows this device without a radial constraint and without embolic members and/or material in the concave distal portion. FIG. 8 shows this device with a radial constraint, but without embolic members and/or material in the concave distal portion. FIG. 9 shows this device wherein the entire concave distal portion is nested within the concavity of the concave proximal portion. FIG. 10 shows this device with embolic coils in the concave distal portion. FIG. 11 shows this device with “string-of-pearls” embolic strands in the concave distal portion. FIG. 12 shows this device with congealing liquid or gelatinous material in the concave distal portion.

With respect to specific components, FIG. 7 shows an example of an intrasaccular aneurysm occlusion device comprising: a concave distal portion 702 which is inserted into an aneurysm sac; and a concave proximal portion 701 which is inserted into the aneurysm sac and expands to cover the aneurysm neck; wherein the concave distal portion is between the concave proximal portion and the dome of the aneurysm sac, wherein at least part of the concave distal portion is nested within a concavity of the concave proximal portion, and wherein the concave distal portion is connected to the concave proximal portion at a proximal location on the concave distal portion.

More specifically, the example shown in FIG. 7 can be described as an intrasaccular aneurysm occlusion device comprising: a bowl-shaped distal portion which is inserted into an aneurysm sac; and a bowl-shaped proximal portion which is inserted into the aneurysm sac and expands to cover the aneurysm neck; wherein the bowl-shaped distal portion is between the bowl-shaped proximal portion and the dome of the aneurysm sac, wherein between 25% and 75% of the bowl-shaped distal portion is nested within a concavity of the bowl-shaped proximal portion, and wherein the bowl-shaped distal portion is connected to the bowl-shaped proximal portion at a proximal location on the bowl-shaped distal portion.

In FIG. 7, a concave distal portion has a first (proximal-to-distal) height and a concave proximal portion has a second (proximal-to-distal) height, wherein the first height is greater than the second height. In this example, the first height is at least 50% greater than the second height. In this example, between 25% and 75% of a concave distal portion is nested within the concavity of a concave proximal portion. In this example, between 25% and 75% of a concave distal portion overlaps a concave proximal portion. Portion width is defined herein in as a distance in a plane which is orthogonal to the proximal-to-distal axis of a portion. In this example, the concave proximal portion is wider than the concave distal portion.

In this example, both the concave distal portion and the concave proximal portion have a bowl shapes. In various examples, the concave distal portion and the concave proximal portion can have shapes which are selected from the group consisting of: hemispherical, half-ellipsoidal, half-toroidal (e.g. lower half), inverted umbrella shape, inverted dome shape, parabolic, carlavian curve shape, and conic section. In an example, a concave distal portion can be more flexible than the concave proximal portion. In an example, the shape of a concave distal portion can change if embolic members and/or material are inserted into it. In an example, a concave distal portion can conform to the irregular shape of the walls of an irregularly-shaped aneurysm sac after insertion of embolic members and/or material into the concave distal portion. In an example, the concave distal portion can be a flexible net or mesh.

In an example, a concave proximal portion can have a radially-compressed configuration as it is delivered through a catheter into an aneurysm sac. In an example, a concave proximal portion can self-expand radially after it has been inserted into the aneurysm sac. In an example, a concave proximal portion can be expanded radially after it has been inserted into the aneurysm sac by moving a wire to which it is attached or by the application of electrical energy.

In this example, except for the connection between the concave proximal portion and the concave distal portion, there is a gap between the concave proximal portion and the concave distal portion. In an example, this can be a uniform gap (e.g. a gap with a constant distance). In an example, a gap between a concave proximal portion and a concave distal portion where they are nested together can be a uniform gap (e.g. constant distance). In another example, this gap can decrease with greater distance from the connection between the concave proximal portion and the concave distal portion. In another example, this gap can increase with greater distance from the connection between the concave proximal portion and the concave distal portion. In another example, this gap can first decrease and then increase with greater distance from the connection between the concave proximal portion and the concave distal portion. In another example, this gap can first increase and then decrease with greater distance from the connection between the concave proximal portion and the concave distal portion.

In an example, a distal circumference of a concave proximal portion can fit snuggly around the circumference of a concave distal portion, but there can be a gap between the concave proximal portion and the concave distal portion closer to wherein they are connected at their proximal ends (except for the connection itself). In an example, there can be a first average distance between a concave proximal portion and a concave distal portion in the most proximal quartile of the concave distal portion and a second average distance between the concave proximal portion and the concave distal portion in the proximal-to-middle quartile, wherein the second distance is less than the first distance. In an example, there can be a first average distance between a concave proximal portion and a concave distal portion in the most proximal quartile of the concave distal portion and a second average distance between the concave proximal portion and the concave distal portion in the proximal-to-middle quartile, wherein the second distance is less than half of the first distance.

In this example, the concave distal portion and the concave proximal portion are coaxial as well as being nested. In this example, at least a portion of their central proximal-to-distal axes overlap. In this example, a concave distal portion and a concave proximal portion are connected around their central proximal-to-distal axes. In this example, a concave distal portion and a concave proximal portion are radially-symmetric around their central proximal-to-distal axes. In another example, a concave proximal portion can be radially-symmetric, but a concave distal portion is not. In another example, a concave distal portion can have a first, unexpanded, configuration in which it is radially symmetric and a second, expanded, configuration in which it is not radially symmetric.

In this example, a cross-section (in a plane which is orthogonal to a proximal-to-distal axis) of a concave distal portion has circular perimeter. In an example, a cross-section (in a plane which is orthogonal to a proximal-to-distal axis) of a concave distal portion can have an elliptical or oval perimeter. In another example, a cross-section (in a plane which is orthogonal to a proximal-to-distal axis) of a concave distal portion can have an undulating (e.g. sinusoidal) and/or lobed perimeter.

In this example, the concave distal portion and the concave proximal portion are portions of the same continuous member. In this example, the concave distal portion and the concave proximal portion are made by radially-constraining and/or inverting (or everting) a single continuous member. In this example, the concave distal portion and the concave proximal portion are made by radially-constraining and/or inverting (or everting) a continuous tubular mesh. In an example, a concave distal portion and a concave proximal portion can be formed by radially-constraining and/or inverting (or everting) a tubular wire mesh or braid.

In an example, a concave distal portion and a concave proximal portion can be formed by radially-constraining a tubular mesh (or braid) at one or more points along its longitudinal axis and then inverting (or everting) the tubular mesh (or braid). In an example, a concave distal portion and a concave proximal portion can be formed by radially-constraining a tubular mesh (or braid) at one or both ends and then inverting (or everting) the tubular mesh (or braid). In an example, a concave distal portion and a concave proximal portion can be formed by radially-constraining a tubular mesh (or braid) at two locations along its longitudinal axis. In an example, a concave distal portion and a concave proximal portion can be formed by inverting a tubular mesh (or braid) at two locations along its longitudinal axis.

In an example, a concave distal portion and a concave proximal portion can be formed by radially-constraining a tubular mesh (or braid) at one or both ends, radially constraining the tubular mesh (or braid) at a middle point (between the ends), and then inverting (or everting) the tubular mesh (or braid). In an example, a concave distal portion and a concave proximal portion can be formed by radially-constraining a tubular mesh (or braid) at one or both ends, radially constraining the tubular mesh (or braid) at a middle point (between the ends), and then inverting (or everting) the tubular mesh (or braid) multiple times.

In an example, a concave distal portion and a concave proximal portion can be formed by radially-constraining a tubular mesh (or braid) at multiple points along its longitudinal axis. In an example, a concave distal portion and a concave proximal portion can be formed by inverting a tubular mesh (or braid) at multiple points along its longitudinal axis. In an example, a concave distal portion and a concave proximal portion can be formed by radially-constraining and inverting a tubular mesh (or braid) at multiple points along its longitudinal axis. In an example, a concave distal portion and a concave proximal portion can be formed by radially-constraining a tubular mesh (or braid) at three locations along its longitudinal axis. In an example, a concave distal portion and a concave proximal portion can be formed by inverting a tubular mesh (or braid) at three locations along its longitudinal axis.

In an example, a concave distal portion and a concave proximal portion can be formed by radially-constraining and inverting (or everting) a tubular member (e.g. tubular mesh or braid). In an example, a tubular member can be radially-constrained by a radial constraint which is selected from the group consisting of: annular ring, band, cylinder, or washer; circular wire, strap, filament, or string; helical wire or spring.

In an example, a radial constraint can be a compound component with an outer part and an inner part. In an example, the outer and inner parts can be nested, coaxial, and/or concentric. In an example, a radial-constraint can include an outer part (on the outside of the tubular member) and an inner part (on the inside of the tubular member), wherein the tubular member is held between (e.g. passes between) the outer part and the inner part. In an example, there can be an opening through the inner part through which embolic members and/or material is inserted into the concave distal portion.

In an example, a radial-constraint can include two nested (e.g. coaxial or concentric) bands, rings, or cylinders comprising: an outer band, ring, or cylinder (on the outside of the tubular member) and an inner band, ring, or cylinder (on the inside of the tubular member), wherein the tubular member is held between (e.g. passes through) the outer band, ring, or cylinder and the inner band, ring, or cylinder, and wherein embolic members (or material) are inserted in the concave distal portion through an opening (e.g. lumen) the inner band, ring, or cylinder.

In an example, a concave distal portion and a concave proximal portion can be formed from a tubular member (e.g. wire mesh or braid), wherein the tubular member has longitudinal variation in width, wall thickness, wire thickness, braid or weave density, type or thickness of coating, and material composition. In an example, this tubular mesh can be tapered. In an example, a proximal portion of the tubular mesh can be more dense, stiffer, thicker, and/or less porous than a distal portion of the tubular mesh. In an example, a proximal portion of the tubular mesh can be coated to make it more dense, stiffer, thicker, and/or less porous than a distal portion of the tubular mesh.

In this example, the concave distal portion and the concave proximal portion are made from the same continuous piece of material (e.g. a tubular mesh). In another example, a concave distal portion and a concave proximal portion can be made separately and then connected together. In another example, a concave distal portion and a concave proximal portion can be made from different materials (and/or with different processes) and then connected together. In another example, a concave distal portion and a concave proximal portion can be made from different materials (and/or with different processes) and then connected together before they are inserted into a catheter for deliver into an aneurysm sac. In another example, a concave distal portion and a concave proximal portion can be made from different materials (and/or with different processes) and then connected after they have been inserted into an aneurysm sac.

In an example, a concave proximal portion can be less elastic (e.g. made with less elastic material) than a concave distal portion. In an example, the concave proximal portion can be thicker (e.g. made with thicker wires) than the concave distal portion. In an example, the concave proximal portion can be less compressible (e.g. made with higher durometer material) than the concave distal portion. In an example, the concave proximal portion can be denser (e.g. made with a denser braid or weave) than the concave distal portion. In an example, the concave proximal portion can be less porous (e.g. made with a liquid-impermeable layer) than the concave distal portion. In an example, the concave proximal portion can have more layers (e.g. two or three layers) than the concave distal portion. In an example, the concave proximal portion can comprise a liquid-impermeable (e.g. polymer membrane) layer between two wire layers.

In an example, a concave distal portion can be more elastic (e.g. made with more elastic material) than a concave proximal portion. In an example, the concave distal portion can be thinner (e.g. made with thinner wires) than the concave proximal portion. In an example, the concave distal portion can be more compressible (e.g. made with lower durometer material) than the concave proximal portion. In an example, the concave distal portion can be less dense (e.g. made with a less dense braid or weave) than the concave proximal portion. In an example, the concave distal portion can be more porous (e.g. made with larger holes) than the concave proximal portion. In an example, the concave distal portion can have fewer layers than the concave proximal portion.

In this example, the concave proximal portion has one layer. In another example, a concave proximal portion can have two layers. In another example, the concave proximal portion can have two layers which are formed by radially-constraining a tubular mesh at its proximal end and a middle location, and then inverting (or everting) the area of the tubular mesh between the proximal end and the middle location from a single layer to a double layer. In another example, a concave proximal portion can have three layers, comprising two wire mesh layers and a membrane between the wire mesh layers. In an example, a concave proximal portion can have multiple layers, but a concave distal portion has only one layer. In an example, a concave proximal portion can have more layers than a concave distal portion.

In an example, a concave distal portion and a concave proximal portion can both be made from wire. In an example, a concave distal portion and a concave proximal portion can both be made by braiding or weaving wires. In an example, a concave distal portion and a concave proximal portion can be made by braiding or weaving nitinol wires. In another example, a concave distal portion and a concave proximal portion can be made by 3D printing. In an example, a concave distal portion can be made from a polymer and a concave proximal portion can be made from metal. In an example, a concave distal portion can be a polymer mesh or net and a concave proximal portion can be a braid or weave of metal wires. In an example, a concave distal portion can be a flexible polymer net or mesh and a concave proximal portion can be a wire mesh or stent.

In an example, a concave distal portion and a concave proximal portion can be attached to each other before they are delivered through a catheter into an aneurysm sac. In an example, a concave distal portion and a concave proximal portion can be inserted into an aneurysm sac at substantially the same time. In an example, a concave distal portion and a concave proximal portion can be nested (e.g. overlap) before and after they are inserted into an aneurysm. In another example, a concave distal portion and a concave proximal portion may not be nested (e.g. not overlap) while they are being delivered through a catheter into an aneurysm sac, but are connected to each other within the aneurysm sac so that they become nested (e.g. overlapping) in the aneurysm sac.

In an example, a concave distal portion and a concave proximal portion can be delivered through a catheter sequentially so that they do not overlap while they are being delivered through the catheter. In an example, a concave distal portion and a concave proximal portion can be separated by a longitudinal distance as they are being delivered through a catheter sequentially, but are moved closer to each other (and connected) after they have been inserted into an aneurysm sac.

In an example, a concave distal portion and a concave proximal portion are not nested as they are delivered sequentially through a catheter into an aneurysm sac, but are nested after they are connected to each other within the aneurysm sac. In an example, a concave distal portion and a concave proximal portion do not overlap as they are delivered sequentially through a catheter into an aneurysm sac, but do overlap after they are connected to each other within the aneurysm sac.

In an example, a method for deploying an intrasaccular aneurysm occlusion device can comprise: inserting a concave distal portion of a device into an aneurysm sac; inserting a concave proximal portion of the device into the aneurysm sac; drawing the concave distal portion and the concave proximal portion closer together in the aneurysm sac; and connecting the concave distal portion and the concave proximal portion together in the aneurysm sac.

In an example, a concave distal portion and a concave proximal portion can be separated by a longitudinal distance as they are being delivered through a catheter sequentially, but are moved closer to each other (and connected) by movement of a wire (connected to one or both of them) after they have been inserted into an aneurysm sac. In an example, this device can further comprise a wire which is attached to the concave distal portion. In an example, this device can further comprise a wire which is attached to the distal end of the concave distal portion, wherein the concave distal portion is drawn closer to the concave proximal portion when the wire is pulled.

In an example, this device can further comprise a wire which is attached to the concave proximal portion. In an example, this device can further comprise a wire which is attached to the concave proximal portion, wherein the concave proximal portion is drawn closer to the concave distal portion when the wire is pushed. In an example, this device can further comprise a two wires, one wire which is attached to the concave distal portion and one wire which is attached to the concave proximal portion, wherein the pulling or pushing one or both wires moves the two portions closer together.

In an example, a concave distal portion and a concave proximal portion can be separated by a longitudinal distance as they are being delivered through a catheter sequentially, but are moved closer to each other (and connected) by application of electricity (to one or both of them) after they have been inserted into an aneurysm sac. Relevant example variations discussed elsewhere in this disclosure or priority-linked disclosures can also be applied to the example shown in FIG. 7.

The example shown in FIG. 8 is like the one in FIG. 7 except that it includes a radial constraint (e.g. an annular ring, band, cylinder, or wire) around the connection between the concave distal portion and the concave proximal portion. FIG. 8 shows an example of an intrasaccular aneurysm occlusion device comprising: a concave distal portion 802 which is inserted into an aneurysm sac; a concave proximal portion 801 which is inserted into the aneurysm sac and expands to cover the aneurysm neck; and an annular constraint 803 which radially constrains the connection between the concave distal portion and the concave proximal portion; wherein the concave distal portion is between the concave proximal portion and the dome of the aneurysm sac, wherein at least part of the concave distal portion is nested within a concavity of the concave proximal portion, and wherein the concave distal portion is connected to the concave proximal portion at a proximal location on the concave distal portion.

More specifically, the example shown in FIG. 8 can be described as an intrasaccular aneurysm occlusion device comprising: a bowl-shaped distal portion which is inserted into an aneurysm sac; a bowl-shaped proximal portion which is inserted into the aneurysm sac and expands to cover the aneurysm neck; and a radial constraint around the connection between the concave distal portion and the concave proximal portion; wherein the bowl-shaped distal portion is between the bowl-shaped proximal portion and the dome of the aneurysm sac, wherein between 25% and 75% of the bowl-shaped distal portion is nested within a concavity of the bowl-shaped proximal portion, and wherein the bowl-shaped distal portion is connected to the bowl-shaped proximal portion at a proximal location on the bowl-shaped distal portion. Example variations discussed elsewhere in this disclosure or priority-linked disclosures can also be applied to this example where relevant.

The example shown in FIG. 9 is like the one in FIG. 7 except that the height of the concave distal portion is greater than the height of the concave proximal portion. In other words, the entire concave distal portion is nested within the concave proximal portion. FIG. 9 shows an example of an intrasaccular aneurysm occlusion device comprising: a concave distal portion 902 which is inserted into an aneurysm sac; and a concave proximal portion 901 which is inserted into the aneurysm sac and expands to cover the aneurysm neck; wherein the concave distal portion is between the proximal surface of the concave proximal portion and the dome of the aneurysm sac, wherein the entire concave distal portion is nested within a concavity of the concave proximal portion, and wherein the concave distal portion is connected to the concave proximal portion at a proximal location on the concave distal portion.

More specifically, the example shown in FIG. 9 can be described as an intrasaccular aneurysm occlusion device comprising: a bowl-shaped distal portion which is inserted into an aneurysm sac; and a bowl-shaped proximal portion which is inserted into the aneurysm sac and expands to cover the aneurysm neck; wherein the bowl-shaped distal portion is between the proximal surface of the bowl-shaped proximal portion and the dome of the aneurysm sac, wherein the entire bowl-shaped distal portion is nested within a concavity of the bowl-shaped proximal portion, and wherein the bowl-shaped distal portion is connected to the bowl-shaped proximal portion at a proximal location on the bowl-shaped distal portion.

In this example, the concave distal portion has a first (proximal-to-distal) height and the concave proximal portion has a second (proximal-to-distal height), wherein the second height is greater than the first height. In this example, the concave distal portion has a first height and the concave proximal portion has a second height, wherein the second height is at least 50% greater than the first height. In this example, the entire concave distal portion is nested within the concavity of the concave proximal portion. Example variations discussed elsewhere in this disclosure or priority-linked disclosures can also be applied to this example where relevant.

The example shown in FIG. 10 is like the one in FIG. 7 except that embolic members and/or material have been inserted into the concave distal portion. In this example, the embolic members are coils. FIG. 10 shows an example of an intrasaccular aneurysm occlusion device comprising: a concave distal portion 1002 which is inserted into an aneurysm sac; a concave proximal portion 1001 which is inserted into the aneurysm sac and expands to cover the aneurysm neck; and embolic members and/or material 1003 which is inserted into the concave distal portion; wherein the concave distal portion is between the concave proximal portion and the dome of the aneurysm sac, wherein at least part of the concave distal portion is nested within a concavity of the concave proximal portion, and wherein the concave distal portion is connected to the concave proximal portion at a proximal location on the concave distal portion.

More specifically, the example shown in FIG. 10 can be described as an intrasaccular aneurysm occlusion device comprising: a bowl-shaped distal portion which is inserted into an aneurysm sac; a bowl-shaped proximal portion which is inserted into the aneurysm sac and expands to cover the aneurysm neck; and embolic coils which are inserted into the bowl-shaped distal portion; wherein the bowl-shaped distal portion is between the bowl-shaped proximal portion and the dome of the aneurysm sac, wherein between 25% and 75% of the bowl-shaped distal portion is nested within a concavity of the bowl-shaped proximal portion, and wherein the bowl-shaped distal portion is connected to the bowl-shaped proximal portion at a proximal location on the bowl-shaped distal portion.

In an example, an intrasaccular aneurysm occlusion device can comprise: a concave distal portion which is inserted into an aneurysm sac; a concave proximal portion which is inserted into the aneurysm sac and expands to cover the aneurysm neck; and embolic members and/or material; wherein the concave distal portion is between the concave proximal portion and the dome of the aneurysm sac, wherein at least part of the concave distal portion is nested within a concavity of the concave proximal portion, wherein the concave distal portion is connected to the concave proximal portion at a proximal location on the concave distal portion, and wherein the embolic members and/or material are inserted through the concave proximal portion into the concave distal portion.

In an example, an intrasaccular aneurysm occlusion device can comprise: a concave distal portion which is inserted into an aneurysm sac; a concave proximal portion which is inserted into the aneurysm sac and expands to cover the aneurysm neck; and embolic members and/or material; wherein the concave distal portion is between the concave proximal portion and the dome of the aneurysm sac, wherein at least part of the concave distal portion is nested within a concavity of the concave proximal portion, wherein the concave distal portion is connected to the concave proximal portion at a proximal location on the concave distal portion, and wherein the embolic members and/or material are inserted through an opening in concave proximal portion into the concave distal portion.

In another example, an intrasaccular aneurysm occlusion device can comprise: a concave distal portion which is inserted into an aneurysm sac; a concave proximal portion which is inserted into the aneurysm sac and expands to cover the aneurysm neck; and embolic members and/or material; wherein the concave distal portion is between the concave proximal portion and the dome of the aneurysm sac, wherein at least part of the concave distal portion is nested within a concavity of the concave proximal portion, wherein the concave distal portion is connected to the concave proximal portion at a proximal location on the concave distal portion, and wherein the embolic members and/or material are inserted through the concave distal portion into the aneurysm sac.

In this example, embolic members (or material) are inserted into the concave distal portion. In this example, embolic members are coils. In another example, embolic members can be “string of pearl” strands. A “string of pearl” embolic strand can be a series (or sequence) of embolic pieces (e.g. embolic beads, microsponges, or pieces of hydrogel) which are connected by longitudinal strands (e.g. flexible wires, strings, sutures, or springs). One or more “string of pearl” embolic strands can be inserted into the concave distal portion. In another example, embolic members can be separate embolic pieces (e.g. embolic beads, microsponges, or pieces of hydrogel) can be inserted into the concave distal portion. In another example, embolic material can be a liquid or gel which congeals after insertion into an aneurysm sac.

In an example, embolic members (or material) can be inserted through a concave proximal portion into a concave distal portion. In an example, embolic members (or material) can be inserted through an opening in a concave proximal portion into a concave distal portion. In an example, embolic members (or material) can be inserted through an opening between the concave proximal portion and the concave distal portion into the concave distal portion. In an example, embolic members (or material) can be inserted through a concave proximal portion and a concave distal portion into the aneurysm sac (e.g. the aneurysm dome).

In an example, insertion of embolic members (or material) into a concave distal portion can expand a concave distal portion. In an example, insertion of embolic members (or material) into a concave distal portion can expand and change the shape of the concave distal portion. In an example, insertion of embolic members (or material) into a concave distal portion can expand the concave distal portion so that it conforms to the walls of even an irregularly-shaped aneurysm sac. In an example, insertion of embolic members (or material) into a concave distal portion can expand the concave distal portion in a radially-asymmetric manner, enabling it to fill a non-radially-symmetric (e.g. non-spherical) aneurysm sac. In an example, insertion of embolic members (or material) into a concave distal portion can expand the concave distal portion in an irregular manner, enabling it to fill an irregularly shaped (e.g. non-spherical) aneurysm sac. In an example, embolic members (or material) can be inserted into, and contained within, a concave distal portion, thereby expanding the concave distal portion to fill the interior of the aneurysm sac.

In an example, there can be one or more openings in a concave distal portion and a concave proximal portion through which embolic members (or material) are inserted into the concave distal portion. In an example, there can be one or more openings in a concave distal portion and a concave proximal portion along their central longitudinal axes, wherein embolic members (or material) are inserted through these one or more openings into the concave distal portion. In an example, there can be a first opening in the concave distal portion and a second opening in the concave proximal portion, wherein these first and second openings are aligned to enable insertion of embolic members (or material) through the openings into the concave distal portion. In an example, there can be an opening in a radial constraint which radially-constrains a tubular mesh from which the concave distal portion and the concave proximal portion are formed, wherein embolic members (or material) are inserted through this opening into the concave distal portion.

In an example, there can be a valve in an opening through a concave proximal portion. In an example, this valve can allow embolic members and/or material to enter a concave distal portion when the valve is open and can prevent embolic members and/or material from escaping out of the concave distal portion when the valve is closed. In an example, this valve can operate passively. In an example, a valve can be a passive one-way valve which allows embolic members and/or material to enter, but not exit, a concave distal portion or a robotic system. In another example, this valve can be actively opened or closed (remotely) by an operator who is deploying the device. In an example, a valve can be actively opened or closed by a mechanism selected from the group consisting of: application of electrical energy to the valve; pulling or pushing a wire connected to the valve; rotating a wire connected to the valve; pulling a filament or string connected to the valve; activating a small-scale electrical actuator; activating a hydraulic mechanism; activating a pneumatic mechanism; and rotating a catheter.

In an example, a method for deploying an intrasaccular aneurysm occlusion device can comprise: forming a concave distal portion of a device and a concave proximal portion of the device; inserting the concave distal portion and the concave proximal portion into an into the aneurysm sac; and inserting embolic members and/or material into the concave distal portion. In an example, a method for deploying an intrasaccular aneurysm occlusion device can comprise: forming a concave distal portion of a device and a concave proximal portion of the device by radially-constraining and inverting a tubular mesh; inserting the concave distal portion and the concave proximal portion into an into the aneurysm sac; and inserting embolic members and/or material into the concave distal portion.

In an example, a method for deploying an intrasaccular aneurysm occlusion device can comprise: inserting a concave distal portion of a device into an aneurysm sac; inserting a concave proximal portion of the device into the aneurysm sac; drawing the concave distal portion and the concave proximal portion closer together in the aneurysm sac; connecting the concave distal portion and the concave proximal portion together in the aneurysm sac; and inserting embolic members and/or material into the concave distal portion. Example variations discussed elsewhere in this disclosure or priority-linked disclosures can also be applied to this example where relevant.

The example shown in FIG. 11 is like the one in FIG. 10 except that the embolic members are “string-of-pearls” embolic strands instead of coils. FIG. 11 shows an example of an intrasaccular aneurysm occlusion device comprising: a concave distal portion 1102 which is inserted into an aneurysm sac; a concave proximal portion 1101 which is inserted into the aneurysm sac and expands to cover the aneurysm neck; and embolic members and/or material 1103 which is inserted into the concave distal portion; wherein the concave distal portion is between the concave proximal portion and the dome of the aneurysm sac, wherein at least part of the concave distal portion is nested within a concavity of the concave proximal portion, and wherein the concave distal portion is connected to the concave proximal portion at a proximal location on the concave distal portion.

More specifically, the example shown in FIG. 11 can be described as an intrasaccular aneurysm occlusion device comprising: a bowl-shaped distal portion which is inserted into an aneurysm sac; a bowl-shaped proximal portion which is inserted into the aneurysm sac and expands to cover the aneurysm neck; and “string-of-pearls” embolic strands which are inserted into the bowl-shaped distal portion; wherein the bowl-shaped distal portion is between the bowl-shaped proximal portion and the dome of the aneurysm sac, wherein between 25% and 75% of the bowl-shaped distal portion is nested within a concavity of the bowl-shaped proximal portion, and wherein the bowl-shaped distal portion is connected to the bowl-shaped proximal portion at a proximal location on the bowl-shaped distal portion.

In an example, a string-of-pearls embolic strand can be defined as a series or sequence of embolic members (e.g. beads, polyhedrons, hydrogel pieces, microsponges, or other embolic components) which are connected by flexible longitudinal members (e.g. thin wires, strands, strings, springs, sutures, ribbons, or filaments). In an example, the centers and/or centroids of embolic members (e.g. beads, polyhedrons, hydrogel pieces, microsponges, or other embolic components) in a string-of-pearls embolic strand can be connected by flexible longitudinal members (e.g. thin wires, strands, strings, springs, sutures, ribbons, or filaments). In an example, embolic members (e.g. beads, polyhedrons, hydrogel pieces, microsponges, or other embolic components) in a string-of-pearls embolic strand can be pairwise interconnected by flexible longitudinal members (e.g. thin wires, strands, strings, springs, sutures, ribbons, or filaments).

In an example, there can be variation in the size of embolic pieces along the length of a “string-of-pearls” embolic strand. In an example, embolic pieces which are closer to the distal end of a “string-of-pearls” embolic strand can be larger than embolic pieces which are father from this distal end. In an example, there can be variation in the compressibility and/or durometer of embolic pieces along the length of a “string-of-pearls” embolic strand. In an example, embolic pieces which are closer to the distal end of a “string-of-pearls” embolic strand can be less compressible and/or higher durometer than embolic pieces which are father from this distal end. In an example, there can be variation in the distance between embolic pieces along the length of a “string-of-pearls” embolic strand. In an example, embolic pieces which are closer to the distal end of a “string-of-pearls” embolic strand can be closer together than embolic pieces which are father from this distal end. Example variations discussed elsewhere in this disclosure or priority-linked disclosures can also be applied to this example where relevant.

The example shown in FIG. 12 is like the one in FIG. 10 except that embolic material is liquid or gelatinous material (e.g. a “liquid embolic”) which congeals in the aneurysm sac. FIG. 12 shows an example of an intrasaccular aneurysm occlusion device comprising: a concave distal portion 1202 which is inserted into an aneurysm sac; a concave proximal portion 1201 which is inserted into the aneurysm sac and expands to cover the aneurysm neck; and embolic members and/or material 1203 which is inserted into the concave distal portion; wherein the concave distal portion is between the concave proximal portion and the dome of the aneurysm sac, wherein at least part of the concave distal portion is nested within a concavity of the concave proximal portion, and wherein the concave distal portion is connected to the concave proximal portion at a proximal location on the concave distal portion.

More specifically, the example shown in FIG. 12 can be described as an intrasaccular aneurysm occlusion device comprising: a bowl-shaped distal portion which is inserted into an aneurysm sac; a bowl-shaped proximal portion which is inserted into the aneurysm sac and expands to cover the aneurysm neck; and congealing liquid or gelatinous embolic material which is inserted into the bowl-shaped distal portion; wherein the bowl-shaped distal portion is between the bowl-shaped proximal portion and the dome of the aneurysm sac, wherein between 25% and 75% of the bowl-shaped distal portion is nested within a concavity of the bowl-shaped proximal portion, and wherein the bowl-shaped distal portion is connected to the bowl-shaped proximal portion at a proximal location on the bowl-shaped distal portion. Example variations discussed elsewhere in this disclosure or priority-linked disclosures can also be applied to this example where relevant.

FIGS. 13 through 18 show an example of an intrasaccular aneurysm occlusion device with a limacon-shaped (e.g. cardioid or heart shaped) distal portion nested in a bowl-shaped (e.g. hemispherical, half-ellipsoidal, half-toroidal, inverted umbrella, or parabolic shape) proximal portion.

With respect to specific components, FIG. 13 shows an example of an intrasaccular aneurysm occlusion device comprising: a limacon-shaped (e.g. cardioid or heart shaped) distal portion 1302 which is inserted into an aneurysm sac; and a concave proximal portion 1301 which is inserted into the aneurysm sac and expands to cover the aneurysm neck; wherein the limacon-shaped distal portion is between the concave proximal portion and the dome of the aneurysm sac, wherein at least part of the limacon-shaped distal portion is nested within a concavity of the concave proximal portion, and wherein the limacon-shaped distal portion is connected to the concave proximal portion at a proximal location on the limacon-shaped distal portion.

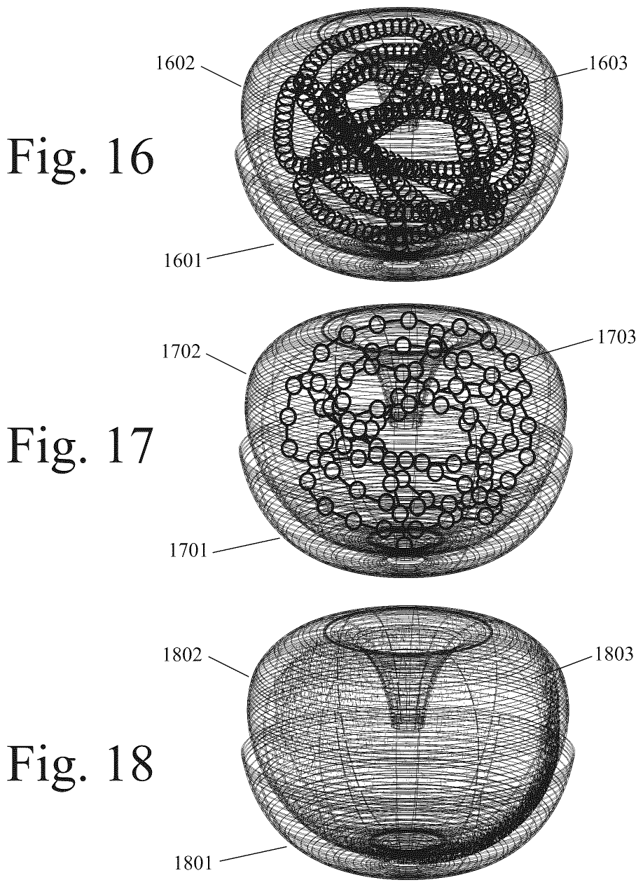

FIG. 13 shows this device without a radial constraint and without embolic members and/or material in the limacon-shaped (e.g. cardioid or heart shaped) distal portion. FIG. 14 shows this device with a radial constraint, but without embolic members and/or material in the limacon-shaped distal portion. FIG. 15 shows this device wherein the entire limacon-shaped distal portion is nested within the concavity of the concave proximal portion. FIG. 16 shows this device with embolic coils in the limacon-shaped distal portion. FIG. 17 shows this device with “string-of-pearls” embolic strands in the limacon-shaped distal portion. FIG. 18 shows this device with congealing liquid or gelatinous material in the limacon-shaped distal portion.

The example shown in FIG. 13 can also be described as an intrasaccular aneurysm occlusion device comprising: a limacon-shaped (e.g. cardioid or heart shaped) distal portion which is inserted into an aneurysm sac; and a bowl-shaped proximal portion which is inserted into the aneurysm sac and expands to cover the aneurysm neck; wherein the limacon-shaped distal portion is between the bowl-shaped proximal portion and the dome of the aneurysm sac, wherein between 25% and 75% of the limacon-shaped distal portion is nested within a concavity of the bowl-shaped proximal portion, and wherein the limacon-shaped distal portion is connected to the bowl-shaped proximal portion at a proximal location on the limacon-shaped distal portion.

In FIG. 13, a limacon-shaped (e.g. cardioid or heart shaped) distal portion has a first (proximal-to-distal) height and a concave proximal portion has a second (proximal-to-distal) height, wherein the first height is greater than the second height. In this example, the first height is at least 50% greater than the second height. In this example, between 25% and 75% of a limacon-shaped (e.g. cardioid or heart shaped) distal portion is nested within the concavity of a concave proximal portion. In this example, between 25% and 75% of a limacon-shaped (e.g. cardioid or heart shaped) distal portion overlaps a concave proximal portion. Portion width is defined herein in as a distance in a plane which is orthogonal to the proximal-to-distal axis of a portion. In this example, the concave proximal portion is wider than the limacon-shaped (e.g. cardioid or heart shaped) distal portion.

In an example, a limacon-shaped distal portion can have a cardioid or heart shape. In an example, a distal portion can have an epitrochoidal curve shape. In an example, a limacon-shaped distal portion can have a shape which is generally globular, but has an inversion and opening on its distal surface. In an example, a limacon-shaped distal portion can have a shape which is generally globular, but has a funnel or half-hyperboloidal shaped inversion and opening on its distal surface, wherein the inversion and opening extends into the interior of the distal portion, spanning the entire height of the distal portion.

In an example, a distal portion can have a shape which is generally globular, but has an indentation or dimple on its distal surface. In an example, a limacon-shaped distal portion can have a shape which is generally globular, but has an inversion and opening on its distal surface, wherein the inversion and opening extends into the interior of the distal portion, spanning the entire height of the distal portion. In an example, a limacon-shaped distal portion can have a centered trochoid curve shape.

In an example, a limacon-shaped distal portion can have a shape which is generally globular, but has an indentation or dimple on its distal surface. In an example, a limacon-shaped distal portion can have a shape which is generally globular, but has a (partial) inversion and opening on its distal surface. In an example, a distal portion can have a shape which is generally globular, but has a funnel or half-hyperboloidal shaped inversion on its distal surface, wherein the inversion extends into the interior of the distal portion, spanning between 15% and 30% of the height of the distal portion.

In an example, a limacon-shaped distal portion can have an outer sub-portion and an inner sub-portion, wherein the inner sub-portion is formed by (partially) inverting the distal end of the distal portion and wherein the inner sub-portion spans the entire central longitudinal axis of the distal portion. In an example, a limacon-shaped distal portion can have a shape which is generally globular, but has a funnel or half-hyperboloidal shaped inversion and opening on its distal surface.

In an example, a limacon-shaped distal portion can have a shape which is generally globular, but has a funnel or half-hyperboloidal shaped inversion and opening on its distal surface, wherein the inversion and opening extends into the interior of the distal portion. In an example, a limacon-shaped distal portion can have a shape which is generally globular, but has a funnel or half-hyperboloidal shaped inversion and opening on its distal surface, wherein the inversion and opening extends into the interior of the distal portion, spanning between 5% and 20% of the height of the distal portion.

In an example, a limacon-shaped distal portion can have a shape which is generally globular, but has an inversion and opening on its distal surface, wherein the inversion and opening extends into the interior of the distal portion, spanning between 5% and 20% of the height of the distal portion. In an example, a limacon-shaped distal portion can have a shape which is generally globular, but has an inversion and opening on its distal surface, wherein the inversion and opening extends into the interior of the distal portion.

In an example, a distal portion can have a shape which is generally globular, but has a funnel or half-hyperboloidal shaped inversion on its distal surface, wherein the inversion extends into the interior of the distal portion, spanning between 25% and 75% of the height of the distal portion. In an example, a distal portion can have a shape which is generally globular, but has a funnel or half-hyperboloidal shaped inversion on its distal surface, wherein the inversion extends into the interior of the distal portion, spanning the entire height of the distal portion.

In an example, a distal portion can have a bicircular rational plane algebraic curve of the fourth degree. In an example, a limacon-shaped distal portion can have a shape which is generally globular, but has an inversion and opening on its distal surface, wherein the inversion and opening extends into the interior of the distal portion, spanning between 15% and 30% of the height of the distal portion. In an example, a distal portion can have a shape which is generally globular, but has a (partial) inversion on its distal surface. In an example, a limacon-shaped distal portion can have an epitrochoidal curve shape.

In an example, a limacon-shaped distal portion can have a bicircular rational plane algebraic curve of the fourth degree. In an example, a distal portion can have a shape which is generally globular, but has a funnel or half-hyperboloidal shaped inversion on its distal surface. In an example, a limacon-shaped distal portion can have an outer sub-portion and an inner sub-portion, wherein the inner sub-portion is formed by (partially) inverting the distal end of the distal portion.

In an example, a distal portion can have a shape which is generally globular, but has a funnel or half-hyperboloidal shaped inversion on its distal surface, wherein the inversion extends into the interior of the distal portion, spanning between 5% and 20% of the height of the distal portion. In an example, a limacon-shaped distal portion can have an outer sub-portion and an inner sub-portion, wherein the inner sub-portion is formed by (partially) inverting the distal end of the distal portion and wherein the inner sub-portion spans between 25% and 75% of the central longitudinal axis of the distal portion.

In an example, a limacon-shaped distal portion can have a shape which is generally globular, but has an inversion and opening on its distal surface. In an example, a distal portion can have a centered trochoid curve shape. In an example, a limacon-shaped distal portion can have a shape which is generally globular, but has an inversion and opening on its distal surface, wherein the inversion and opening extends into the interior of the distal portion, spanning between 25% and 75% of the height of the distal portion.

In an example, a limacon-shaped distal portion can have a shape which is generally globular, but has a funnel or half-hyperboloidal shaped inversion and opening on its distal surface, wherein the inversion and opening extends into the interior of the distal portion, spanning between 25% and 75% of the height of the distal portion. In an example, a distal portion can have a shape which is generally globular, but has a funnel or half-hyperboloidal shaped inversion on its distal surface, wherein the inversion extends into the interior of the distal portion.

In an example, a limacon-shaped distal portion can have an outer sub-portion and an inner sub-portion, wherein the inner sub-portion is formed by (partially) inverting the distal end of the distal portion and wherein the inner sub-portion spans between 10% and 30% of the central longitudinal axis of the distal portion. In an example, a limacon-shaped distal portion can have a shape which is generally globular, but has a funnel or half-hyperboloidal shaped inversion and opening on its distal surface, wherein the inversion and opening extends into the interior of the distal portion, spanning between 15% and 30% of the height of the distal portion.

In an example, a limacon-shaped (e.g. cardioid or heart shaped) distal portion can be more flexible than the concave proximal portion. In an example, the shape of a limacon-shaped distal portion can change if embolic members and/or material are inserted into it. In an example, a limacon-shaped distal portion can conform to the irregular shape of the walls of an irregularly-shaped aneurysm sac after insertion of embolic members and/or material into the limacon-shaped distal portion. In an example, the limacon-shaped distal portion can be a flexible net or mesh.

In this example, the concave proximal portion has a bowl shape. In various examples, a concave proximal portion can have a shape which is selected from the group consisting of: hemispherical, half-ellipsoidal, half-toroidal (e.g. lower half), inverted umbrella shape, inverted dome shape, parabolic, carlavian curve, and conic section. In an example, a concave proximal portion can have a radially-compressed configuration as it is delivered through a catheter into an aneurysm sac. In an example, a concave proximal portion can self-expand radially after it has been inserted into the aneurysm sac. In an example, a concave proximal portion can be expanded radially after it has been inserted into the aneurysm sac by moving a wire to which it is attached or by the application of electrical energy.

In an example, a distal end of a limacon-shaped (e.g. cardioid or heart shaped) distal portion can radially-constrained and inverted. In an example, a distal end of a limacon-shaped (e.g. cardioid or heart shaped) distal portion can radially-constrained by an annular ring, band, or wire and inverted. In another example, a distal end of a limacon-shaped (e.g. cardioid or heart shaped) distal portion can be inverted and protrude into the interior of the limacon-shaped (e.g. cardioid or heart shaped) distal portion.

In an example, there can be a local concavity at a distal end of a limacon-shaped (e.g. cardioid or heart shaped) distal portion. In an example, there can be a local concavity at a distal end of a limacon-shaped (e.g. cardioid or heart shaped) distal portion, wherein the width of the local concavity is between 10% and 30% of the maximum width of the limacon-shaped (e.g. cardioid or heart shaped) distal portion. In an example, there can be a local concavity at a distal end of a limacon-shaped (e.g. cardioid or heart shaped) distal portion, wherein the width of the local concavity is between 20% and 40% of the maximum width of the limacon-shaped (e.g. cardioid or heart shaped) distal portion.

In an example, a distal end of a limacon-shaped (e.g. cardioid or heart shaped) distal portion can be radially-constrained (e.g. pinched). In an example, a distal end of a limacon-shaped (e.g. cardioid or heart shaped) distal portion can be radially-constrained (e.g. pinched) closed by an annular ring, band, wire, string, or cylinder. In an example, the distal end of a limacon-shaped (e.g. cardioid or heart shaped) distal portion may be closed (e.g. pinched and/or radially-constrained) by a radial constraint (e.g. an annular ring, band, or wire).

In this example, except for the connection between the concave proximal portion and the limacon-shaped (e.g. cardioid or heart shaped) distal portion, there is a gap between the concave proximal portion and the limacon-shaped distal portion. In an example, this can be a uniform gap (e.g. a gap with a constant distance). In an example, a gap between a concave proximal portion and a limacon-shaped distal portion where they are nested together can be a uniform gap (e.g. constant distance). In another example, this gap can decrease with greater distance from the connection between the concave proximal portion and the limacon-shaped distal portion. In another example, this gap can increase with greater distance from the connection between the concave proximal portion and the limacon-shaped distal portion. In another example, this gap can first decrease and then increase with greater distance from the connection between the concave proximal portion and the limacon-shaped distal portion. In another example, this gap can first increase and then decrease with greater distance from the connection between the concave proximal portion and the limacon-shaped distal portion.

In an example, a distal circumference of a concave proximal portion can fit snuggly around the circumference of a limacon-shaped distal portion, but there can be a gap between the concave proximal portion and the limacon-shaped distal portion closer to wherein they are connected at their proximal ends (except for the connection itself). In an example, there can be a first average distance between a concave proximal portion and a limacon-shaped distal portion in the most proximal quartile of the limacon-shaped distal portion and a second average distance between the concave proximal portion and the limacon-shaped distal portion in the proximal-to-middle quartile, wherein the second distance is less than the first distance. In an example, there can be a first average distance between a concave proximal portion and a limacon-shaped distal portion in the most proximal quartile of the limacon-shaped distal portion and a second average distance between the concave proximal portion and the limacon-shaped distal portion in the proximal-to-middle quartile, wherein the second distance is less than half of the first distance.

In this example, the limacon-shaped (e.g. cardioid or heart shaped) distal portion and the concave proximal portion are coaxial as well as being nested. In this example, at least a portion of their central proximal-to-distal axes overlap. In this example, a limacon-shaped distal portion and a concave proximal portion are connected around their central proximal-to-distal axes. In this example, a limacon-shaped distal portion and a concave proximal portion are radially-symmetric around their central proximal-to-distal axes. In an example, a limacon-shaped distal portion can have a first, unexpanded, configuration in which it is radially symmetric and a second, expanded, configuration in which it is not radially symmetric.

In this example, a cross-section (in a plane which is orthogonal to a proximal-to-distal axis) of a limacon-shaped (e.g. cardioid or heart shaped) distal portion has circular perimeter. In an example, a cross-section (in a plane which is orthogonal to a proximal-to-distal axis) of a limacon-shaped (e.g. cardioid or heart shaped) distal portion can have an elliptical or oval perimeter. In another example, a cross-section (in a plane which is orthogonal to a proximal-to-distal axis) of a limacon-shaped (e.g. cardioid or heart shaped) distal portion can have an undulating (e.g. sinusoidal) and/or lobed perimeter.

In this example, the limacon-shaped (e.g. cardioid or heart shaped) distal portion and the concave proximal portion are portions of the same continuous member. In this example, the limacon-shaped distal portion and the concave proximal portion are made by radially-constraining and/or inverting (or everting) a single continuous member. In this example, the limacon-shaped distal portion and the concave proximal portion are made by radially-constraining and/or inverting (or everting) a continuous tubular mesh. In an example, a limacon-shaped distal portion and a concave proximal portion can be formed by radially-constraining and/or inverting (or everting) a tubular wire mesh or braid.

In an example, a limacon-shaped (e.g. cardioid or heart shaped) distal portion and a concave proximal portion can be formed by radially-constraining a tubular mesh (or braid) at one or more points along its longitudinal axis and then inverting (or everting) the tubular mesh (or braid). In an example, a limacon-shaped distal portion and a concave proximal portion can be formed by radially-constraining a tubular mesh (or braid) at one or both ends and then inverting (or everting) the tubular mesh (or braid). In an example, a limacon-shaped distal portion and a concave proximal portion can be formed by radially-constraining a tubular mesh (or braid) at two locations along its longitudinal axis. In an example, a limacon-shaped distal portion and a concave proximal portion can be formed by inverting a tubular mesh (or braid) at two locations along its longitudinal axis.

In an example, a limacon-shaped (e.g. cardioid or heart shaped) distal portion and a concave proximal portion can be formed by radially-constraining a tubular mesh (or braid) at one or both ends, radially constraining the tubular mesh (or braid) at a middle point (between the ends), and then inverting (or everting) the tubular mesh (or braid). In an example, a limacon-shaped distal portion can be made from metal (e.g. gold). Limacons are known for hiding pots of gold. Such devices could be a lucky charms for the recipients. In an example, a limacon-shaped (e.g. cardioid or heart shaped) distal portion and a concave proximal portion can be formed by radially-constraining a tubular mesh (or braid) at one or both ends, radially constraining the tubular mesh (or braid) at a middle point (between the ends), and then inverting (or everting) the tubular mesh (or braid) multiple times.

In an example, a limacon-shaped (e.g. cardioid or heart shaped) distal portion and a concave proximal portion can be formed by radially-constraining a tubular mesh (or braid) at multiple points along its longitudinal axis. In an example, a limacon-shaped distal portion and a concave proximal portion can be formed by inverting a tubular mesh (or braid) at multiple points along its longitudinal axis. In an example, a limacon-shaped distal portion and a concave proximal portion can be formed by radially-constraining and inverting a tubular mesh (or braid) at multiple points along its longitudinal axis. In an example, a limacon-shaped distal portion and a concave proximal portion can be formed by radially-constraining a tubular mesh (or braid) at two locations along its longitudinal axis. In an example, a limacon-shaped distal portion and a concave proximal portion can be formed by inverting a tubular mesh (or braid) at two locations along its longitudinal axis.

In an example, a limacon-shaped (e.g. cardioid or heart shaped) distal portion and a concave proximal portion can be formed by radially-constraining and inverting (or everting) a tubular member (e.g. tubular mesh or braid). In an example, a tubular member can be radially-constrained by a radial constraint which is selected from the group consisting of: annular ring, band, cylinder, or washer; circular wire, strap, filament, or string; helical wire or spring.

In an example, a radial constraint can be a compound component with an outer part and an inner part. In an example, the outer and inner parts can be nested, coaxial, and/or concentric. In an example, a radial-constraint can include an outer part (on the outside of the tubular member) and an inner part (on the inside of the tubular member), wherein the tubular member is held between (e.g. passes between) the outer part and the inner part. In an example, there can be an opening through the inner part through which embolic members and/or material is inserted into the limacon-shaped distal portion.

In an example, a radial-constraint can include two nested (e.g. coaxial or concentric) bands, rings, or cylinders comprising: an outer band, ring, or cylinder (on the outside of the tubular member) and an inner band, ring, or cylinder (on the inside of the tubular member), wherein the tubular member is held between (e.g. passes through) the outer band, ring, or cylinder and the inner band, ring, or cylinder, and wherein embolic members (or material) are inserted in the limacon-shaped distal portion through an opening (e.g. lumen) the inner band, ring, or cylinder.

In an example, a limacon-shaped (e.g. cardioid or heart shaped) distal portion and a concave proximal portion can be formed from a tubular member (e.g. wire mesh or braid), wherein the tubular member has longitudinal variation in width, wall thickness, wire thickness, braid or weave density, type or thickness of coating, and material composition. In an example, this tubular mesh can be tapered. In an example, a proximal portion of the tubular mesh can be more dense, stiffer, thicker, and/or less porous than a distal portion of the tubular mesh. In an example, a proximal portion of the tubular mesh can be coated to make it more dense, stiffer, thicker, and/or less porous than a distal portion of the tubular mesh.

In this example, the limacon-shaped (e.g. cardioid or heart shaped) distal portion and the concave proximal portion are made from the same continuous piece of material (e.g. a tubular mesh). In another example, a limacon-shaped distal portion and a concave proximal portion can be made separately and then connected together. In another example, a limacon-shaped distal portion and a concave proximal portion can be made from different materials (and/or with different processes) and then connected together. In another example, a limacon-shaped distal portion and a concave proximal portion can be made from different materials (and/or with different processes) and then connected together before they are inserted into a catheter for deliver into an aneurysm sac. In another example, a limacon-shaped distal portion and a concave proximal portion can be made from different materials (and/or with different processes) and then connected after they have been inserted into an aneurysm sac.

In an example, a concave proximal portion can be less elastic (e.g. made with less elastic material) than a limacon-shaped (e.g. cardioid or heart shaped) distal portion. In an example, the concave proximal portion can be thicker (e.g. made with thicker wires) than the limacon-shaped distal portion. In an example, the concave proximal portion can be less compressible (e.g. made with higher durometer material) than the limacon-shaped distal portion. In an example, the concave proximal portion can be denser (e.g. made with a denser braid or weave) than the limacon-shaped distal portion. In an example, the concave proximal portion can be less porous (e.g. made with a liquid-impermeable layer) than the limacon-shaped distal portion. In an example, the concave proximal portion can have more layers (e.g. two or three layers) than the limacon-shaped distal portion. In an example, the concave proximal portion can comprise a liquid-impermeable (e.g. polymer membrane) layer between two wire layers.

In an example, a limacon-shaped (e.g. cardioid or heart shaped) distal portion can be more elastic (e.g. made with more elastic material) than a concave proximal portion. In an example, the limacon-shaped distal portion can be thinner (e.g. made with thinner wires) than the concave proximal portion. In an example, the limacon-shaped distal portion can be more compressible (e.g. made with lower durometer material) than the concave proximal portion. In an example, the limacon-shaped distal portion can be less dense (e.g. made with a less dense braid or weave) than the concave proximal portion. In an example, the limacon-shaped distal portion can be more porous (e.g. made with larger holes) than the concave proximal portion. In an example, the limacon-shaped distal portion can have fewer layers than the concave proximal portion.

In this example, the concave proximal portion has one layer. In another example, a concave proximal portion can have two layers. In another example, the concave proximal portion can have two layers which are formed by radially-constraining a tubular mesh at its proximal end and a middle location, and then inverting (or everting) the area of the tubular mesh between the proximal end and the middle location from a single layer to a double layer. In another example, a concave proximal portion can have three layers, comprising two wire mesh layers and a membrane between the wire mesh layers. In an example, a concave proximal portion can have multiple layers, but a limacon-shaped (e.g. cardioid or heart shaped) distal portion has only one layer. In an example, a concave proximal portion can have more layers than a limacon-shaped (e.g. cardioid or heart shaped) distal portion.

In an example, a limacon-shaped (e.g. cardioid or heart shaped) distal portion and a concave proximal portion can both be made from wire. In an example, a limacon-shaped distal portion and a concave proximal portion can both be made by braiding or weaving wires. In an example, a limacon-shaped distal portion and a concave proximal portion can be made by braiding or weaving nitinol wires. In another example, a limacon-shaped distal portion and a concave proximal portion can be made by 3D printing. In an example, a limacon-shaped distal portion can be made from a polymer and a concave proximal portion can be made from metal. In an example, a limacon-shaped distal portion can be a polymer mesh or net and a concave proximal portion can be a braid or weave of metal wires. In an example, a limacon-shaped distal portion can be a flexible polymer net or mesh and a concave proximal portion can be a wire mesh or stent.

In an example, a limacon-shaped (e.g. cardioid or heart shaped) distal portion and a concave proximal portion can be attached to each other before they are delivered through a catheter into an aneurysm sac. In an example, a limacon-shaped distal portion and a concave proximal portion can be inserted into an aneurysm sac at substantially the same time. In an example, a limacon-shaped distal portion and a concave proximal portion can be nested (e.g. overlap) before and after they are inserted into an aneurysm. In another example, a limacon-shaped distal portion and a concave proximal portion may not be nested (e.g. not overlap) while they are being delivered through a catheter into an aneurysm sac, but are connected to each other within the aneurysm sac so that they become nested (e.g. overlapping) in the aneurysm sac.

In an example, a limacon-shaped (e.g. cardioid or heart shaped) distal portion and a concave proximal portion can be delivered through a catheter sequentially so that they do not overlap while they are being delivered through the catheter. In an example, a limacon-shaped (e.g. cardioid or heart shaped) distal portion and a concave proximal portion can be separated by a longitudinal distance as they are being delivered through a catheter sequentially, but are moved closer to each other (and connected) after they have been inserted into an aneurysm sac.

In an example, a limacon-shaped (e.g. cardioid or heart shaped) distal portion and a concave proximal portion are not nested as they are delivered sequentially through a catheter into an aneurysm sac, but are nested after they are connected to each other within the aneurysm sac. In an example, a limacon-shaped (e.g. cardioid or heart shaped) distal portion and a concave proximal portion do not overlap as they are delivered sequentially through a catheter into an aneurysm sac, but do overlap after they are connected to each other within the aneurysm sac.

In an example, a method for deploying an intrasaccular aneurysm occlusion device can comprise: inserting a limacon-shaped (e.g. cardioid or heart shaped) distal portion of a device into an aneurysm sac; inserting a concave proximal portion of the device into the aneurysm sac; drawing the limacon-shaped distal portion and the concave proximal portion closer together in the aneurysm sac; and connecting the limacon-shaped distal portion and the concave proximal portion together in the aneurysm sac.

In an example, a limacon-shaped (e.g. cardioid or heart shaped) distal portion and a concave proximal portion can be separated by a longitudinal distance as they are being delivered through a catheter sequentially, but are moved closer to each other (and connected) by movement of a wire (connected to one or both of them) after they have been inserted into an aneurysm sac. In an example, this device can further comprise a wire which is attached to the limacon-shaped (e.g. cardioid or heart shaped) distal portion. In an example, this device can further comprise a wire which is attached to the distal end of the limacon-shaped (e.g. cardioid or heart shaped) distal portion, wherein the limacon-shaped (e.g. cardioid or heart shaped) distal portion is drawn closer to the concave proximal portion when the wire is pulled.

In an example, this device can further comprise a wire which is attached to the concave proximal portion. In an example, this device can further comprise a wire which is attached to the concave proximal portion, wherein the concave proximal portion is drawn closer to the limacon-shaped distal portion when the wire is pushed. In an example, this device can further comprise a two wires, one wire which is attached to the limacon-shaped distal portion and one wire which is attached to the concave proximal portion, wherein the pulling or pushing one or both wires moves the two portions closer together.

In an example, a limacon-shaped (e.g. cardioid or heart shaped) distal portion and a concave proximal portion can be separated by a longitudinal distance as they are being delivered through a catheter sequentially, but are moved closer to each other (and connected) by application of electricity (to one or both of them) after they have been inserted into an aneurysm sac.

In an example, a limacon-shaped distal portion of this device can be a flexible sac-filling portion of this device. In an example, a limacon-shaped distal portion of this device can be a distal mesh. In an example, a limacon-shaped distal portion or distal mesh can have a cardioid shape.

In an example, an intrasaccular aneurysm occlusion device can comprise: a distal mesh (or net or porous balloon) which is inserted into an aneurysm sac; wherein the distal mesh is cardioid, crescent shaped, and/or kidney shaped; wherein the distal mesh centroid is a first distance from the aneurysm neck; and a bowl-shaped proximal mesh inserted into the aneurysm sac; wherein the proximal mesh centroid is a second distance from the aneurysm neck; wherein the second distance is less than the first distance; wherein the proximal mesh spans the aneurysm neck; wherein a portion of the distal mesh is within the concavity of the proximal mesh; wherein the proximal mesh is axially-aligned with the distal mesh; and wherein the distal mesh and/or proximal mesh are filled with embolic material (such as microsponges, hydrogels, or coils).

In an example, an intrasaccular aneurysm occlusion device can comprise: a distal mesh which is inserted into an aneurysm sac, wherein the distal mesh is cardioid shaped, and wherein the distal mesh centroid is a first distance from the aneurysm neck; and a bowl-shaped proximal mesh inserted into the aneurysm sac, wherein the proximal mesh centroid is a second distance from the aneurysm neck, wherein the second distance is less than the first distance, wherein a portion of the distal mesh is within a concavity of the proximal mesh, and wherein the distal mesh is filled with embolic material.

In an example, an intrasaccular aneurysm occlusion device can comprise: a distal portion and/or mesh which is inserted into an aneurysm sac, wherein the distal portion and/or mesh is cardioid shaped, and wherein the distal portion and/or mesh centroid is a first distance from the aneurysm neck; and a bowl-shaped proximal portion and/or mesh inserted into the aneurysm sac, wherein the proximal portion and/or mesh centroid is a second distance from the aneurysm neck, wherein the second distance is less than the first distance, wherein a portion of the distal portion and/or mesh is within a concavity of the proximal portion and/or mesh, and wherein the distal portion and/or mesh is filled with embolic material.

In an example, an intrasaccular aneurysm occlusion device can comprise: a limacon and/or cardioid shaped distal portion or mesh which is inserted into an aneurysm sac, wherein the limacon and/or cardioid shaped distal portion or mesh centroid is a first distance from the aneurysm neck; and a bowl-shaped proximal portion or mesh inserted into the aneurysm sac, wherein the bowl-shaped proximal portion or mesh centroid is a second distance from the aneurysm neck, wherein the second distance is less than the first distance, wherein a portion of the limacon and/or cardioid shaped distal portion or mesh is within a concavity of the bowl-shaped proximal portion or mesh, and wherein the limacon and/or cardioid shaped distal portion or mesh is filled with embolic material.

In an example, an intrasaccular aneurysm occlusion device can comprise: a cardioid-shaped distal mesh which is inserted into an aneurysm sac, wherein the cardioid-shaped distal mesh centroid is a first distance from the aneurysm neck; and a bowl-shaped proximal mesh inserted into the aneurysm sac, wherein the bowl-shaped proximal mesh centroid is a second distance from the aneurysm neck, wherein the second distance is less than the first distance, wherein a portion of the cardioid-shaped distal mesh is within a concavity of the bowl-shaped proximal mesh, and wherein the cardioid-shaped distal mesh is filled with embolic material.

In an example, an intrasaccular aneurysm occlusion device can comprise: a cardioid-shaped distal mesh which is inserted into an aneurysm sac, wherein the cardioid-shaped distal mesh centroid is a first distance from the aneurysm neck; and a bowl-shaped proximal mesh inserted into the aneurysm sac, wherein the bowl-shaped proximal mesh centroid is a second distance from the aneurysm neck, wherein the second distance is less than the first distance, and wherein a portion of the cardioid-shaped distal mesh is within a concavity of the bowl-shaped proximal mesh.

In an example, an aneurysm occlusion device can comprise: a distal (wire and/or polymer) mesh inserted into an aneurysm sac; wherein the distal mesh is cardioid shaped; wherein the distal mesh centroid is a first distance from the aneurysm neck; and a proximal mesh inserted into the aneurysm sac; wherein the proximal mesh centroid is a second distance from the aneurysm neck; wherein the second distance is less than the first distance; wherein the proximal mesh spans the aneurysm neck; wherein the proximal mesh is axially-aligned with the distal mesh; and wherein the distal mesh and/or proximal mesh are filled with embolic material (such as microsponges, hydrogels, or coils).

In an example, an aneurysm occlusion device can comprise: a distal mesh (or net or porous balloon) which is inserted into an aneurysm sac; wherein the distal mesh is cardioid shaped; wherein the distal mesh centroid is a first distance from the aneurysm neck; and a bowl-shaped proximal mesh inserted into the aneurysm sac; wherein the proximal mesh centroid is a second distance from the aneurysm neck; wherein the second distance is less than the first distance; wherein the proximal mesh spans the aneurysm neck; wherein a portion of the distal mesh is within the concavity of the proximal mesh; wherein the proximal mesh is axially-aligned with the distal mesh; and wherein the distal mesh and/or proximal mesh are filled with embolic material (such as microsponges, hydrogels, or coils).

In an example, an intrasaccular aneurysm occlusion device can comprise: an outer bowl-shaped (e.g. hemispherical, half-ellipsoidal, half-toroidal, or parabolic) portion; a middle globular (e.g. spherical, ellipsoidal, oblate spherical, or apple-shaped) portion; and an inner funnel-shaped (e.g. hyperboloidal or half-hyperboloidal) portion; wherein the middle globular-shaped portion is between the outer bowl-shaped portion and the inner funnel-shaped portion, wherein the middle globular-shaped portion is at least partially nested within the bowl-shaped portion, and wherein the distal opening of the inner funnel-shaped portion is larger than the proximal opening of the inner funnel-shaped portion. In an example, the funnel-shaped portion, the middle globular portion, and the outer bowl-shaped portion can be coaxial. In an example, the funnel-shaped portion can span the entire central proximal-to-distal axes of the middle globular portion and/or the outer bowl-shaped portion. In an example, the middle globular portion can be filled with embolic members and/or embolic material. In an example, embolic members and/or embolic material can be inserted into an aneurysm sac through the inner funnel-shaped portion.

In an example, an intrasaccular aneurysm occlusion device can comprise: an outer bowl-shaped (e.g. hemispherical, half-ellipsoidal, half-toroidal, or parabolic) portion; a middle limacon-shaped (e.g. cardioid-shaped) portion; and an inner funnel-shaped (e.g. hyperboloidal or half-hyperboloidal) portion; wherein the middle limacon-shaped (e.g. cardioid-shaped) portion is between the outer bowl-shaped portion and the inner funnel-shaped portion, wherein the middle limacon-shaped (e.g. cardioid-shaped) portion is at least partially nested within the bowl-shaped portion, and wherein the distal opening of the inner funnel-shaped portion is larger than the proximal opening of the inner funnel-shaped portion. In an example, the funnel-shaped portion, the middle limacon-shaped (e.g. cardioid-shaped) portion, and the outer bowl-shaped portion can be coaxial. In an example, the funnel-shaped portion can span the entire central proximal-to-distal axes of the middle limacon-shaped (e.g. cardioid-shaped) portion and/or the outer bowl-shaped portion. In an example, the middle limacon-shaped (e.g. cardioid-shaped) portion can be filled with embolic members and/or embolic material. In an example, embolic members and/or embolic material can be inserted into an aneurysm sac through the inner funnel-shaped portion.

In an example, an intrasaccular aneurysm occlusion device can comprise: an outer bowl-shaped (e.g. hemispherical, half-ellipsoidal, half-toroidal, or parabolic) portion; a middle cardioid-shaped (e.g. heart-shaped) portion; and an inner funnel-shaped (e.g. hyperboloidal or half-hyperboloidal) portion; wherein the middle cardioid-shaped (e.g. heart-shaped) portion is between the outer bowl-shaped portion and the inner funnel-shaped portion, wherein the middle cardioid-shaped (e.g. heart-shaped) portion is at least partially nested within the bowl-shaped portion, and wherein the distal opening of the inner funnel-shaped portion is larger than the proximal opening of the inner funnel-shaped portion. In an example, the funnel-shaped portion, the middle cardioid-shaped (e.g. heart-shaped) portion, and the outer bowl-shaped portion can be coaxial. In an example, the funnel-shaped portion can span the entire central proximal-to-distal axes of the middle cardioid-shaped (e.g. heart-shaped) portion and/or the outer bowl-shaped portion. In an example, the middle cardioid-shaped (e.g. heart-shaped) portion can be filled with embolic members and/or embolic material. In an example, embolic members and/or embolic material can be inserted into an aneurysm sac through the inner funnel-shaped portion.

In an example, an intrasaccular aneurysm occlusion device can comprise: an outer bowl-shaped (e.g. hemispherical, half-ellipsoidal, half-toroidal, or parabolic) mesh; a middle globular (e.g. spherical, ellipsoidal, oblate spherical, or apple shaped) mesh; and an inner funnel-shaped (e.g. hyperboloidal or half-hyperboloidal) mesh; wherein the middle globular-shaped mesh is between the outer bowl-shaped mesh and the inner funnel-shaped mesh, wherein the middle globular-shaped mesh is at least partially nested within the bowl-shaped mesh, and wherein the distal opening of the inner funnel shaped mesh is larger than the proximal opening of the inner funnel-shaped mesh. In an example, the funnel-shaped mesh, the middle globular mesh, and the outer bowl-shaped mesh can be coaxial. In an example, the funnel-shaped mesh can span the entire central proximal-to-distal axes of the middle globular mesh and/or the outer bowl-shaped mesh. In an example, the middle globular mesh can be filled with embolic members and/or embolic material. In an example, embolic members and/or embolic material can be inserted into an aneurysm sac through the inner funnel-shaped mesh.

In an example, an intrasaccular aneurysm occlusion device can comprise: an outer concave (e.g. hemispherical, half-ellipsoidal, half-toroidal, or parabolic) portion; a middle convex (e.g. spherical, ellipsoidal, oblate spherical, or apple shaped) portion; and an inner funnel-shaped (e.g. hyperboloidal or half-hyperboloidal) portion; wherein the middle convex portion is between the outer concave portion and the inner funnel-shaped portion, wherein the middle convex portion is at least partially nested within the outer concave portion, and wherein the distal opening of the inner funnel-shaped portion is larger than the proximal opening of the inner funnel-shaped portion. In an example, the funnel-shaped portion, the middle convex portion, and the outer concave portion can be coaxial. In an example, the funnel-shaped portion can span the entire central proximal-to-distal axis of the middle convex portion and/or the outer concave portion. In an example, the middle convex portion can be filled with embolic members and/or embolic material. In an example, embolic members and/or embolic material can be inserted into an aneurysm sac through the inner funnel-shaped portion. Relevant example variations discussed elsewhere in this disclosure or priority-linked disclosures can also be applied to the example shown in FIG. 13.

The example shown in FIG. 14 is like the one in FIG. 13 except that it includes a radial constraint (e.g. an annular ring, band, cylinder, or wire) around the connection between the limacon-shaped (e.g. cardioid or heart shaped) distal portion and the concave proximal portion. FIG. 14 shows an example of an intrasaccular aneurysm occlusion device comprising: a limacon-shaped (e.g. cardioid or heart shaped) distal portion 1402 which is inserted into an aneurysm sac; a concave proximal portion 1401 which is inserted into the aneurysm sac and expands to cover the aneurysm neck; and an annular constraint 1403 which radially constrains the connection between the limacon-shaped distal portion and the concave proximal portion; wherein the limacon-shaped distal portion is between the concave proximal portion and the dome of the aneurysm sac, wherein at least part of the limacon-shaped distal portion is nested within a concavity of the concave proximal portion, and wherein the limacon-shaped distal portion is connected to the concave proximal portion at a proximal location on the limacon-shaped distal portion.

More specifically, the example shown in FIG. 14 can be described as an intrasaccular aneurysm occlusion device comprising: a limacon-shaped (e.g. cardioid or heart shaped) distal portion which is inserted into an aneurysm sac; a bowl-shaped proximal portion which is inserted into the aneurysm sac and expands to cover the aneurysm neck; and a radial constraint around the connection between the limacon-shaped distal portion and the concave proximal portion; wherein the limacon-shaped distal portion is between the bowl-shaped proximal portion and the dome of the aneurysm sac, wherein between 25% and 75% of the limacon-shaped distal portion is nested within a concavity of the bowl-shaped proximal portion, and wherein the limacon-shaped distal portion is connected to the bowl-shaped proximal portion at a proximal location on the limacon-shaped distal portion. Example variations discussed elsewhere in this disclosure or priority-linked disclosures can also be applied to this example where relevant.

The example shown in FIG. 15 is like the one in FIG. 13 except that the height of the concave distal portion is greater than the height of the concave proximal portion. In other words, the entire limacon-shaped (e.g. cardioid or heart shaped) distal portion is nested within the concave proximal portion. FIG. 15 shows an example of an intrasaccular aneurysm occlusion device comprising: a limacon-shaped distal portion 1502 which is inserted into an aneurysm sac; and a concave proximal portion 1501 which is inserted into the aneurysm sac and expands to cover the aneurysm neck; wherein the limacon-shaped distal portion is between the proximal surface of the concave proximal portion and the dome of the aneurysm sac, wherein the entire limacon-shaped distal portion is nested within a concavity of the concave proximal portion, and wherein the limacon-shaped distal portion is connected to the concave proximal portion at a proximal location on the limacon-shaped distal portion.

More specifically, the example shown in FIG. 15 can be described as an intrasaccular aneurysm occlusion device comprising: a limacon-shaped (e.g. cardioid or heart shaped) distal portion which is inserted into an aneurysm sac; and a bowl-shaped proximal portion which is inserted into the aneurysm sac and expands to cover the aneurysm neck; wherein the limacon-shaped distal portion is between the proximal surface of the bowl-shaped proximal portion and the dome of the aneurysm sac, wherein the entire limacon-shaped distal portion is nested within a concavity of the bowl-shaped proximal portion, and wherein the limacon-shaped distal portion is connected to the bowl-shaped proximal portion at a proximal location on the limacon-shaped distal portion. In an example, such a device can be shaped like pascal's snail in a pail. If it then holds embolic coils in the sac, then this can be jail by pascal's snail in a pail.

In this example, the limacon-shaped (e.g. cardioid or heart shaped) distal portion has a first (proximal-to-distal) height and the concave proximal portion has a second (proximal-to-distal height), wherein the second height is greater than the first height. In this example, the limacon-shaped (e.g. cardioid or heart shaped) distal portion has a first height and the concave proximal portion has a second height, wherein the second height is at least 50% greater than the first height. In this example, the entire limacon-shaped (e.g. cardioid or heart shaped) distal portion is nested within the concavity of the concave proximal portion. Example variations discussed elsewhere in this disclosure or priority-linked disclosures can also be applied to this example where relevant.

The example shown in FIG. 16 is like the one in FIG. 13 except that embolic members and/or material have been inserted into the limacon-shaped (e.g. cardioid or heart shaped) distal portion. In this example, the embolic members are coils. FIG. 16 shows an example of an intrasaccular aneurysm occlusion device comprising: a limacon-shaped distal portion 1602 which is inserted into an aneurysm sac; a concave proximal portion 1601 which is inserted into the aneurysm sac and expands to cover the aneurysm neck; and embolic members and/or material 1603 which is inserted into the limacon-shaped distal portion; wherein the limacon-shaped distal portion is between the concave proximal portion and the dome of the aneurysm sac, wherein at least part of the limacon-shaped distal portion is nested within a concavity of the concave proximal portion, and wherein the limacon-shaped distal portion is connected to the concave proximal portion at a proximal location on the limacon-shaped distal portion.

More specifically, the example shown in FIG. 16 can be described as an intrasaccular aneurysm occlusion device comprising: a limacon-shaped (e.g. cardioid or heart shaped) distal portion which is inserted into an aneurysm sac; a bowl-shaped proximal portion which is inserted into the aneurysm sac and expands to cover the aneurysm neck; and embolic coils which are inserted into the limacon-shaped distal portion; wherein the limacon-shaped distal portion is between the bowl-shaped proximal portion and the dome of the aneurysm sac, wherein between 25% and 75% of the limacon-shaped distal portion is nested within a concavity of the bowl-shaped proximal portion, and wherein the limacon-shaped distal portion is connected to the bowl-shaped proximal portion at a proximal location on the limacon-shaped distal portion.

In an example, an intrasaccular aneurysm occlusion device can comprise: a limacon-shaped (e.g. cardioid or heart shaped) distal portion which is inserted into an aneurysm sac; a concave proximal portion which is inserted into the aneurysm sac and expands to cover the aneurysm neck; and embolic members and/or material; wherein the limacon-shaped distal portion is between the concave proximal portion and the dome of the aneurysm sac, wherein at least part of the limacon-shaped distal portion is nested within a concavity of the concave proximal portion, wherein the limacon-shaped distal portion is connected to the concave proximal portion at a proximal location on the limacon-shaped distal portion, and wherein the embolic members and/or material are inserted through the concave proximal portion into the limacon-shaped distal portion.

In an example, an intrasaccular aneurysm occlusion device can comprise: a limacon-shaped (e.g. cardioid or heart shaped) distal portion which is inserted into an aneurysm sac; a concave proximal portion which is inserted into the aneurysm sac and expands to cover the aneurysm neck; and embolic members and/or material; wherein the limacon-shaped distal portion is between the concave proximal portion and the dome of the aneurysm sac, wherein at least part of the limacon-shaped distal portion is nested within a concavity of the concave proximal portion, wherein the limacon-shaped distal portion is connected to the concave proximal portion at a proximal location on the limacon-shaped distal portion, and wherein the embolic members and/or material are inserted through an opening in concave proximal portion into the limacon-shaped distal portion.

In another example, an intrasaccular aneurysm occlusion device can comprise: a limacon-shaped (e.g. cardioid or heart shaped) distal portion which is inserted into an aneurysm sac; a concave proximal portion which is inserted into the aneurysm sac and expands to cover the aneurysm neck; and embolic members and/or material; wherein the limacon-shaped distal portion is between the concave proximal portion and the dome of the aneurysm sac, wherein at least part of the limacon-shaped distal portion is nested within a concavity of the concave proximal portion, wherein the limacon-shaped distal portion is connected to the concave proximal portion at a proximal location on the limacon-shaped distal portion, and wherein the embolic members and/or material are inserted through the limacon-shaped distal portion into the aneurysm sac.

In this example, embolic members (or material) are inserted into the limacon-shaped (e.g. cardioid or heart shaped) distal portion. In this example, embolic members are coils. In another example, embolic members can be “string of pearl” strands. A “string of pearl” embolic strand can be a series (or sequence) of embolic pieces (e.g. embolic beads, microsponges, or pieces of hydrogel) which are connected by longitudinal strands (e.g. flexible wires, strings, sutures, or springs). One or more “string of pearl” embolic strands can be inserted into the limacon-shaped (e.g. cardioid or heart shaped) distal portion. In another example, embolic members can be separate embolic pieces (e.g. embolic beads, microsponges, or pieces of hydrogel) can be inserted into the limacon-shaped (e.g. cardioid or heart shaped) distal portion. In another example, embolic material can be a liquid or gel which congeals after insertion into an aneurysm sac.

In an example, embolic members (or material) can be inserted through a concave proximal portion into a limacon-shaped (e.g. cardioid or heart shaped) distal portion. In an example, embolic members (or material) can be inserted through an opening in a concave proximal portion into a limacon-shaped distal portion. In an example, embolic members (or material) can be inserted through an opening between the concave proximal portion and the limacon-shaped distal portion into the limacon-shaped distal portion. In an example, embolic members (or material) can be inserted through a concave proximal portion and a limacon-shaped distal portion into the aneurysm sac (e.g. the aneurysm dome).

In an example, insertion of embolic members (or material) into a limacon-shaped (e.g. cardioid or heart shaped) distal portion can expand the limacon-shaped (e.g. cardioid or heart shaped) distal portion. In an example, insertion of embolic members (or material) into a limacon-shaped (e.g. cardioid or heart shaped) distal portion can expand and change the shape of the limacon-shaped (e.g. cardioid or heart shaped) distal portion. In an example, insertion of embolic members (or material) into a limacon-shaped (e.g. cardioid or heart shaped) distal portion can expand the limacon-shaped (e.g. cardioid or heart shaped) distal portion so that it conforms to the walls of even an irregularly-shaped aneurysm sac.

In an example, insertion of embolic members (or material) into a limacon-shaped (e.g. cardioid or heart shaped) distal portion can expand the limacon-shaped (e.g. cardioid or heart shaped) distal portion in a radially-asymmetric manner, enabling it to fill a non-radially-symmetric (e.g. non-spherical) aneurysm sac. In an example, insertion of embolic members (or material) into a limacon-shaped (e.g. cardioid or heart shaped) distal portion can expand the limacon-shaped (e.g. cardioid or heart shaped) distal portion in an irregular manner, enabling it to fill an irregularly shaped (e.g. non-spherical) aneurysm sac. In an example, embolic members (or material) can be inserted into, and contained within, an limacon-shaped (e.g. cardioid or heart shaped) distal portion, thereby expanding the limacon-shaped (e.g. cardioid or heart shaped) distal portion to fill the interior of the aneurysm sac.

In an example, there can be one or more openings in a limacon-shaped (e.g. cardioid or heart shaped) distal portion and a concave proximal portion through which embolic members (or material) are inserted into the limacon-shaped (e.g. cardioid or heart shaped) distal portion. In an example, there can be one or more openings in a limacon-shaped (e.g. cardioid or heart shaped) distal portion and a concave proximal portion along their central longitudinal axes, wherein embolic members (or material) are inserted through these one or more openings into the limacon-shaped (e.g. cardioid or heart shaped) distal portion.

In an example, there can be a first opening in the limacon-shaped (e.g. cardioid or heart shaped) distal portion and a second opening in the concave proximal portion, wherein these first and second openings are aligned to enable insertion of embolic members (or material) through the openings into the limacon-shaped (e.g. cardioid or heart shaped) distal portion. In an example, there can be an opening in a radial constraint which radially-constrains a tubular mesh from which the limacon-shaped (e.g. cardioid or heart shaped) distal portion and the concave proximal portion are formed, wherein embolic members (or material) are inserted through this opening into the limacon-shaped (e.g. cardioid or heart shaped) distal portion.

In an example, there can be a valve in an opening through a concave proximal portion. In an example, this valve can allow embolic members and/or material to enter a limacon-shaped (e.g. cardioid or heart shaped) distal portion when the valve is open and can prevent embolic members and/or material from escaping out of the limacon-shaped distal portion when the valve is closed. In an example, this valve can operate passively. In an example, a valve can be a passive one-way valve which allows embolic members and/or material to enter, but not exit, a limacon-shaped distal portion or a robotic system.

In another example, this valve can be actively opened or closed (remotely) by an operator who is deploying the device. In an example, a valve can be actively opened or closed by a mechanism selected from the group consisting of: application of electrical energy to the valve; pulling or pushing a wire connected to the valve; rotating a wire connected to the valve; pulling a filament or string connected to the valve; activating a small-scale electrical actuator; activating a hydraulic mechanism; activating a pneumatic mechanism; and rotating a catheter.

In an example, a method for deploying an intrasaccular aneurysm occlusion device can comprise: forming a limacon-shaped (e.g. cardioid or heart shaped) distal portion of a device and a concave proximal portion of the device; inserting the limacon-shaped distal portion and the concave proximal portion into an into the aneurysm sac; and inserting embolic members and/or material into the limacon-shaped distal portion.

In an example, a method for deploying an intrasaccular aneurysm occlusion device can comprise: forming a limacon-shaped (e.g. cardioid or heart shaped) distal portion of a device and a concave proximal portion of the device by radially-constraining and inverting a tubular mesh; inserting the limacon-shaped distal portion and the concave proximal portion into an into the aneurysm sac; and inserting embolic members and/or material into the limacon-shaped distal portion.

In an example, a method for deploying an intrasaccular aneurysm occlusion device can comprise: inserting a limacon-shaped (e.g. cardioid or heart shaped) distal portion of a device into an aneurysm sac; inserting a concave proximal portion of the device into the aneurysm sac; drawing the limacon-shaped distal portion and the concave proximal portion closer together in the aneurysm sac; connecting the limacon-shaped distal portion and the concave proximal portion together in the aneurysm sac; and inserting embolic members and/or material into the limacon-shaped distal portion. Example variations discussed elsewhere in this disclosure or priority-linked disclosures can also be applied to this example where relevant.

In an example, a limacon-shaped distal portion of this device can be a flexible sac-filling portion of this device. In an example, a limacon-shaped distal portion of this device can be a distal mesh. In an example, a limacon-shaped distal portion or distal mesh can have a cardioid shape.

In an example, an intrasaccular aneurysm occlusion device can comprise: a distal mesh (or net or porous balloon) which is inserted into an aneurysm sac; wherein the distal mesh is cardioid, crescent shaped, and/or kidney shaped; wherein the distal mesh centroid is a first distance from the aneurysm neck; and a bowl-shaped proximal mesh inserted into the aneurysm sac; wherein the proximal mesh centroid is a second distance from the aneurysm neck; wherein the second distance is less than the first distance; wherein the proximal mesh spans the aneurysm neck; wherein a portion of the distal mesh is within the concavity of the proximal mesh; wherein the proximal mesh is axially-aligned with the distal mesh; and wherein the distal mesh and/or proximal mesh are filled with embolic material (such as microsponges, hydrogels, or coils).

In an example, an intrasaccular aneurysm occlusion device can comprise: a distal mesh which is inserted into an aneurysm sac, wherein the distal mesh is cardioid shaped, and wherein the distal mesh centroid is a first distance from the aneurysm neck; and a bowl-shaped proximal mesh inserted into the aneurysm sac, wherein the proximal mesh centroid is a second distance from the aneurysm neck, wherein the second distance is less than the first distance, wherein a portion of the distal mesh is within a concavity of the proximal mesh, and wherein the distal mesh is filled with embolic material.

In an example, an intrasaccular aneurysm occlusion device can comprise: a distal portion and/or mesh which is inserted into an aneurysm sac, wherein the distal portion and/or mesh is cardioid shaped, and wherein the distal portion and/or mesh centroid is a first distance from the aneurysm neck; and a bowl-shaped proximal portion and/or mesh inserted into the aneurysm sac, wherein the proximal portion and/or mesh centroid is a second distance from the aneurysm neck, wherein the second distance is less than the first distance, wherein a portion of the distal portion and/or mesh is within a concavity of the proximal portion and/or mesh, and wherein the distal portion and/or mesh is filled with embolic material.

In an example, an intrasaccular aneurysm occlusion device can comprise: a limacon and/or cardioid shaped distal portion or mesh which is inserted into an aneurysm sac, wherein the limacon and/or cardioid shaped distal portion or mesh centroid is a first distance from the aneurysm neck; and a bowl-shaped proximal portion or mesh inserted into the aneurysm sac, wherein the bowl-shaped proximal portion or mesh centroid is a second distance from the aneurysm neck, wherein the second distance is less than the first distance, wherein a portion of the limacon and/or cardioid shaped distal portion or mesh is within a concavity of the bowl-shaped proximal portion or mesh, and wherein the limacon and/or cardioid shaped distal portion or mesh is filled with embolic material.

In an example, an intrasaccular aneurysm occlusion device can comprise: a cardioid-shaped distal mesh which is inserted into an aneurysm sac, wherein the cardioid-shaped distal mesh centroid is a first distance from the aneurysm neck; and a bowl-shaped proximal mesh inserted into the aneurysm sac, wherein the bowl-shaped proximal mesh centroid is a second distance from the aneurysm neck, wherein the second distance is less than the first distance, wherein a portion of the cardioid-shaped distal mesh is within a concavity of the bowl-shaped proximal mesh, and wherein the cardioid-shaped distal mesh is filled with embolic material.

In an example, an aneurysm occlusion device can comprise: a distal (wire and/or polymer) mesh inserted into an aneurysm sac; wherein the distal mesh is cardioid shaped; wherein the distal mesh centroid is a first distance from the aneurysm neck; and a proximal mesh inserted into the aneurysm sac; wherein the proximal mesh centroid is a second distance from the aneurysm neck; wherein the second distance is less than the first distance; wherein the proximal mesh spans the aneurysm neck; wherein the proximal mesh is axially-aligned with the distal mesh; and wherein the distal mesh and/or proximal mesh are filled with embolic material (such as microsponges, hydrogels, or coils).

In an example, an aneurysm occlusion device can comprise: a distal mesh (or net or porous balloon) which is inserted into an aneurysm sac; wherein the distal mesh is cardioid shaped; wherein the distal mesh centroid is a first distance from the aneurysm neck; and a bowl-shaped proximal mesh inserted into the aneurysm sac; wherein the proximal mesh centroid is a second distance from the aneurysm neck; wherein the second distance is less than the first distance; wherein the proximal mesh spans the aneurysm neck; wherein a portion of the distal mesh is within the concavity of the proximal mesh; wherein the proximal mesh is axially-aligned with the distal mesh; and wherein the distal mesh and/or proximal mesh are filled with embolic material (such as microsponges, hydrogels, or coils). Relevant example variations discussed elsewhere in this disclosure or priority-linked disclosures can also be applied to the example shown in FIG. 16.

The example shown in FIG. 17 is like the one in FIG. 16 except that the embolic members are “string-of-pearls” embolic strands instead of coils. FIG. 17 shows an example of an intrasaccular aneurysm occlusion device comprising: a limacon-shaped (e.g. cardioid or heart shaped) distal portion 1702 which is inserted into an aneurysm sac; a concave proximal portion 1701 which is inserted into the aneurysm sac and expands to cover the aneurysm neck; and embolic members and/or material 1703 which is inserted into the limacon-shaped distal portion; wherein the limacon-shaped distal portion is between the concave proximal portion and the dome of the aneurysm sac, wherein at least part of the limacon-shaped distal portion is nested within a concavity of the concave proximal portion, and wherein the limacon-shaped distal portion is connected to the concave proximal portion at a proximal location on the limacon-shaped distal portion.

More specifically, the example shown in FIG. 17 can be described as an intrasaccular aneurysm occlusion device comprising: a limacon-shaped (e.g. cardioid or heart shaped) distal portion which is inserted into an aneurysm sac; a bowl-shaped proximal portion which is inserted into the aneurysm sac and expands to cover the aneurysm neck; and “string-of-pearls” embolic strands which are inserted into the limacon-shaped distal portion; wherein the limacon-shaped distal portion is between the bowl-shaped proximal portion and the dome of the aneurysm sac, wherein between 25% and 75% of the limacon-shaped distal portion is nested within a concavity of the bowl-shaped proximal portion, and wherein the limacon-shaped distal portion is connected to the bowl-shaped proximal portion at a proximal location on the limacon-shaped distal portion.

In an example, a string-of-pearls embolic strand can be defined as a series or sequence of embolic members (e.g. beads, polyhedrons, hydrogel pieces, microsponges, or other embolic components) which are connected by flexible longitudinal members (e.g. thin wires, strands, strings, springs, sutures, ribbons, or filaments). In an example, the centers and/or centroids of embolic members (e.g. beads, polyhedrons, hydrogel pieces, microsponges, or other embolic components) in a string-of-pearls embolic strand can be connected by flexible longitudinal members (e.g. thin wires, strands, strings, springs, sutures, ribbons, or filaments). In an example, embolic members (e.g. beads, polyhedrons, hydrogel pieces, microsponges, or other embolic components) in a string-of-pearls embolic strand can be pairwise interconnected by flexible longitudinal members (e.g. thin wires, strands, strings, springs, sutures, ribbons, or filaments).

In an example, there can be variation in the size of embolic pieces along the length of a “string-of-pearls” embolic strand. In an example, embolic pieces which are closer to the distal end of a “string-of-pearls” embolic strand can be larger than embolic pieces which are father from this distal end. In an example, there can be variation in the compressibility and/or durometer of embolic pieces along the length of a “string-of-pearls” embolic strand. In an example, embolic pieces which are closer to the distal end of a “string-of-pearls” embolic strand can be less compressible and/or higher durometer than embolic pieces which are father from this distal end. In an example, there can be variation in the distance between embolic pieces along the length of a “string-of-pearls” embolic strand. In an example, embolic pieces which are closer to the distal end of a “string-of-pearls” embolic strand can be closer together than embolic pieces which are father from this distal end.

In an example, an aneurysm occlusion device can comprise: a distal (wire and/or polymer) mesh inserted into an aneurysm sac; wherein the distal mesh is cardioid shaped; wherein the distal mesh centroid is a first distance from the aneurysm neck; and a proximal mesh inserted into the aneurysm sac; wherein the proximal mesh centroid is a second distance from the aneurysm neck; wherein the second distance is less than the first distance; wherein the proximal mesh spans the aneurysm neck; wherein the proximal mesh is axially-aligned with the distal mesh; and wherein the distal mesh and/or proximal mesh are filled with embolic material (such as microsponges, hydrogels, or coils).

In an example, an aneurysm occlusion device can comprise: a distal mesh (or net or porous balloon) which is inserted into an aneurysm sac; wherein the distal mesh is cardioid shaped; wherein the distal mesh centroid is a first distance from the aneurysm neck; and a bowl-shaped proximal mesh inserted into the aneurysm sac; wherein the proximal mesh centroid is a second distance from the aneurysm neck; wherein the second distance is less than the first distance; wherein the proximal mesh spans the aneurysm neck; wherein a portion of the distal mesh is within the concavity of the proximal mesh; wherein the proximal mesh is axially-aligned with the distal mesh; and wherein the distal mesh and/or proximal mesh are filled with embolic material (such as microsponges, hydrogels, or coils). Example variations discussed elsewhere in this disclosure or priority-linked disclosures can also be applied to this example where relevant.

The example shown in FIG. 18 is like the one in FIG. 16 except that embolic material is liquid or gelatinous material (e.g. a “liquid embolic”) which congeals in the aneurysm sac. FIG. 18 shows an example of an intrasaccular aneurysm occlusion device comprising: a limacon-shaped (e.g. cardioid or heart shaped) distal portion 1802 which is inserted into an aneurysm sac; a concave proximal portion 1801 which is inserted into the aneurysm sac and expands to cover the aneurysm neck; and embolic members and/or material 1803 which is inserted into the limacon-shaped distal portion; wherein the limacon-shaped distal portion is between the concave proximal portion and the dome of the aneurysm sac, wherein at least part of the limacon-shaped distal portion is nested within a concavity of the concave proximal portion, and wherein the limacon-shaped distal portion is connected to the concave proximal portion at a proximal location on the limacon-shaped distal portion.

More specifically, the example shown in FIG. 18 can be described as an intrasaccular aneurysm occlusion device comprising: a limacon-shaped (e.g. cardioid or heart shaped) distal portion which is inserted into an aneurysm sac; a bowl-shaped proximal portion which is inserted into the aneurysm sac and expands to cover the aneurysm neck; and congealing liquid or gelatinous embolic material which is inserted into the limacon-shaped distal portion; wherein the limacon-shaped distal portion is between the bowl-shaped proximal portion and the dome of the aneurysm sac, wherein between 25% and 75% of the limacon-shaped distal portion is nested within a concavity of the bowl-shaped proximal portion, and wherein the limacon-shaped distal portion is connected to the bowl-shaped proximal portion at a proximal location on the limacon-shaped distal portion. Example variations discussed elsewhere in this disclosure or priority-linked disclosures can also be applied to this example where relevant.

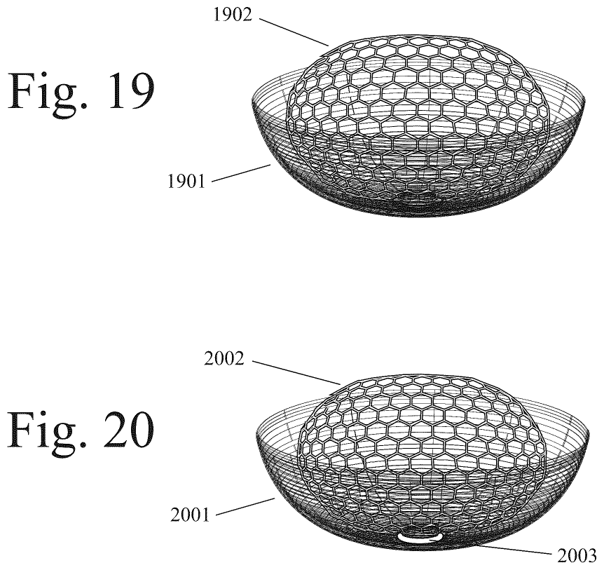

FIGS. 19 through 22 show an example of an intrasaccular aneurysm occlusion device comprising: a flexible distal portion (e.g. a flexible distal net or mesh) which is inserted into an aneurysm sac; and a concave proximal portion (e.g. bowl-shaped proximal portion) which is inserted into the aneurysm sac and expands to cover the aneurysm neck; wherein the flexible distal portion is between the concave proximal portion and the dome of the aneurysm sac, wherein at least part of the flexible distal portion is nested within a concavity of the concave proximal portion, and wherein the flexible distal portion is connected to the concave proximal portion at a proximal location on the flexible distal portion. In a preferred embodiment, the flexible distal portion (e.g. flexible distal net or mesh) is subsequently expanded by being filled with embolic members and/or material.

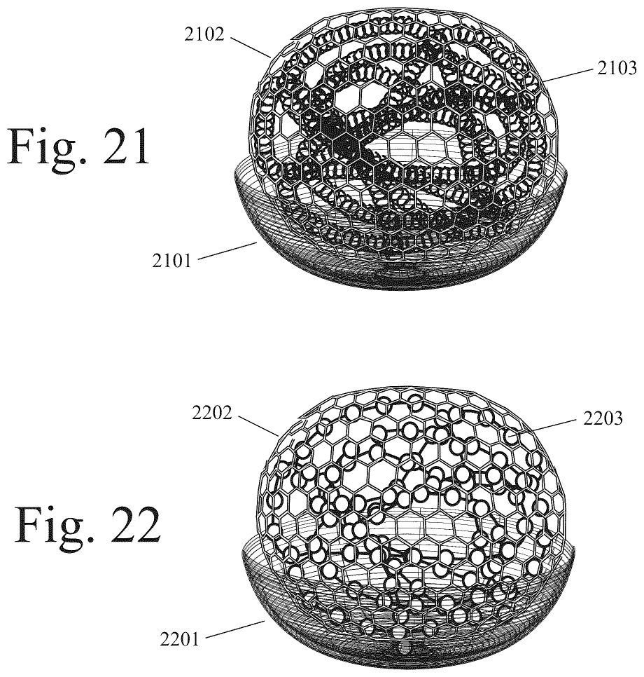

In FIGS. 19 through 22, an intrasaccular aneurysm occlusion device has a flexible distal portion (e.g. a flexible distal net or mesh) and a concave proximal portion (with a bowl shape such as a hemispherical, half-ellipsoidal, half-toroidal, inverted umbrella, or parabolic shape). FIG. 19 shows this device without a radial constraint and before embolic members and/or material have been inserted into the flexible distal portion (e.g. a flexible distal net or mesh). FIG. 20 shows this device with a radial constraint, but before embolic members and/or material have been inserted into the flexible distal portion (e.g. a flexible distal net or mesh). FIG. 21 shows this device after embolic coils have been inserted into the flexible distal portion (e.g. a flexible distal net or mesh). FIG. 22 shows this device after “string-of-pearls” embolic strands have been inserted into the flexible distal portion (e.g. a flexible distal net or mesh).

With respect to specific components, FIG. 19 shows an example of an intrasaccular aneurysm occlusion device comprising: a flexible distal portion (e.g. flexible distal net or mesh) 1902 which is inserted into an aneurysm sac; and a concave proximal portion 1901 which is inserted into the aneurysm sac and expands to cover the aneurysm neck; wherein the flexible distal portion is between the concave proximal portion and the dome of the aneurysm sac, wherein at least part of the flexible distal portion is nested within a concavity of the concave proximal portion, and wherein the flexible distal portion is connected to the concave proximal portion at a proximal location on the flexible distal portion.

The example shown in FIG. 19 can also be described as an intrasaccular aneurysm occlusion device comprising: a flexible distal net or mesh which is inserted into an aneurysm sac; and a bowl-shaped proximal portion which is inserted into the aneurysm sac and expands to cover the aneurysm neck; wherein the distal net or mesh is between the bowl-shaped proximal portion and the dome of the aneurysm sac, wherein at least part of the distal net or mesh is nested within a concavity of the bowl-shaped proximal portion, and wherein the distal net or mesh is connected to the bowl-shaped proximal portion at a proximal location on the distal net or mesh.

In an example, a flexible distal portion can be a distal net or mesh. In an example, a flexible distal portion can be a polymer net or mesh. In an example, a flexible distal portion can be woven or braided from polymer strands, filaments, or threads. In an example, a flexible distal portion can be 3D printed. In an example, a flexible distal portion can be 3D printed using a flow of liquid polymer material.

In an example, a flexible distal portion can have a first configuration before it is expanded by the insertion of embolic members and/or material and a second configuration after it has been expanded by the insertion of embolic members and/or material, wherein the second configuration is at least 50% larger (e.g. has at least 50% greater interior volume) than the first configuration. In an example, a flexible distal portion can have a first configuration before it is expanded by the insertion of embolic members and/or material and a second configuration after it has been expanded by the insertion of embolic members and/or material, wherein the flexible distal portion is folded and/or pleated in the first configuration and unfolded and/or unpleated in the second configuration.

In an example, a flexible distal portion (e.g. net or mesh) can be pushed or pulled from a catheter into an aneurysm sac by a wire. In an example, a flexible distal portion (e.g. net or mesh) can be pushed or pulled from a catheter into an aneurysm sac by a wire, wherein the wire is removably connected to the distal surface of the flexible distal portion (e.g. net or mesh). In an example, a flexible distal portion (e.g. net or mesh) can be pushed or pulled out of a catheter into an aneurysm sac by a wire and then further expanded by insertion of embolic members and/or material.

In an example, a flexible distal portion can be expanded by the insertion of embolic members and/or material in it, so that it conforms to the contours of the walls of even an irregularly-shaped aneurysm sac. In an example, a flexible distal portion (e.g. net or mesh) can have a radially-symmetric first configuration when it is first inserted into an aneurysm sac and then be expanded into a radially-asymmetric configuration by being filled with embolic members and/or material. In an example, a flexible distal portion (e.g. net or mesh) which is inserted into an non-globular (e.g. lobed) aneurysm sac can have a generally globular first configuration when it is first inserted into an aneurysm sac and then be expanded into a non-globular (e.g. lobed) second configuration which conforms to the walls of the aneurysm sac by the insertion of embolic members and/or material.

In an example, a flexible distal portion (e.g. net or mesh) which is inserted into an non-globular (e.g. lobed) aneurysm sac can have a generally globular first configuration when it is first inserted into an aneurysm sac and then be expanded into a non-globular (e.g. lobed) second configuration which conforms to the walls of the aneurysm sac by being filled with embolic members and/or material. In an example, a flexible distal portion (e.g. net or mesh) which is inserted into an non-globular (e.g. multi-lobed) aneurysm sac can have a generally globular first configuration when it is first inserted into an aneurysm sac and then be expanded into a non-globular (e.g. lobed) second configuration which conforms to the walls of the aneurysm sac by being filled with embolic members and/or material.

In an example, a flexible distal portion can have a first configuration before it is expanded by the insertion of embolic members and/or material and a second configuration after it is expanded by the insertion of embolic members and/or material, wherein more than 75% of the flexible distal portion is nested in a concavity of a concave proximal portion in the first configuration and wherein less than 75% of the flexible portion is nested in the concavity of the concave proximal portion in the second configuration.

In an example, a flexible distal portion (e.g. net or mesh) can have a first configuration before being filled with embolic members and/or material and a second configuration after having been filled with embolic members and/or material, wherein the first configuration has a width which is less than the width of a concave proximal portion and the second configuration has a width which is greater than the width of the concave proximal portion. In an example, a flexible distal portion (e.g. net or mesh) can have a first configuration before being filled with embolic members and/or material and a second configuration after having been filled with embolic members and/or material, wherein a central axis of the flexible distal portion is aligned with a central axis of a concave proximal portion in the first configuration, but not in the second configuration.

In an example, a flexible distal portion can have a first configuration before it is expanded by the insertion of embolic members and/or material and a second configuration after it is expanded by the insertion of embolic members and/or material, wherein more than half of the flexible distal portion is nested in a concavity of a concave proximal portion in the first configuration and wherein less than half of the flexible portion is nested in the concavity of the concave proximal portion in the second configuration.

In an example, there can be openings (e.g. holes) in a flexible distal portion (e.g. net or mesh) of an intrasaccular aneurysm occlusion device. In an example, these openings can be sized to allow blood to escape from the aneurysm sac as the device is being expanded in the sac, but not allow embolic members and/or material to escape from the flexible distal portion. In an example, these openings can be sized to allow blood to escape from the aneurysm sac as the device is being expanded in the sac, but contain embolic members and/or material in the flexible distal portion.

In an example, openings (e.g. holes) in the flexible distal portion (e.g. net or mesh) can be smaller than embolic members (e.g. pieces) which are inserted into the flexible distal portion so that these embolic members are contained in the flexible distal portion. In an example, openings (e.g. holes) in the flexible distal portion (e.g. net or mesh) can be larger than embolic pieces which are strung together in “string-of-pearls” strands, but the strands are still contained within the flexible distal portion due to the wires, strings, or other filaments which connect the embolic pieces together.

In an example, openings (e.g. holes) in the distal half of a flexible distal portion (e.g. net or mesh) can be larger than openings (e.g. holes) in the proximal half of the flexible distal portion (e.g. net or mesh). In an example, openings in the distal half of a flexible distal portion (e.g. net or mesh) can be larger than embolic members (e.g. pieces), but openings in the proximal half of the flexible distal portion (e.g. net or mesh) can be smaller than embolic members (e.g. pieces).

In an example, a flexible distal portion can have a first configuration before it is expanded by the insertion of embolic members and/or material and a second configuration after it is expanded by the insertion of embolic members and/or material, wherein between 66% and 100% of the flexible distal portion is nested in a concavity of a concave proximal portion in the first configuration and wherein between 33% and 66% of the flexible portion is nested in the concavity of the concave proximal portion in the second configuration.

In an example, a flexible distal portion (e.g. net or mesh) can be made by weaving or braiding polymer strands, filaments, or threads together. In an example, a flexible distal portion (e.g. net or mesh) can be made with a combination of metal wires and polymer strands, filaments, or threads. In an example, a flexible distal portion (e.g. net or mesh) can be by weaving or braiding together a combination of metal wires and polymer strands, filaments, or threads. In an example, a flexible distal portion can be made by using a laser to create holes in a balloon.

In an example, a flexible distal portion (e.g. net or mesh) can have a first configuration before it is filled with embolic members and/or material and a second configuration after it has been filled with embolic members and/or material, wherein the flexible distal portion stretches to at least twice its size as it changes from its first configuration to its second configuration. In an example, a flexible distal portion (e.g. net or mesh) can have a first configuration before it is filled with embolic members and/or material and a second configuration after it has been filled with embolic members and/or material, wherein the flexible distal portion is expanded in a radially-asymmetric manner as it changes from its first configuration to its second configuration.

In an example, openings in a flexible distal portion (e.g. net or mesh) can be smaller than embolic members (e.g. beads, balls, microsponges, or hydrogel pieces) which are inserted into the flexible distal portion. In an example, openings (e.g. holes) in a distal part of a flexible distal portion (e.g. net or mesh) can be larger than openings (e.g. holes) in a proximal part of the flexible distal portion (e.g. net or mesh).

In an example, a flexible distal portion (e.g. net or mesh) can have a central opening through which embolic members are inserted, wherein the width of the central opening is larger than the width of the embolic members, and a plurality of other openings (e.g. holes) whose widths are smaller than the width of the embolic members, so that embolic members do not escape through the other openings. In an example, a flexible distal portion (e.g. net or mesh) can comprise: a central large opening through which embolic members are inserted into the flexible distal portion, wherein the width of the central opening is larger than the widths of the embolic members, and wherein a valve closes the large opening after embolic members have been inserted; and a plurality of smaller openings (e.g. holes) whose widths are smaller than the widths of the embolic members so that embolic members do not escape through the smaller openings.

In an example, a flexible distal portion can be a honeycomb mesh with a plurality of hexagonal openings. In an example, a flexible distal portion can be an elastic honeycomb mesh with a plurality of hexagonal openings. In an example, a flexible distal portion can be an elastic polymer honeycomb mesh with a plurality of hexagonal openings. In an example, a flexible distal portion can be a woven mesh with a plurality of quadrilateral openings. In an example, a flexible distal portion can be an elastic woven mesh with a plurality of quadrilateral openings. In an example, a flexible distal portion can be an elastic polymer woven mesh with a plurality of quadrilateral openings.

In an example, a flexible distal portion can be woven or braided from polymer strands, filaments, or threads. In an example, a flexible distal portion can be a polymer net or mesh. In an example, a flexible distal portion can be an elastic polymer net or mesh. In an example, openings (e.g. holes) in a flexible distal net or mesh can be of uniform size. In an example, a flexible distal portion (e.g. net or mesh) can unfold or uncurl to at least twice its size when first inserted into an aneurysm sac. In an example, openings (e.g. holes) in the proximal half of a flexible distal portion (e.g. net or mesh) can be larger than openings (e.g. holes) in the distal half of the flexible distal portion (e.g. net or mesh).

In an example, there can be a central opening (e.g. hole) in the proximal surface of a flexible distal portion (e.g. net or mesh) through which embolic members and/or material are inserted into the interior of the flexible distal portion. In an example, a device can further comprise valve which opens or closes this central opening. In an example, this valve can be remotely opened or closed by a device operator. In an example, this valve can be remotely opened or closed by a device operator by pulling or pushing a wire. In an example, this valve can be remotely opened or closed by a device operator by rotating a wire in a clockwise or counter clock-wise direction. In an example, this valve can be remotely opened or closed by the transmission of electrical energy to the valve.

In an example, a flexible distal portion (e.g. distal net or mesh) of a device can have a generally globular first configuration before it is expanded by the insertion of embolic members and/or embolic material and a non-globular second configuration after it has been filled with embolic members and/or embolic material. In an example, a flexible distal portion can have a generally globular first configuration before it is expanded by the insertion of embolic members and/or embolic material and a radially-asymmetric non-globular second configuration after it has been filled with embolic members and/or embolic material. In an example, a flexible distal portion can have a generally globular first configuration before it is expanded by the insertion of embolic members and/or embolic material and a multi-lobed non-globular second configuration after it has been filled with embolic members and/or embolic material.

In an example, a flexible distal portion (e.g. distal net or mesh) can be a polymer net or mesh which is made from an elastomeric polymer. In an example, a flexible distal portion (e.g. distal net or mesh) can be made from polydimethylsiloxane (PDMS) or other silicone-based material. In an example, a flexible distal portion (e.g. distal net or mesh) can be made with polyolefin elastomer. In an example, a flexible distal portion (e.g. distal net or mesh) can be made with polyurethane.

In an example, a flexible distal portion (e.g. distal net or mesh) can be made from thermoplastic polyurethane (TPU). In an example, a flexible distal portion (e.g. distal net or mesh) can be made with hydrogel. In an example, a flexible distal portion (e.g. distal net or mesh) can be made with polyester. In an example, a flexible distal portion (e.g. distal net or mesh) can be made with polyglycolic acid (PGA). In an example, a flexible distal portion (e.g. distal net or mesh) can be made with polylactic acid (PLA). In an example, a flexible distal portion can be woven or braided from metal wires. In an example, a flexible distal portion can be woven or braided from nitinol wires.

In an example, a flexible distal portion can have a first configuration before it is expanded by the insertion of embolic members and/or material and a second configuration after it is expanded by the insertion of embolic members and/or material, wherein more than two-thirds of the flexible distal portion is nested in a concavity of a concave proximal portion in the first configuration and wherein less than one-third of the flexible portion is nested in the concavity of the concave proximal portion in the second configuration.

In an example, a flexible distal portion (e.g. net or mesh) of a device is more flexible than a concave proximal portion of the device. In an example, the shape of a flexible distal portion (e.g. net or mesh) changes when embolic members and/or material are inserted into it. In an example, a flexible distal portion (e.g. net or mesh) can conform to the irregular shape of the walls of an irregularly-shaped aneurysm sac after insertion of embolic members and/or material into the flexible distal portion (e.g. net or mesh).

In this example, the concave proximal portion has a bowl shape. In various examples, a concave proximal portion can have a shape which is selected from the group consisting of: hemispherical, half-ellipsoidal, half-toroidal (e.g. lower half), inverted umbrella shape, inverted dome shape, parabolic, and conic section. In an example, a concave proximal portion can have a radially-compressed configuration as it is delivered through a catheter into an aneurysm sac. In an example, a concave proximal portion can self-expand radially after it has been inserted into the aneurysm sac. In an example, a concave proximal portion can be expanded radially after it has been inserted into the aneurysm sac by moving a wire to which it is attached or by the application of electrical energy.

In an example, a distal end of a flexible distal portion (e.g. net or mesh) can be radially-constrained (e.g. pinched). In an example, a distal end of a flexible distal portion (e.g. net or mesh) can be radially-constrained (e.g. pinched) closed by an annular ring, band, wire, string, or cylinder. In an example, the distal end of a flexible distal portion (e.g. net or mesh) may be closed (e.g. pinched and/or radially-constrained) by a radial constraint (e.g. an annular ring, band, or wire).

In an example, a radial constraint can be a compound component with an outer part and an inner part. In an example, the outer and inner parts can be nested, coaxial, and/or concentric. In an example, a radial-constraint can include an outer part (on the outside of the tubular member) and an inner part (on the inside of the tubular member), wherein the tubular member is held between (e.g. passes between) the outer part and the inner part. In an example, there can be an opening through the inner part through which embolic members and/or material is inserted into the flexible distal portion (e.g. net or mesh).

In an example, a radial-constraint can include two nested (e.g. coaxial or concentric) bands, rings, or cylinders comprising: an outer band, ring, or cylinder (on the outside of the tubular member) and an inner band, ring, or cylinder (on the inside of the tubular member), wherein the tubular member is held between (e.g. passes through) the outer band, ring, or cylinder and the inner band, ring, or cylinder, and wherein embolic members (or material) are inserted in the flexible distal portion (e.g. net or mesh) through an opening (e.g. lumen) the inner band, ring, or cylinder.

In an example, a flexible distal portion (e.g. net or mesh) and a concave proximal portion can be made separately and then connected together. In another example, a flexible distal portion (e.g. net or mesh) and a concave proximal portion can be made from different materials (and/or with different processes) and then connected together. In another example, a flexible distal portion (e.g. net or mesh) and a concave proximal portion can be made from different materials (and/or with different processes) and then connected together before they are inserted into a catheter for deliver into an aneurysm sac. In another example, a flexible distal portion (e.g. net or mesh) and a concave proximal portion can be made from different materials (and/or with different processes) and then connected after they have been inserted into an aneurysm sac.

In an example, a concave proximal portion can be less elastic (e.g. made with less elastic material) than a flexible distal portion (e.g. net or mesh). In an example, the concave proximal portion can be thicker (e.g. made with thicker wires) than the flexible distal portion (e.g. net or mesh). In an example, the concave proximal portion can be less compressible (e.g. made with higher durometer material) than the flexible distal portion (e.g. net or mesh). In an example, the concave proximal portion can be denser (e.g. made with a denser braid or weave) than the flexible distal portion (e.g. net or mesh). In an example, the concave proximal portion can be less porous (e.g. made with a liquid-impermeable layer) than the flexible distal portion (e.g. net or mesh). In an example, the concave proximal portion can have more layers (e.g. two or three layers) than the flexible distal portion (e.g. net or mesh). In an example, the concave proximal portion can comprise a liquid-impermeable (e.g. polymer membrane) layer between two wire layers.

In an example, a flexible distal portion (e.g. net or mesh) can be more elastic (e.g. made with more elastic material) than a concave proximal portion. In an example, the flexible distal portion (e.g. net or mesh) can be thinner (e.g. made with thinner wires) than the concave proximal portion. In an example, the flexible distal portion (e.g. net or mesh) can be more compressible (e.g. made with lower durometer material) than the concave proximal portion. In an example, the flexible distal portion (e.g. net or mesh) can be less dense (e.g. made with a less dense braid or weave) than the concave proximal portion. In an example, the flexible distal portion (e.g. net or mesh) can be more porous (e.g. made with larger holes) than the concave proximal portion. In an example, the flexible distal portion (e.g. net or mesh) can have fewer layers than the concave proximal portion.

In an example, a flexible distal portion (e.g. net or mesh) and a concave proximal portion can both be made from wire. In an example, a flexible distal portion (e.g. net or mesh) and a concave proximal portion can both be made by braiding or weaving wires. In an example, a flexible distal portion (e.g. net or mesh) and a concave proximal portion can be made by braiding or weaving nitinol wires. In another example, a flexible distal portion (e.g. net or mesh) and a concave proximal portion can be made by 3D printing. In an example, a flexible distal portion (e.g. net or mesh) can be made from a polymer and a concave proximal portion can be made from metal. In an example, a flexible distal portion can be a polymer mesh or net and a concave proximal portion can be a braid or weave of metal wires. In an example, a flexible distal portion can be a flexible polymer net or mesh and a concave proximal portion can be a wire mesh or stent.

In an example, a flexible distal portion (e.g. net or mesh) and a concave proximal portion can be attached to each other before they are delivered through a catheter into an aneurysm sac. In an example, a flexible distal portion (e.g. net or mesh) and a concave proximal portion can be inserted into an aneurysm sac at substantially the same time. In an example, a flexible distal portion (e.g. net or mesh) and a concave proximal portion can be nested (e.g. overlap) before and after they are inserted into an aneurysm. In another example, a flexible distal portion (e.g. net or mesh) and a concave proximal portion may not be nested (e.g. not overlap) while they are being delivered through a catheter into an aneurysm sac, but are connected to each other within the aneurysm sac so that they become nested (e.g. overlapping) in the aneurysm sac.

In an example, a flexible distal portion (e.g. net or mesh) and a concave proximal portion can be delivered through a catheter sequentially so that they do not overlap while they are being delivered through the catheter. In an example, a flexible distal portion (e.g. net or mesh) and a concave proximal portion can be separated by a longitudinal distance as they are being delivered through a catheter sequentially, but are moved closer to each other (and connected) after they have been inserted into an aneurysm sac.

In an example, a flexible distal portion (e.g. net or mesh) and a concave proximal portion are not nested as they are delivered sequentially through a catheter into an aneurysm sac, but are nested after they are connected to each other within the aneurysm sac. In an example, a flexible distal portion (e.g. net or mesh) and a concave proximal portion do not overlap as they are delivered sequentially through a catheter into an aneurysm sac, but do overlap after they are connected to each other within the aneurysm sac.

In an example, a method for deploying an intrasaccular aneurysm occlusion device can comprise: inserting a flexible distal portion (e.g. distal net or mesh) into an aneurysm sac; inserting a concave proximal portion (e.g. bowl-shaped proximal portion) of the device into the aneurysm sac; drawing the flexible distal portion and the concave proximal portion closer together in the aneurysm sac; and then connecting the flexible distal portion and the concave proximal portion together in the aneurysm sac.

In an example, a flexible distal portion (e.g. distal net or mesh) and a concave proximal portion (e.g. bowl-shaped proximal portion) can be separated by a longitudinal distance as they are being delivered through a catheter sequentially, but are moved closer to each other (and connected) by movement of a wire (connected to one or both of them) after they have been inserted into an aneurysm sac. In an example, this device can further comprise a wire which is attached to the flexible distal portion (e.g. net or mesh). In an example, this device can further comprise a wire which is attached to the distal end of the flexible distal portion (e.g. net or mesh), wherein the flexible distal portion is drawn closer to the concave proximal portion when the wire is pulled.

In an example, a device can further comprise a wire which is attached to a concave proximal portion. In an example, a device can further comprise a wire which is attached to a concave proximal portion, wherein the concave proximal portion is drawn closer to the flexible distal portion (e.g. net or mesh) when the wire is pushed. In an example, ta device can further comprise two wires, one wire which is attached to a flexible distal portion (e.g. net or mesh) and one wire which is attached to a concave proximal portion, wherein the pulling or pushing one or both wires moves the two portions closer together.

In an example, a flexible distal portion (e.g. net or mesh) and a concave proximal portion (e.g. bowl-shaped proximal portion) can be separated by a longitudinal distance as they are being delivered through a catheter sequentially, but can be moved closer to each other (and connected) by application of electricity (to one or both of them) after they have been inserted into an aneurysm sac. Relevant example variations discussed elsewhere in this disclosure or priority-linked disclosures can also be applied to the example shown in FIG. 19.

The example shown in FIG. 20 is like the one in FIG. 19 except that it includes a radial constraint (e.g. an annular ring, band, cylinder, or wire) around the connection between the flexible distal portion (e.g. net or mesh) and the concave proximal portion (e.g. bowl-shaped proximal portion). FIG. 20 shows an intrasaccular aneurysm occlusion device comprising: a flexible distal portion (e.g. net or mesh) 2002 which is inserted into an aneurysm sac; a concave proximal portion (e.g. bowl-shaped proximal portion) 2001 which is inserted into the aneurysm sac and expands to cover the aneurysm neck; and an annular constraint 2003 which radially constrains the connection between the flexible distal portion and the concave proximal portion; wherein the flexible distal portion is between the concave proximal portion and the dome of the aneurysm sac, wherein at least part of the flexible distal portion is nested within a concavity of the concave proximal portion, and wherein the flexible distal portion is connected to the concave proximal portion at a proximal location on the flexible distal portion.

More specifically, the example shown in FIG. 20 can be described as an intrasaccular aneurysm occlusion device comprising: a flexible distal net or mesh which is inserted into an aneurysm sac; a bowl-shaped proximal portion which is inserted into the aneurysm sac and expands to cover the aneurysm neck; and a radial constraint around the connection between the flexible distal net or mesh and the bowl-shaped proximal portion; wherein the flexible distal net or mesh is between the bowl-shaped proximal portion and the dome of the aneurysm sac, wherein between 25% and 75% of the flexible distal net or mesh is nested within a concavity of the bowl-shaped proximal portion, and wherein the flexible distal net or mesh is connected to the bowl-shaped proximal portion at a proximal location on the flexible distal net or mesh. Example variations discussed elsewhere in this disclosure or priority-linked disclosures can also be applied to this example where relevant.

The example shown in FIG. 21 is like the one in FIG. 19 except that embolic members and/or material have been inserted into the flexible distal portion (e.g. net or mesh). In this example, the embolic members are coils. FIG. 21 shows an example of an intrasaccular aneurysm occlusion device comprising: a flexible distal portion (e.g. net or mesh) 2102 which is inserted into an aneurysm sac; a concave proximal portion (e.g. bowl-shaped proximal portion) 2101 which is inserted into the aneurysm sac and expands to cover the aneurysm neck; and embolic members and/or material 2103 which are inserted into the flexible distal portion; wherein the flexible distal portion is between the concave proximal portion and the dome of the aneurysm sac, wherein at least part of the flexible distal portion is nested within a concavity of the concave proximal portion, and wherein the flexible distal portion is connected to the concave proximal portion at a proximal location on the flexible distal portion.

More specifically, the example shown in FIG. 21 can be described as an intrasaccular aneurysm occlusion device comprising: a flexible distal net or mesh which is inserted into an aneurysm sac; a bowl-shaped proximal portion which is inserted into the aneurysm sac and expands to cover the aneurysm neck; and embolic coils which are inserted into the flexible distal net or mesh; wherein the flexible distal net or mesh) is between the bowl-shaped proximal portion and the dome of the aneurysm sac, wherein between 25% and 75% of the flexible distal net or mesh is nested within a concavity of the bowl-shaped proximal portion, and wherein the flexible distal net or mesh is connected to the bowl-shaped proximal portion at a proximal location on the flexible distal net or mesh.

In an example, an intrasaccular aneurysm occlusion device can comprise: a flexible distal portion (e.g. net or mesh) which is inserted into an aneurysm sac; a concave proximal portion (e.g. bowl-shaped proximal portion) which is inserted into the aneurysm sac and expands to cover the aneurysm neck; and embolic members and/or material; wherein the flexible distal portion is between the concave proximal portion and the dome of the aneurysm sac, wherein at least part of the flexible distal portion is nested within a concavity of the concave proximal portion, wherein the flexible distal portion is connected to the concave proximal portion at a proximal location on the flexible distal portion, and wherein the embolic members and/or material are inserted through the concave proximal portion into the flexible distal portion.

In an example, an intrasaccular aneurysm occlusion device can comprise: a flexible distal portion (e.g. net or mesh) which is inserted into an aneurysm sac; a concave proximal portion (e.g. bowl-shaped proximal portion) which is inserted into the aneurysm sac and expands to cover the aneurysm neck; and embolic members and/or material; wherein the flexible distal portion is between the concave proximal portion and the dome of the aneurysm sac, wherein at least part of the flexible distal portion is nested within a concavity of the concave proximal portion, wherein the flexible distal portion is connected to the concave proximal portion at a proximal location on the flexible distal portion, and wherein the embolic members and/or material are inserted through an opening in concave proximal portion into the flexible distal portion.

In another example, an intrasaccular aneurysm occlusion device can comprise: a flexible distal portion (e.g. net or mesh) which is inserted into an aneurysm sac; a concave proximal portion (e.g. bowl-shaped proximal portion) which is inserted into the aneurysm sac and expands to cover the aneurysm neck; and embolic members and/or material; wherein the flexible distal portion is between the concave proximal portion and the dome of the aneurysm sac, wherein at least part of the flexible distal portion is nested within a concavity of the concave proximal portion, wherein the flexible distal portion is connected to the concave proximal portion at a proximal location on the flexible distal portion, and wherein the embolic members and/or material are inserted through the flexible distal portion into the aneurysm sac.

In this example, embolic members (or material) are inserted into a flexible distal portion (e.g. net or mesh). In this example, embolic members are coils. In another example, embolic members can be “string of pearl” strands. A “string of pearl” embolic strand can be a series (or sequence) of embolic pieces (e.g. embolic beads, microsponges, or pieces of hydrogel) which are connected by longitudinal strands (e.g. flexible wires, strings, sutures, or springs). One or more “string of pearl” embolic strands can be inserted into a flexible distal portion (e.g. net or mesh). In another example, embolic members can be separate embolic pieces (e.g. embolic beads, microsponges, or pieces of hydrogel) can be inserted into a flexible distal portion (e.g. net or mesh). In another example, embolic material can be a liquid or gel which congeals after insertion into an aneurysm sac.

In an example, embolic members (or material) can be inserted through a concave proximal portion (e.g. bowl-shaped proximal portion) into a flexible distal portion (e.g. net or mesh). In an example, embolic members (or material) can be inserted through an opening in a concave proximal portion (e.g. bowl-shaped proximal portion) into a flexible distal portion (e.g. net or mesh). In an example, embolic members (or material) can be inserted through an opening between the concave proximal portion (e.g. bowl-shaped proximal portion) and the flexible distal portion (e.g. net or mesh) into the flexible distal portion (e.g. net or mesh). In an example, embolic members (or material) can be inserted through a concave proximal portion (e.g. bowl-shaped proximal portion) and a flexible distal portion (e.g. net or mesh) into the aneurysm sac (e.g. the aneurysm dome).

In an example, insertion of embolic members (or material) into a flexible distal portion (e.g. net or mesh) can expand a flexible distal portion. In an example, insertion of embolic members (or material) into a flexible distal portion can expand and change the shape of the flexible distal portion. In an example, insertion of embolic members (or material) into a flexible distal portion can expand the flexible distal portion so that it conforms to the walls of even an irregularly-shaped aneurysm sac. In an example, insertion of embolic members (or material) into a flexible distal portion can expand the flexible distal portion in a radially-asymmetric manner, enabling it to fill a non-radially-symmetric (e.g. non-spherical) aneurysm sac. In an example, insertion of embolic members (or material) into a flexible distal portion can expand the flexible distal portion in an irregular manner, enabling it to fill an irregularly shaped (e.g. non-spherical) aneurysm sac. In an example, embolic members (or material) can be inserted into, and contained within, an flexible distal portion, thereby expanding the flexible distal portion to fill the interior of the aneurysm sac.

In an example, there can be one or more openings in a flexible distal portion (e.g. net or mesh) and a concave proximal portion (e.g. bowl-shaped proximal portion) through which embolic members (or material) are inserted into the flexible distal portion. In an example, there can be one or more openings in a flexible distal portion and a concave proximal portion along their central longitudinal axes, wherein embolic members (or material) are inserted through these one or more openings into the flexible distal portion. In an example, there can be a first opening in the flexible distal portion and a second opening in the concave proximal portion, wherein these first and second openings are aligned to enable insertion of embolic members (or material) through the openings into the flexible distal portion. In an example, there can be an opening in a radial constraint which radially-constrains a tubular mesh from which the flexible distal portion and the concave proximal portion are formed, wherein embolic members (or material) are inserted through this opening into the flexible distal portion.

In an example, there can be a valve in an opening through a concave proximal portion (e.g. bowl-shaped proximal portion). In an example, this valve can allow embolic members and/or material to enter a flexible distal portion (e.g. net or mesh) when the valve is open and can prevent embolic members and/or material from escaping out of the flexible distal portion when the valve is closed. In an example, this valve can operate passively. In an example, a valve can be a passive one-way valve which allows embolic members and/or material to enter, but not exit, a flexible distal portion or a robotic system. In another example, this valve can be actively opened or closed (remotely) by an operator who is deploying the device. In an example, a valve can be actively opened or closed by a mechanism selected from the group consisting of: application of electrical energy to the valve; pulling or pushing a wire connected to the valve; rotating a wire connected to the valve; pulling a filament or string connected to the valve; activating a small-scale electrical actuator; activating a hydraulic mechanism; activating a pneumatic mechanism; and rotating a catheter.

In an example, a method for deploying an intrasaccular aneurysm occlusion device can comprise: forming a flexible distal portion (e.g. net or mesh) of a device and a concave proximal portion (e.g. bowl-shaped proximal portion) of the device; inserting the flexible distal portion and the concave proximal portion into an into the aneurysm sac; and inserting embolic members and/or material into the flexible distal portion. In an example, a method for deploying an intrasaccular aneurysm occlusion device can comprise: forming a flexible distal portion (e.g. net or mesh) of a device and a concave proximal portion (e.g. bowl-shaped proximal portion) of the device by radially-constraining and inverting a tubular mesh; inserting the flexible distal portion and the concave proximal portion into an into the aneurysm sac; and inserting embolic members and/or material into the flexible distal portion.

In an example, a method for deploying an intrasaccular aneurysm occlusion device can comprise: inserting a flexible distal portion (e.g. net or mesh) of a device into an aneurysm sac; inserting a concave proximal portion (e.g. bowl-shaped proximal portion) of the device into the aneurysm sac; drawing the flexible distal portion and the concave proximal portion closer together in the aneurysm sac; connecting the flexible distal portion and the concave proximal portion together in the aneurysm sac; and inserting embolic members and/or material into the flexible distal portion. Example variations discussed elsewhere in this disclosure or priority-linked disclosures can also be applied to this example where relevant.

The example shown in FIG. 22 is like the one in FIG. 21 except that the embolic members are “string-of-pearls” embolic strands instead of coils. FIG. 22 shows an example of an intrasaccular aneurysm occlusion device comprising: a flexible distal portion (e.g. net or mesh) 2202 which is inserted into an aneurysm sac; a concave proximal portion (e.g. bowl-shaped proximal portion) 2201 which is inserted into the aneurysm sac and expands to cover the aneurysm neck; and embolic members and/or material 2203 which is inserted into the flexible distal portion; wherein the flexible distal portion is between the concave proximal portion and the dome of the aneurysm sac, wherein at least part of the flexible distal portion is nested within a concavity of the concave proximal portion, and wherein the flexible distal portion is connected to the concave proximal portion at a proximal location on the flexible distal portion.

More specifically, the example shown in FIG. 22 can be described as an intrasaccular aneurysm occlusion device comprising: a flexible distal net or mesh which is inserted into an aneurysm sac; a bowl-shaped proximal portion which is inserted into the aneurysm sac and expands to cover the aneurysm neck; and “string-of-pearls” embolic strands which are inserted into the flexible distal net or mesh; wherein the flexible distal net or mesh is between the bowl-shaped proximal portion and the dome of the aneurysm sac, wherein between 25% and 75% of the flexible distal net or mesh is nested within a concavity of the bowl-shaped proximal portion, and wherein the flexible distal net or mesh is connected to the bowl-shaped proximal portion at a proximal location on the flexible distal net or mesh.

In an example, a string-of-pearls embolic strand can be defined as a series or sequence of embolic members (e.g. beads, polyhedrons, hydrogel pieces, microsponges, or other embolic components) which are connected by flexible longitudinal members (e.g. thin wires, strands, strings, springs, sutures, ribbons, or filaments). In an example, the centers and/or centroids of embolic members (e.g. beads, polyhedrons, hydrogel pieces, microsponges, or other embolic components) in a string-of-pearls embolic strand can be connected by flexible longitudinal members (e.g. thin wires, strands, strings, springs, sutures, ribbons, or filaments). In an example, embolic members (e.g. beads, polyhedrons, hydrogel pieces, microsponges, or other embolic components) in a string-of-pearls embolic strand can be pairwise interconnected by flexible longitudinal members (e.g. thin wires, strands, strings, springs, sutures, ribbons, or filaments).

In an example, there can be variation in the size of embolic pieces along the length of a “string-of-pearls” embolic strand. In an example, embolic pieces which are closer to the distal end of a “string-of-pearls” embolic strand can be larger than embolic pieces which are father from this distal end. In an example, there can be variation in the compressibility and/or durometer of embolic pieces along the length of a “string-of-pearls” embolic strand. In an example, embolic pieces which are closer to the distal end of a “string-of-pearls” embolic strand can be less compressible and/or higher durometer than embolic pieces which are father from this distal end. In an example, there can be variation in the distance between embolic pieces along the length of a “string-of-pearls” embolic strand. In an example, embolic pieces which are closer to the distal end of a “string-of-pearls” embolic strand can be closer together than embolic pieces which are father from this distal end. Example variations discussed elsewhere in this disclosure or priority-linked disclosures can also be applied to this example where relevant.

I claim:

1. An intrasaccular aneurysm occlusion device comprising:
a convex distal portion which is inserted into an aneurysm sac; and
a concave proximal portion which is inserted into the aneurysm sac and expands to cover the aneurysm neck;
wherein the convex distal portion is between the concave proximal portion and the dome of the aneurysm sac, wherein at least part of the convex distal portion is nested within a concavity of the concave proximal portion, and wherein the convex distal portion is connected to the concave proximal portion at a proximal location on the convex distal portion.

2. The device in claim 1 wherein the convex distal portion has a globular shape.

3. The device in claim 2 wherein the concave proximal portion has a bowl shape.

4. The device in claim 3 wherein embolic coils are inserted into the convex distal portion.

5. The device in claim 3 wherein string-of-pearls embolic strands are inserted into the convex distal portion.

6. The device in claim 5 wherein a string-of-pearls embolic strand is a series or sequence of embolic members which are connected by flexible longitudinal members.

7. The device in claim 6 wherein embolic members are selected from the group consisting of beads, polyhedrons, hydrogel pieces, and microsponges.

8. The device in claim 6 wherein flexible longitudinal members are selected from the group consisting of thin wires, strands, strings, springs, sutures, ribbons, and filaments.

9. An intrasaccular aneurysm occlusion device comprising:
a distal mesh which is inserted into an aneurysm sac, wherein the distal mesh is cardioid shaped, wherein the distal mesh centroid is a first distance from the aneurysm neck; and
a bowl-shaped proximal mesh inserted into the aneurysm sac, wherein the proximal mesh centroid is a second distance from the aneurysm neck;
wherein the second distance is less than the first distance, wherein the proximal mesh spans the aneurysm neck, wherein a portion of the distal mesh is within the concavity of the proximal mesh, wherein the proximal mesh is axially-aligned with the distal mesh, and wherein the distal mesh is filled with embolic material.

10. The device in claim 9 wherein the distal mesh is filled with embolic coils.

11. The device in claim 9 wherein the distal mesh is filled with string-of-pearls embolic strands.

12. The device in claim 11 wherein a string-of-pearls embolic strand is a series or sequence of embolic members which are connected by flexible longitudinal members.

13. The device in claim 12 wherein embolic members are selected from the group consisting of beads, polyhedrons, hydrogel pieces, and microsponges.

14. The device in claim 12 wherein flexible longitudinal members are selected from the group consisting of thin wires, strands, strings, springs, sutures, ribbons, and filaments.

15. An intrasaccular aneurysm occlusion device comprising:
a limacon-shaped distal portion which is inserted into an aneurysm sac; and
a concave proximal portion which is inserted into the aneurysm sac and expands to cover the aneurysm neck;
wherein the limacon-shaped distal portion is between the concave proximal portion and the dome of the aneurysm sac, wherein at least part of the limacon-shaped distal portion is nested within a concavity of the concave proximal portion, and wherein the limacon-shaped distal portion is connected to the concave proximal portion at a proximal location on the limacon-shaped distal portion.

16. The device in claim 15 wherein embolic coils are inserted into the distal portion.

17. The device in claim 15 wherein string-of-pearls embolic strands are inserted into the distal portion.

18. The device in claim 17 wherein a string-of-pearls embolic strand is a series or sequence of embolic members which are connected by flexible longitudinal members.

19. The device in claim 18 wherein embolic members are selected from the group consisting of beads, polyhedrons, hydrogel pieces, and microsponges.

20. The device in claim 18 wherein flexible longitudinal members are selected from the group consisting of thin wires, strands, strings, springs, sutures, ribbons, and filaments.
